(19) United States
(12) Patent Application Publication
(10) Pub. No.: US 2025/0092396 A1
(43) Pub. Date: Mar. 20, 2025

(54) Treatment Of Osteoarthritis With Cartilage Intermediate Layer Protein 2 (CILP2) Inhibitors

(71) Applicant: Regeneron Pharmaceuticals, Inc., Tarrytown, NY (US)

(72) Inventors: Jonas Bovijn, Tarrytown, NY (US); Jose Raya Garcia del Olmo, Tarrytown, NY (US); Peter Dornbos, Tarrytown, NY (US); Benjamin Geraghty, Tarrytown, NY (US); Jonathan Marchini, Tarrytown, NY (US); Aris Baras, Tarrytown, NY (US); Mary Haas, Tarrytown, NY (US); Luca Andrea Lotta, Tarrytown, NY (US)

(21) Appl. No.: 18/887,308

(22) Filed: Sep. 17, 2024

Related U.S. Application Data
(60) Provisional application No. 63/538,996, filed on Sep. 18, 2023.

(51) Int. Cl. C12N 15/113; A61P 19/02

(52) U.S. Cl. CPC C12N 15/113; A61P 19/02; C12N 2310/11; C12N 2310/14; C12N 2310/531

(57) ABSTRACT

The present disclosure generally relates to the treatment of subjects having osteoarthritis or at risk of developing osteoarthritis by administering a Cartilage Intermediate Layer Protein 2 (CILP2) inhibitor to the subject.

### FIELD

The present disclosure generally relates to the treatment of subjects having osteoarthritis or at risk of developing osteoarthritis, by administering a Cartilage Intermediate Layer Protein 2 (CILP2) inhibitor to the subject, and to methods of identifying subjects having an increased risk of developing osteoarthritis.

### BACKGROUND

Osteoarthritis (OA) is the most common form of arthritis and affects millions worldwide. OA is a degenerative joint disease that affects all tissues in the joint. Damage to the articular cartilage, a soft tissue layer covering the bony ends, occurs early in the disease and is considered the onset of irreversible joint damage. Although OA can involve any joint, the most commonly affected joints are the knees, hip, hands, and spine. OA can be diagnosed by a physical examination and may include imaging tests (X-rays) to assess severity, and laboratory tests (such as, for example, blood or urine tests and joint fluid analysis). Symptoms of OA include: i) pain in joints, ii) joint stiffness upon awakening or after being inactive, iii) tenderness in joint when light pressure is applied to or near it, iv) loss of flexibility, v) grating sensation when using the joint (popping or crackling), vi) bone spurs can form around the affected joint, and vii) swelling (soft tissue inflammation around the joint). Risk factors for OA include: i) older age (e.g., risk of OA increases with age), ii) obesity (increased weight adds stress to weight-bearing joints and fat tissue produces proteins that can cause harmful inflammation in and around joints), iii) joint injuries, iv) sex (females are more likely to develop OA), v) repeated stress on the joint, vi) genetics, vii) bone deformities, and viii) some metabolic diseases (such as diabetes and hemochromatosis).

Cartilage Intermediate Layer Protein 2 (CILP2) is expressed by articular chondrocytes. CILP2 is highly homologous to cartilage intermediate layer protein 1 (CILP1), which is expressed in the intermediate zone of articular cartilage and has been linked to cartilage degenerative diseases. CILP2 is expressed at the surface of the mouse articular cartilage during development. With maturity CILP2 expression becomes localized around the central main layer of articular cartilage and meniscus. In humans, CILP2 was shown to be proteolytically processed, N-glycosylated, and present in articular cartilage. In a mouse model of surgically-induced OA, CILP1 expression was up-regulated but CILP2 gene expression was down-regulated demonstrating a differential response to joint destabilization leading to a reduction in CILP2 protein. Ultrastructural analysis also suggested that CILP2 may be associated with collagen VI microfibrils and may mediate interactions between matrix components in the territorial and inter-territorial extracellular matrix. mRNA expression analysis indicated that whereas CILP1 and CILP2 are expressed most abundantly in cartilaginous tissues, expression can be detected in muscle and heart. (Bernardo et al., J. Biol. Chem., 2011, 286, 37758-37767).

### SUMMARY

The present disclosure provides methods of treating a subject having OA, or at risk of developing OA, the methods comprising administering a CILP2 inhibitor to the subject.

The present disclosure also provides methods of treating a subject having OA or at risk of developing OA by administering an OA therapeutic agent or OA therapy, the methods comprising: determining or having determined whether the subject has a CILP2 variant nucleic acid molecule, by: obtaining or having obtained a biological sample from the subject; and performing or having performed a sequence analysis on the biological sample to determine if the subject has a genotype comprising a CILP2 variant nucleic acid molecule; and administering or continuing to administer the OA therapeutic agent in an amount that is the same as or less than a standard dosage amount or OA therapy, and/or administering a CILP2 inhibitor to a subject that is CILP2 reference; administering or continuing to administer the OA therapeutic agent in an amount that is the same as or less than a standard dosage amount or OA therapy, and/or administering a CILP2 inhibitor to a subject that is heterozygous for the CILP2 variant nucleic acid molecule; or administering or continuing to administer the OA therapeutic agent or OA therapy in a standard dosage amount to a subject that is homozygous for the CILP2 variant nucleic acid molecule; wherein the presence the CILP2 variant nucleic acid molecule indicates the subject has a decreased risk of developing OA.

The present disclosure also provides methods of identifying a subject having an increased risk of developing OA, the methods comprising: determining or having determined the presence or absence of a CILP2 variant nucleic acid molecule in a biological sample obtained from the subject; wherein: when the subject is CILP2 reference, then the subject has an increased risk of developing OA; and when the subject is heterozygous or homozygous for the CILP2 variant nucleic acid molecule, then the subject has a decreased risk of developing OA.

The present disclosure also provides OA therapeutic agents for use in the treatment or prevention of OA in a subject having a CILP2 variant nucleic acid molecule.

The present disclosure also provides CILP2 inhibitors for use in the treatment or prevention of OA in a subject that is CILP2 reference or is heterozygous for a CILP2 variant nucleic acid molecule.

### BRIEF DESCRIPTION OF THE DRAWINGS

FIG. 1 shows representative associations between CILP2 pLoF and missense variants and minimum joint space width (mJSW) phenotype derived from D×A images (Panel A) and between CILP2 pLoF variants and knee OA (Panel B).

FIG. 2 shows associations between CILP2 pLoF variants and OA in various joints.

FIG. 3 shows association between pLoF variants in CILP2 and lower risk of knee replacement.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that rare CILP2 variant nucleic acid molecules (whether these variants are homozygous or heterozygous in a particular subject) associated with a decreased risk of developing OA. In particular, a knee minimum Joint Space Width (mJSW) phenotype was derived from UK Biobank DXA imaging (N=51,327) and used for target discovery and validation. mJSW showed expected correlations with sex, height, age and body mass index (BMI), and between left and right knees and baseline and follow up scans (n=4,000). mJSW was reduced in OA cases in men (p=2.1e−4, N=24,567) and women (p=1.9e−4, N=25,673). CILP2 pLoF burden (AAF<1%) was associated with 0.78 standrad deviation (SD) units greater mJSW (0.5 mm; p-value=5.7e−9) and was observed to be protective for OA (odds ratio=0.56; p-value=8.3e−4). It is believed that CILP2 variant nucleic acid molecules have not been associated with OA in humans. Therefore, subjects that are CILP2 reference or heterozygous for a CILP2 variant nucleic acid molecule may be treated with a CILP2 inhibitor such that OA is inhibited or prevented, the symptoms thereof are reduced or prevented, and/or development of symptoms is repressed or prevented. It is also believed that such subjects having OA may further be treated with one or more OA therapeutic agents or OA therapy that treats or inhibits OA. In addition, the present disclosure provides methods of leveraging the presence or absence of CILP2 variant nucleic acid molecules in subjects to identify or stratify risk is such subjects of developing OA, or to diagnose subjects as having an increased risk of developing OA.

For purposes of the present disclosure, any particular subject, such as a human, can be categorized as having one of three CILP2 genotypes: i) CILP2 reference; ii) heterozygous for a CILP2 variant nucleic acid molecule; or iii) homozygous for a CILP2 variant nucleic acid molecule. A subject is CILP2 reference when the subject does not have a copy of a CILP2 variant nucleic acid molecule. A subject is heterozygous for a CILP2 variant nucleic acid molecule when the subject has a single copy of a CILP2 variant nucleic acid molecule. A subject is homozygous for a CILP2 variant nucleic acid molecule when the subject has two copies of a CILP2 variant nucleic acid molecule.

In any of the embodiments described herein, the CILP2 variant nucleic acid molecule can be any nucleic acid molecule (such as, a genomic nucleic acid molecule, an mRNA molecule, or a cDNA molecule produced from an mRNA molecule) encoding a CILP2 variant polypeptide having a partial loss-of-function, a complete loss-of-function, a predicted partial loss-of-function, or a predicted complete loss-of-function. A subject who has a CILP2 polypeptide having a partial loss-of-function (or predicted partial loss-of-function) is hypomorphic for CILP2. In some embodiments, the CILP2 variant nucleic acid molecule results in decreased or aberrant expression or activity of CILP2 mRNA or polypeptide. In some embodiments, the CILP2 variant nucleic acid molecule is associated with a reduced in vitro response to CILP2 ligands compared with reference CILP2. In some embodiments, the CILP2 variant nucleic acid molecule is a splice-site variant, a stop-gain variant, a start-loss variant, a stop-loss variant, a frameshift variant, an in-frame indel variant, or a variant that encodes a truncated CILP2 variant polypeptide. In some embodiments, the CILP2 variant nucleic acid molecule is a missense variant nucleic acid molecule. In some embodiments, the CILP2 variant nucleic acid molecule comprises a single nucleotide polymorphism (SNP). In some embodiments, the CILP2 variant nucleic acid molecule comprises a variation in a coding region. In some embodiments, the CILP2 variant nucleic acid molecule does not comprise a variation in a non-coding region, except for a splice acceptor region (two bases before the start of any exon except the first). In some embodiments, the CILP2 variant nucleic acid molecule results or is predicted to result in a premature truncation of a CILP2 polypeptide compared to the reference CILP2. In some embodiments, the CILP2 variant nucleic acid molecule is a variant that is predicted to be damaging to the protein function (and hence, in this case, protective to the human) by in vitro prediction algorithms such as Polyphen, SIFT, or similar algorithms. In some embodiments, the CILP2 variant nucleic acid molecule is a variant that causes or is predicted to cause a nonsynonymous amino acid substitution in a CILP2 nucleic acid molecule and whose allele frequency is less than 1/100 alleles in the population from which the subject is selected. In some embodiments, the CILP2 variant nucleic acid molecule is any rare missense variant (allele frequency <0.1%; or 1 in 1,000 alleles), or any splice-site, stop-gain, start-loss, stop-loss, frameshift, or in-frame indel, or other frameshift CILP2 variant.

In any of the embodiments described herein, the CILP2 variant genomic nucleic acid molecule may include one or more variations at any of the positions of chromosome 19 (i.e., positions 19,538,248-19,546,659) using the nucleotide sequence of the CILP2 reference genomic nucleic acid molecule in the GRCh38/hg38 human genome assembly (see, ENSG00000160161.9, ENSTENST00000291495 annotated in the in the Ensembl database (URL: world wide web at “http://useast.ensembl.org/Homo_sapiens/Gene/Summary? g=ENSG00000160161; r=19:19538248-19546659)) as a reference sequence. The sequences provided in these transcripts for the CILP2 genomic nucleic acid molecule are only exemplary sequences. Other sequences for the CILP2 genomic nucleic acid molecule are also possible.

In any of the embodiments described herein, the CILP2 variant nucleic acid molecule may comprise any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

For subjects that are genotyped or determined to be CILP2 reference, such subjects have an increased risk of developing OA. For subjects that are genotyped or determined to be either CILP2 reference or heterozygous for a CILP2 variant nucleic acid molecule, such subjects can be treated with a CILP2 inhibitor.

In any of the embodiments described herein, the subject in whom OA is prevented by administering a CILP2 inhibitor may be anyone at risk for developing OA including, but not limited to, subjects with a genetic predisposition for developing OA. In some embodiments, administering a CILP2 inhibitor to a subject having OA may be carried out to prevent development of another occurrence of OA in a subject who has already had OA. In any of the embodiments described herein, the methods can be used to improve OA.

In any of the embodiments described herein, the CILP2 predicted loss-of-function polypeptide can be any CILP2 polypeptide having a partial loss-of-function, a complete loss-of-function, a predicted partial loss-of-function, or a predicted complete loss-of-function.

Any one or more (i.e., any combination) of the CILP2 variant nucleic acid molecules described herein can be used within any of the methods described herein to determine whether a subject has an increased or decreased risk of developing OA. The combinations of particular variants can form a mask used for statistical analysis of the particular correlation of CILP2 and an increased or decreased risk of developing OA. In some embodiments, the mask used for statistical analysis of the particular correlation of CILP2 and an increased or decreased risk of developing OA can exclude any one or more of these CILP2 variant nucleic acid molecules described herein.

In any of the embodiments described herein, the subject can have OA. In any of the embodiments described herein, the subject can be at risk of developing OA.

The present disclosure provides methods of treating a subject having OA or at risk of developing OA, the methods comprising administering a CILP2 inhibitor to the subject.

The present disclosure also provides methods of preventing the need or prolonging the need for a joint replacement, such as a knee replacement and/or hip replacement, in a subject, the methods comprising administering a CILP2 inhibitor to the subject. Such prolonging can be, for example, months to years. For example, a subject that has an increased risk of requiring a joint replacement (e.g., knee and/or hip) can be administered a CILP2 inhibitor in order to prolong the time period before an actual joint replacement surgery, or prevent the actual joint replacement altogether. In some embodiments, the need for a joint replacement surgery is an outcome of osteoarthritis.

In some embodiments, the CILP2 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CILP2 nucleic acid molecule. In some embodiments, the antisense RNA, siRNA, or shRNA hybridizes to a sequence within a CILP2 genomic nucleic acid molecule or mRNA molecule and decreases expression of the CILP2 polypeptide in a cell in the subject. In some embodiments, the CILP2 inhibitor comprises an antisense molecule that hybridizes to a CILP2 genomic nucleic acid molecule or mRNA molecule and decreases expression of the CILP2 polypeptide in a cell in the subject. In some embodiments, the CILP2 inhibitor comprises an siRNA that hybridizes to a CILP2 genomic nucleic acid molecule or mRNA molecule and decreases expression of the CILP2 polypeptide in a cell in the subject. In some embodiments, the CILP2 inhibitor comprises an shRNA that hybridizes to a CILP2 genomic nucleic acid molecule or mRNA molecule and decreases expression of the CILP2 polypeptide in a cell in the subject. An exemplary siRNA is sc-60386 (Santa Cruz Biotechnology, Inc.).

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:
Sense: mN*mN*/i2FN/mN/i2FN/mN/i2FN/mN/i2FN/mN/i2FN/mN/i2FN/mN/i2FN/mN/12FN/*mN*/32FN/
Antisense:/52FN/*/i2FN/*mN/i2FN/mN/i2FN/mN/i2FN/mN/i2FN/mN/i2FN/mN/i2FN/mN/12FN/mN/i2FN/mN*N*N
wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²-based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly (lactic acid) (PLA) microspheres, poly (D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the CILP2 inhibitor comprises a nuclease agent that induces one or more nicks or double-strand breaks at a recognition sequence(s) or a DNA-binding protein that binds to a recognition sequence within a CILP2 genomic nucleic acid molecule. The recognition sequence can be located within a coding region of the CILP2 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the CILP2 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a CILP2 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of CILP2 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a CILP2 genomic nucleic acid molecule or it can be a nickase that creates a single-strand break in a CILP2 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of CILP2 genomic nucleic acid molecules can be generated by contacting a cell with a Cas protein and one or more gRNAs that hybridize to one or more gRNA recognition sequences within a target genomic locus in the CILP2 genomic nucleic acid molecule. The gRNA recognition sequence can include or be proximate to the start codon of a CILP2 genomic nucleic acid molecule or the stop codon of a CILP2 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a CILP2 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where Nis any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a CILP2 genomic nucleic acid molecule. An exemplary gRNA is a gRNA effective to direct a Cas enzyme to bind to or cleave a CILP2 genomic nucleic acid molecule, wherein the gRNA comprises a DNA-targeting segment that hybridizes to a gRNA recognition sequence within the CILP2 genomic nucleic acid molecule. Exemplary gRNAs comprise a DNA-targeting segment that hybridizes to a gRNA recognition sequence present within a CILP2 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the CILP2 genomic nucleic acid molecule. The Cas protein can cleave the nucleic acid molecule at a site within or outside of the nucleic acid sequence present in the CILP2 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the CILP2 genomic nucleic acid molecule to which a DNA-targeting segment of a gRNA will bind.

Such methods can result, for example, in a CILP2 genomic nucleic acid molecule in which a region of the CILP2 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the CILP2 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In any of the methods of treatment or prevention described herein, the subject being treated may comprise a CILP2 variant nucleic acid molecule. In some embodiments, the subject being treated is heterozygous for the CILP2 variant nucleic acid molecule. In some embodiments, the subject being treated is homozygous for the CILP2 variant nucleic acid molecule. In some embodiments, the subject being treated is CILP2 reference. The CILP2 variant nucleic acid molecule can be any of the CILP2 variant nucleic acid molecules disclosed herein. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

In some embodiments, the methods of treatment or prevention further comprise detecting the presence or absence of a CILP2 variant nucleic acid molecule in a biological sample from the subject. In some embodiments, the CILP2 variant nucleic acid molecule can be any of the CILP2 variant nucleic acid molecules disclosed herein. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

The present disclosure also provides methods of treating a subject with an OA therapeutic agent or OA therapy that treats or inhibits OA, wherein the subject has OA or is at risk of developing OA. The methods comprise determining whether the subject has a CILP2 variant nucleic acid molecule by obtaining or having obtained a biological sample from the subject, and performing or having performed a sequence analysis on the biological sample to determine if the subject has a genotype comprising the CILP2 variant nucleic acid molecule. In embodiments where the subject is CILP2 reference, the methods further comprise administering or continuing to administer the OA therapeutic agent in an amount that is the same as or less than a standard dosage amount or OA therapy to the subject, and/or administering a CILP2 inhibitor to the subject. In embodiments where the subject is heterozygous for the CILP2 variant nucleic acid molecule, the methods further comprise administering or continuing to administer the OA therapeutic agent in an amount that is the same as or less than a standard dosage amount or OA therapy to the subject, and/or administering a CILP2 inhibitor to the subject. In embodiments where the subject is homozygous for the CILP2 variant nucleic acid molecule, the methods further comprise administering or continuing to administer the OA therapeutic agent in a standard dosage amount or OA therapy to the subject. The presence of a CILP2 variant nucleic acid molecule indicates the subject has a decreased risk of developing OA. In some embodiments, the subject is CILP2 reference. In some embodiments, the subject is heterozygous for a CILP2 variant nucleic acid molecule. In some embodiments, the subject is homozygous for a CILP2 variant nucleic acid molecule. In any of the embodiments described herein, the CILP2 inhibitor is an example of an OA therapeutic agent. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

For subjects that are genotyped or determined to be either CILP2 reference or heterozygous for a CILP2 variant nucleic acid molecule, such subjects can be administered a CILP2 inhibitor, as described herein.

Detecting the presence or absence of a CILP2 variant nucleic acid molecule in a biological sample from a subject and/or determining whether a subject has a CILP2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is CILP2 reference, the subject is administered an OA therapeutic agent in an amount that is the same as or less than a standard dosage amount or OA therapy, and/or a CILP2 inhibitor. In some embodiments, when the subject is heterozygous for a CILP2 variant nucleic acid molecule, the subject is administered an OA therapeutic agent in an amount that is the same as or less than a standard dosage amount or OA therapy, and/or a CILP2 inhibitor.

In some embodiments, the treatment or prevention methods comprise detecting the presence or absence of a decrease in the expression of a CILP2 variant mRNA or polypeptide in a biological sample from the subject. In some embodiments, when the subject does not have a decrease in the expression of a CILP2 variant mRNA or polypeptide, the subject is administered an OA therapeutic agent in an amount that is the same as or less than a standard dosage amount or OA therapy, and/or a CILP2 inhibitor. In some embodiments, when the subject has a decrease in the expression of a CILP2 variant mRNA or polypeptide, the subject is administered an OA therapeutic agent in a standard dosage amount or OA therapy.

The present disclosure also provides methods of treating a subject with an OA therapeutic agent or OA therapy that treats or inhibits OA, wherein the subject has OA or is at risk of developing OA. The methods comprise determining whether the subject has a decrease in the expression of a CILP2 variant mRNA or polypeptide by obtaining or having obtained a biological sample from the subject, and performing or having performed an assay on the biological sample to determine if the subject a decrease in the expression of a CILP2 variant mRNA or polypeptide. In embodiments where the subject does not have a decrease in the expression of a CILP2 variant mRNA or polypeptide, the methods further comprise administering or continuing to administer the OA therapeutic agent in an amount that is the same as or less than a standard dosage amount or OA therapy to the subject, and/or administering a CILP2 inhibitor to the subject. In embodiments where the subject has a decrease in the expression of a CILP2 variant mRNA or polypeptide, the methods further comprise administering or continuing to administer the OA therapeutic agent in a standard dosage amount or OA therapy to the subject. The presence of a decrease in the expression of a CILP2 variant mRNA or polypeptide indicates the subject has a decreased risk of developing OA. In some embodiments, the subject has a decrease in the expression of a CILP2 variant mRNA or polypeptide. In some embodiments, the subject does not have a decrease in the expression of a CILP2 variant mRNA or polypeptide. In any of the embodiments described herein, the CILP2 inhibitor is an example of an OA therapeutic agent. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

Detecting a decrease in the expression of a CILP2 variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment or prevention methods comprise detecting the presence or absence of a CILP2 variant polypeptide in a biological sample from the subject. In some embodiments, when the subject does not have a CILP2 variant polypeptide, the subject is administered an OA therapeutic agent in an amount that is the same as or less than a standard dosage amount or OA therapy, and/or a CILP2 inhibitor. In some embodiments, when the subject has a CILP2 variant polypeptide, the subject is administered an OA therapeutic agent in standard dosage amount or OA therapy.

The present disclosure also provides methods of treating a subject with an OA therapeutic agent or OA therapy that treats or inhibits OA, wherein the subject has OA or is at risk of developing OA. The methods comprise determining whether the subject has a CILP2 variant polypeptide by obtaining or having obtained a biological sample from the subject and performing or having performed an assay on the biological sample to determine if the subject has a CILP2 variant polypeptide. When the subject does not have a CILP2 variant polypeptide, the subject is administered the OA therapeutic agent in an amount that is the same as or less than a standard dosage amount or OA therapy, and/or a CILP2 inhibitor. When the subject has a CILP2 variant polypeptide, the subject is administered the OA therapeutic agent in a standard dosage amount or OA therapy. The presence of a CILP2 variant polypeptide indicates the subject has a decreased risk of developing OA. In some embodiments, the subject has a CILP2 variant polypeptide. In some embodiments, the subject does not have a CILP2 variant polypeptide.

The present disclosure also provides methods of preventing a subject from developing OA by administering an OA therapeutic agent or OA therapy that prevents OA. In some embodiments, the method comprises determining whether the subject has a CILP2 variant polypeptide by obtaining or having obtained a biological sample from the subject and performing or having performed an assay on the biological sample to determine if the subject has a CILP2 variant polypeptide. When the subject does not have a CILP2 variant polypeptide, the subject is administered the OA therapeutic agent in an amount that is the same as or less than a standard dosage amount or OA therapy, and/or a CILP2 inhibitor. When the subject has a CILP2 variant polypeptide, the subject is administered the OA therapeutic agent in a standard dosage amount or OA therapy. The presence of a CILP2 variant polypeptide indicates the subject has a decreased risk of developing OA. In some embodiments, the subject has a CILP2 variant polypeptide. In some embodiments, the subject does not have a CILP2 variant polypeptide.

Detecting the presence or absence of a CILP2 variant polypeptide in a biological sample from a subject and/or determining whether a subject has a CILP2 variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the CILP2 inhibitor is a small molecule. In some embodiments, the small molecule is low molecular weight (<900 daltons) organic compound.

In some embodiments, the CILP2 inhibitor comprises an antibody, or antigen-binding fragment thereof. In some embodiments, the antibody, or antigen-binding fragment thereof, binds specifically to human CILP2. In some embodiments, the antibody is a fully human monoclonal antibody (mAb), or antigen-binding fragment thereof, that specifically binds and neutralizes, inhibits, blocks, abrogates, reduces, or interferes with, at least one activity of CILP2, in particular human CILP2. In some embodiments, an antibody or fragment thereof can neutralize, inhibit, block, abrogate, reduce, or interfere with, an activity of CILP2 by binding to an epitope of CILP2 that is directly involved in the targeted activity of CILP2. In some embodiments, an antibody or fragment thereof can neutralize, inhibit, block, abrogate, reduce, or interfere with, an activity of CILP2 by binding to an epitope of CILP2 that is not directly involved in the targeted activity of CILP2, but the antibody or fragment binding thereto sterically or conformationally inhibits, blocks, abrogates, reduces, or interferes with, the targeted activity of CILP2. In some embodiments, an antibody or fragment thereof binds to an epitope of CILP2 that is not directly involved in the targeted activity of CILP2 (i.e., a non-blocking antibody), but the antibody or fragment binding thereto results in the enhancement of the clearance of CILP2 from the circulation, compared to the clearance of CILP2 in the absence of the antibody or fragment thereof, thereby indirectly inhibiting, blocking, abrogating, reducing, or interfering with, an activity of CILP2. Clearance of CILP2 from the circulation can be particularly enhanced by combining two or more different non-blocking antibodies that do not compete with one another for specific binding to CILP2. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the antibody or antigen-binding fragment thereof specifically binds to CILP2 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-CILP2 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made to modify complement dependent cytotoxicity (CDC).

The present disclosure also provides compositions comprising a combination of an antibody or antigen-binding fragment thereof and an OA therapeutic agent.

In some embodiments, the OA therapeutic agents include, but are not limited to, medications (such as, for example, acetaminophen, a nonsteroidal anti-inflammatory drug (NSAID) (such as, for example, ibuprofen and naproxen sodium), duloxetine, a corticosteroid (such as, for example, cortisone)), a lubricator (such as, for example, hyaluronic acid), and an analgesic, such as an opioid, or any combination thereof. In some embodiments, the OA therapeutic agent comprises acetaminophen. In some embodiments, the OA therapeutic agent comprises a nonsteroidal anti-inflammatory drug (NSAID). In some embodiments, the NSAID comprises ibuprofen. In some embodiments, the NSAID comprises naproxen sodium. In some embodiments, the OA therapeutic agent comprises duloxetine. In some embodiments, the OA therapeutic agent comprises a corticosteroid. In some embodiments, the corticosteroid comprises cortisone. In some embodiments, the OA therapeutic agent comprises a lubricator. In some embodiments, the lubricator comprises hyaluronic acid. In some embodiments, the OA therapeutic agent comprises an analgesic. In some embodiments, the alagesic comprises an opioid. In some embodiments, the OA therapeutic agent comprises a Nerve Growth Factor (NGF) inhibitor. In some embodiments, the OA therapeutic agent comprises fasinumab.

In some embodiments, the OA therapy comprises any therapy used to reduce or manage OA. In some embodiments, the OA therapy comprises physical therapy, occupational therapy, transcutaneous electrical nerve stimulation (TENS), or surgical and other procedures (such as, for example, knee osteotomy, joint replacement (e.g., knee and/or hip replacement), and bone realignment), or any combination thereof. These treatment therapies may be delayed or avoided altogether by treatment with a CILP2 inhibitor as described herein.

In some embodiments, the OA therapy comprises a cartilage repair technique such as, for example, an autologous chondrocyte transplant. In some embodiments, chondrocytes can be obtained from a particular subject to be treated. These obtained chondrocytes cultured with a CILP2 inhibitor and reimplanted into the subject. In some embodiments, the cultured chondrocytes can be reimplanted embedded on a matrix on a cartilage defect in the subject. In some embodiments, the reimplanted chondrocytes are chondrocyte stem cells. In some embodiments, the reimplanted chondrocytes have been genetically modified, such as by CRISPR techniques, to contain a CILP2 gene that comprises any one or more of the genetic variations in the genomic nucleic acid molecule (referring to the chromosome: positions set forth in the GRCh38/hg38 human genome assembly) set forth in Table 1 (heterozygous or homozygous). In such cases, the reimplanted chondrocytes need not be cultured with a CILP2 inhibitor prior to reimplantation into the subject.

In some embodiments, the OA therapeutic agent or OA therapy can be combined with a CILP2 inhibitor.

In some embodiments, the dose of the OA therapeutic agents that treat, prevent, or inhibit OA can be decreased by about 10%, by about 20%, by about 30%, by about 40%, by about 50%, by about 60%, by about 70%, by about 80%, or by about 90% for subjects that are heterozygous for a CILP2 variant nucleic acid molecule or CILP2 reference (i.e., a less than the standard dosage amount) compared to subjects that are homozygous for a CILP2 variant nucleic acid molecule (who may receive a standard dosage amount). In some embodiments, the dose of the OA therapeutic agents that treat, prevent, or inhibit OA can be decreased by about 10%, by about 20%, by about 30%, by about 40%, or by about 50%. In some embodiments, the dose of the OA therapeutic agents that treat, prevent, or inhibit OA can be decreased by about 10%, by about 20%, by about 30%, by about 40%, by about 50%, by about 60%, by about 70%, by about 80%, or by about 90% for subjects that are heterozygous for a CILP2 variant nucleic acid molecule or CILP2 reference compared to subjects that are CILP2 reference. In addition, subjects that are heterozygous for a CILP2 variant nucleic acid molecule or CILP2 reference can be administered the OA therapeutic agents less frequently compared to subjects that are heterozygous for the CILP2 variant nucleic acid molecule.

Administration of the OA therapeutic agents that treat, prevent, or inhibit OA and/or CILP2 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the OA therapeutic agents and/or CILP2 inhibitors can occur by any suitable route including, but not limited to, intraarticular, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in OA, a decrease/reduction in the severity of OA (such as, for example, a reduction or inhibition of development of OA), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to OA, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of OA development/progression (such as, for example, a complete or partial avoidance/inhibition or a delay), and an increased survival time of the affected host animal, following administration of a therapeutic protocol. Treatment of OA encompasses the treatment of a subject already diagnosed as having any form of OA at any clinical stage or manifestation, the delay of the onset or evolution or aggravation or deterioration of the symptoms or signs of OA, and/or preventing and/or reducing the severity of OA.

In some embodiments, the CILP2 inhibitor and the OA therapeutic agent are disposed within a pharmaceutical composition. In some embodiments, the CILP2 inhibitor is disposed within a first pharmaceutical composition and the OA therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

In any of the embodiments described herein, the presence and severity of OA can be assessed by imaging. In some embodiments, the present state of OA in a subject can be assessed by imaging. In some embodiments, the progression of OA in a subject can be assessed by imaging. In some embodiments, the lack of progression of OA in a subject can be assessed by imaging. In some embodiments, the efficacy of a therapeutic treatment of OA can be monitored by imaging.

In any of the embodiments described herein, the imaging can be any medical imaging. The medical imaging modality includes, but is not limited to, weight-bearing X-ray, dual X-ray absorptiometry (DXA), magnetic resonance imaging (MRI), computerized tomography (CT), or positron emission tomography (PET). The image sites include, but are not limited to, knees, hips, hands, spine, and shoulder.

In some embodiments, images are obtained by measuring the absorption of X-ray radiation (weight-bearing X-ray, DXA, or CT), capturing localized measures of bone mineral density. In such embodiments, a 2-dimensional (2D) projection or 3D reconstruction allows quantification of bone structure and integrity that can be used as a biomarker for the assessment of OA. Several methods can be used to derive biomarkers from the images, including, but not limited to, segmentation of articulating bones to measure the JSW (e.g., the femur and the tibia), direct measurement of JSW on the images using an electronic caliper, or assessment of the severity of disease by an experienced reader using validated clinical scores, or quantification of the trabecular, subchondral and cortical bone regions.

In some embodiments, images are obtained based on the principle of resonant excitation of the tissue magnetization induced by an external magnetic field. The image is obtained by encoding the spatial position into the precession frequency of the magnetization using magnetic field gradients. Images obtained in such a manner provide excellent soft tissue contrast and allow assessment of all connective tissue components in the joint. Biomarkers can be extracted from the images by different methods, including but not limited to changing the contrast of the image according to a specified scheme to derive quantitative measures (e.g., measurement of T2 relaxation time measured by acquisitions with different echo times), or using a contrast agent to enhance or quantify a process (e.g., contrast-enhanced MRI with Gd contrast agent for inflammation), or by quantifying the anatomy of the joint from the images (e.g., measuring the cartilage thickness).

In some embodiments, a radioactive contrast agent is used to track where this agent accumulates in the body. This contrast agent has a component with molecular specificity for binding/uptake and a radioactive element for which the by-product produces a particle that can be detected directly or indirectly (e.g., PET). Images acquired using this principle provide many biomarkers, including but not limited to, the standardized uptake value, the tissue volume, or parameters from a pharmacokinetic model fitted to the measured signal.

In any of the methods described herein, the measurements derived from the images can be used as a marker of the presence of OA, severity of OA, prediction of progression (or lack thereof) of OA, or assessment of therapy efficacy. For example, a subject who is suspected of having or developing OA may obtain from the affected joint an initial image (such as an X-ray) and a follow-up image or series of images with the same modality. At each timepoint, the same imaging marker will be calculated using the same methods to assess changes. X-ray imaging of affected joints can be performed with additional imaging as desired. In some embodiments, sensitive definitions and prospective imaging are used. In some embodiments, MRI is frequently used at baseline and for adjudication which is superior to conventional radiography at detecting joint pathology. For example, a reduction in the JSW in the affected joint over time will indicate that the OA is progressing in that joint. A subject may desire to be treated, such as by any of the methods described herein. Any of the herein-described imaging methods can monitor the efficacy of treatment to determine the efficacy of the treatment.

In some embodiments, patients treated with a specific therapeutic agent show an average change in an imaging marker (e.g., an increase in JSW) that is significantly different from the average change in patients treated with placebo, which can be interpreted as a positive measure of improvement. Such change would be interpreted as a positive efficacy of the therapy.

In some embodiments, the subject can have rapidly progressive osteoarthritis type 1 (RPOA-1) (e.g., joint space narrowing), rapidly progressive osteoarthritis type 2 (RPOA-2) (e.g., limited/partial joint collapse; bone damage), primary osteonecrosis (e.g., avascular necrosis), subchondral insufficiency fracture (SIF), or destructive arthropathy (DA) (e.g., complete joint collapse). In some embodiments, the subject has RPOA-1. In some embodiments, the subject has RPOA-2. In some embodiments, the subject has primary osteonecrosis. In some embodiments, the subject has SIF. In some embodiments, the subject has DA.

In some embodiments, RPOA-1 can be characterized as a rapid loss of joint space width from baseline without evidence of bone fragmentation or destruction. If rapid loss of JSW from baseline is observed by X-ray, an MRI can be obtained and substantial focal or diffuse loss of hyaline/articular cartilage from baseline consistent with RPOA 1 can be observed. A rapid change in joint space width from baseline is defined as: a) knee joints: if JSW is ≥2 mm at baseline, a decrease of ≥2 mm or 50% from baseline JSW at any point during the study (whichever is greater); and if JSW is <2 mm at baseline or where accurate JSW measurement is not possible, a change in JSW to 0 mm; b) hip joints: if JSW is ≥1.5 mm at baseline, a decrease of >1.5 mm from baseline; and if JSW is <1.5 mm at baseline or where accurate JSW measurement is not possible, a change in JSW to 0 mm. If a prior image of the same joint is not available for comparison, by definition, RPOA Type 1 cannot be determined.

In some embodiments, RPOA-2 can be characterized as abnormal bone fragmentation or destruction over a short period of time, including limited collapse of at least one articular surface, and are observed principally by MRI but may be detected by X-rays.

In some embodiments, primary osteonecrosis can be characterized as focal circumscribed or extended region of mottled radiolucency and sclerosis (infarcted bone) which is confirmed by MRI. No evidence of subchondral collapse or bone fragmentation preceding or concurrent with the diagnosis of primary osteonecrosis.

In some embodiments, SIF can be characterized as subchondral radiolucency, which may have a sclerotic linear component and articular surface flattening and is confirmed by MRI. Does not include significant collapse or fragmentation.

In some embodiments, DA can be characterized as abnormal bone fragmentation, destruction or fracture over a short period of time, including near-total collapse of an articular surface, and often associated with subluxation or malalignment, all of which are features inconsistent with radiographic findings typically observed in conventional advanced OA, and are readily observed by x-rays.

In some embodiments, when a subject has DA, particular parameters of treatment can be tailored. For example, a) treatment can be restricted to patient populations that are refractory to or unable to tolerate acetaminophen, NSAIDs, and opioids; b) patients with co-morbidities that may result in an increased risk for destructive arthropathy can be excluded; c) concomitant use of NSAIDs can be restricted; and/or d) can include robust radiologic monitoring by: i) excluding patients with a history of RPOA, ON, SIF or other joint arthropathies that would place patients at risk of joint destruction; ii) schedule radiographic surveillance; iii) timely evaluation (clinical assessment/X-rays/MRI) of any reports of worsening pain in any joint; and iv) consider RPOA/SIF/ON as AESI and study drug to be discontinued.

The present disclosure also provides methods of identifying a subject having an increased risk of developing OA. In some embodiments, the method comprises determining or having determined in a biological sample obtained from the subject the presence or absence of a CILP2 variant nucleic acid molecule (such as a genomic nucleic acid molecule, mRNA molecule, and/or cDNA molecule). When the subject lacks a CILP2 variant nucleic acid molecule (i.e., the subject is genotypically categorized as CILP2 reference), then the subject has an increased risk of developing OA. When the subject has a CILP2 variant nucleic acid molecule (i.e., the subject is heterozygous or homozygous for a CILP2 variant nucleic acid molecule), then the subject has a decreased risk of developing OA. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

Having a single copy of a CILP2 variant nucleic acid molecule is more protective of a subject from developing OA than having no copies of a CILP2 variant nucleic acid molecule. Without intending to be limited to any particular theory or mechanism of action, it is believed that a single copy of a CILP2 variant nucleic acid molecule (i.e., heterozygous for a CILP2 variant nucleic acid molecule) is protective of a subject from developing OA and it is also believed that having two copies of a CILP2 variant nucleic acid molecule (i.e., homozygous for a CILP2 variant nucleic acid molecule) may be more protective of a subject from developing OA, relative to a subject with a single copy. Thus, in some embodiments, a single copy of a CILP2 variant nucleic acid molecule may not be completely protective, but instead, may be partially or incompletely protective of a subject from developing OA. While not desiring to be bound by any particular theory, there may be additional factors or molecules involved in the development of OA that are still present in a subject having a single copy of a CILP2 variant nucleic acid molecule, thus resulting in less than complete protection from the development of OA.

Determining whether a subject has a CILP2 variant nucleic acid molecule in a biological sample from a subject and/or determining whether a subject has a CILP2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing OA, the subject is administered an OA therapeutic agent or OA therapy, and/or a CILP2 inhibitor, as described herein. For example, when the subject is CILP2 reference, and therefore has an increased risk of developing OA, the subject is administered an OA therapeutic agent in an amount that is the same as or less than a standard dosage amount or OA therapy, and/or is administered a CILP2 inhibitor. In some embodiments, when the subject is heterozygous for a CILP2 variant nucleic acid molecule, the subject is administered the OA therapeutic agent in an amount that is the same as or less than a standard dosage amount or OA therapy, and/or is administered a CILP2 inhibitor. In some embodiments, when the subject is homozygous for a CILP2 variant nucleic acid molecule, the subject is administered an OA therapeutic agent in a standard dosage amount or OA therapy. In some embodiments, the subject is CILP2 reference. In some embodiments, the subject is heterozygous for a CILP2 variant nucleic acid molecule. In some embodiments, the subject is homozygous for a CILP2 variant nucleic acid molecule.

The present disclosure also provides methods of determining a subject's aggregate burden, or risk score, of having two or more CILP2 variant nucleic acid molecules, and/or two or more CILP2 variant polypeptides associated with a decreased risk of developing OA. The aggregate burden is the sum of two or more genetic variants that can be carried out in an association analysis with OA. In some embodiments, the subject is homozygous for one or more CILP2 variant nucleic acid molecules associated with a decreased risk of developing OA. In some embodiments, the subject is heterozygous for one or more CILP2 variant nucleic acid molecules associated with a decreased risk of developing OA. When the subject has a lower aggregate burden, the subject has an increased risk of developing OA, and the subject is administered or continued to be administered the OA therapeutic agent in an amount that is the same as or less than the standard dosage amount or OA therapy, and/or a CILP2 inhibitor. When the subject has a higher aggregate burden, the subject has a decreased risk of developing OA and the subject is administered or continued to be administered the OA therapeutic agent in a standard dosage amount or OA therapy. The higher the aggregate burden, the lower the risk of developing OA.

In some embodiments, a subject's aggregate burden of having any two or more CILP2 variant nucleic acid molecules represents a weighted sum of a plurality of any of the CILP2 variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the CILP2 gene, where the genetic burden is the number of alleles multiplied by the association estimate with OA or related outcome for each allele (e.g., a weighted polygenic burden score). In some embodiments, when the subject has an aggregate burden higher than a desired threshold score, the subject has a decreased risk of developing OA. In some embodiments, when the subject has an aggregate burden lower than a desired threshold score, the subject has an increased risk of developing OA.

In some embodiments, the aggregate burden may be divided into quintiles, e.g., top quintile, second quintile, intermediate quintile, fourth quintile, and bottom quintile, wherein the top quintile of aggregate burden corresponds to the lowest risk group and the bottom quintile of aggregate burden corresponds to the highest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with OA in the top 10%, top 20%, top 30%, top 40%, or top 50% of p-value range for the association. In some embodiments, each of the identified genetic variants comprise the genetic variants having association with OA with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with OA with p-value of less than 5×10⁻⁸. In some embodiments, the identified genetic variants comprise genetic variants having association with OA in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the bottom decile, quintile, or tertile in a reference population. The threshold of the aggregate burden can be determined on the basis of the nature of the intended practical application and the risk difference that would be considered meaningful for that practical application.

In embodiments where the aggregate burden is determined for CILP2 genetic variants associated with OA, then the aggregate burden represents a subject's risk score for developing OA. In some embodiments, the aggregate burden or risk score includes the CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule. In some embodiments, a subject's aggregate burden can be determined for CILP2 genetic variants associated with OA in combination with additional genetic variants for other genes also associated with OA to produce a polygenic risk score (PRS) for developing OA. In some embodiments, the PRS includes the CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

The present disclosure also provides methods of detecting the presence or absence of a CILP2 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any CILP2 variant nucleic acid molecule, preliminary processing designed to isolate or enrich the biological sample for the genomic DNA can be employed. A variety of techniques may be used for this purpose. When detecting the level of any CILP2 variant nucleic acid molecule, different techniques can be used to enrich the biological sample with mRNA molecules. Various methods to detect the presence or level of an mRNA molecule or the presence of a particular variant genomic DNA locus can be used.

In some embodiments, detecting a CILP2 variant nucleic acid molecule in a subject comprises performing a sequence analysis on a biological sample obtained from the subject to determine whether a CILP2 genomic nucleic acid molecule in the biological sample, and/or a CILP2 mRNA molecule in the biological sample, and/or a CILP2 cDNA molecule produced from an mRNA molecule in the biological sample, is present in the sample. In some embodiments, the methods detect the CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

In some embodiments, the methods of detecting the presence or absence of a CILP2 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a CILP2 genomic nucleic acid molecule or mRNA molecule, and if mRNA, optionally reverse transcribing the mRNA into cDNA. Such assays can comprise, for example determining the identity of these positions of the particular CILP2 nucleic acid molecule. In some embodiments, the method is an in vitro method.

In some embodiments, the determining step, detecting step, or sequence analysis comprises sequencing at least a portion of the nucleotide sequence of the CILP2 genomic nucleic acid molecule, the CILP2 mRNA molecule, or the CILP2 cDNA molecule in the biological sample that comprises a genetic variation compared to the corresponding CILP2 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a CILP2 genomic nucleic acid molecule is analyzed. In some embodiments, only a CILP2 mRNA is analyzed. In some embodiments, only a CILP2 cDNA obtained from the CILP2 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a CILP2 variant genomic sequence, variant mRNA sequence, or variant cDNA sequence and not the corresponding CILP2 reference sequence under stringent conditions and determining whether hybridization has occurred.

In some embodiments, the determining step, detecting step, or sequence analysis comprises: a) amplifying at least a portion of the CILP2 nucleic acid molecule that encodes the CILP2 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CILP2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to CILP2 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CILP2 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the CILP2 variant nucleic acid molecules disclosed herein. The primers described herein can be used to amplify any CILP2 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding a CILP2 reference genomic nucleic acid molecule, a CILP2 reference mRNA molecule, and/or a CILP2 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides OA therapeutic agents that treat, prevent, or inhibit OA for use in the treatment or prevention of OA in a subject having a CILP2 variant nucleic acid molecule. Any of the OA therapeutic agents that treat, prevent, or inhibit OA described herein can be used herein. Any of the CILP2 variant nucleic acid molecules disclosed herein can be used herein. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

The present disclosure also provides OA therapeutic agents that treat, prevent, or inhibit OA for use in the preparation of a medicament for treating or preventing OA in a subject having a CILP2 variant nucleic acid molecule. Any of the OA therapeutic agents that treat, prevent, or inhibit OA described herein can be used herein. Any of the CILP2 variant nucleic acid molecules disclosed herein can be used herein. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

The present disclosure also provides CILP2 inhibitors for use in the treatment or prevention of OA in a subject that is CILP2 reference or is heterozygous for a CILP2 variant nucleic acid molecule. Any of the CILP2 inhibitors described herein can be used herein. Any of the CILP2 variant nucleic acid molecules disclosed herein can be used herein. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

The present disclosure also provides CILP2 inhibitors for use in the preparation of a medicament for treating or preventing OA in a subject that is CILP2 reference or is heterozygous for a CILP2 variant nucleic acid molecule. Any of the CILP2 inhibitors described herein can be used herein. Any of the CILP2 variant nucleic acid molecules disclosed herein can be used herein. In some embodiments, the CILP2 variant nucleic acid molecule is a CILP2 variant genomic nucleic acid molecule that comprises any one or more of the genetic variations in the genomic nucleic acid molecule (referring to the chromosome: positions set forth in the GRCh38/hg38 human genome assembly) set forth in Table 1, or an mRNA molecule produced therefrom, or a cDNA molecule produced from the mRNA molecule.

In some embodiments, the CILP2 inhibitor and the OA therapeutic agent are disposed within a pharmaceutical composition. In some embodiments, the CILP2 inhibitor is disposed within a first pharmaceutical composition and the OA therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methods

### Participating Cohorts

Genetic association studies were performed in the United Kingdom Biobank (UKB) cohort, the DiscoverEHR cohort from the Geisinger Health System (GHS) MyCode Community Health Initiative, Mount Sinai BioMe Biobank cohort (SINAI), The University of Pennsylvania Penn Medicine BioBank (UPENN-PMBB), Malmo Diet and Cancer Study (MDCS). UKB is a population-based cohort study of people aged between 40 and 69 years recruited through 22 testing centers in the UK between 2006-2010 (Sudlow et al., PLOS Med., 2015, 12, e1001779). Over 430,000 European ancestry participants from UKB with available whole-exome sequencing and clinical phenotype data were included. The GHS MyCode study Community Health Initiative is a health system-based cohort of patients from Central and Eastern Pennsylvania (USA) recruited in 2007-2019 (Carey et al., Genet Med., 2016, 18, 906-13). Over 130,000 European ancestry participants from GHS with available whole-exome sequencing and clinical phenotype data were included. SINAI is a health-system based cohort of patients from the Mount Sinai BioMe Biobank, recruited from the Mount Sinai Health System (Abul-Husn et al., Cell, 2019, 177, 58-69). UPENN-PMBB is a health-system based cohort of patients from the University of Pennsylvania Penn Medicine BioBank (Park et al., Genet. Med., 2020, 22, 102-111). MDCS is a Swedish population-based, prospective, observational cohort recruited between 1991 and 1996 (Berglund et al., J. Intern. Med., 1993, 233, 45-51).

### Phenotype Definitions

Osteoarthritis cases were adjudicated in each cohort on the basis of one or more of the following criteria: 1) an electronic health record history of osteoarthritis (using International Classification of Diseases, Tenth Revision [ICD-10] diagnosis codes M15, M16, M17, M18, M19, M47 or corresponding Ninth Revision [ICD-9] codes), in at least one inpatient encounter or at least two outpatient encounters or if noted as a cause of death; 2) an electronic health record or self-reported history of surgical procedures relating to knee and/or hip replacement; 3) a self-reported physician diagnosis of osteoarthritis. Individuals not meeting any of the criteria for osteoarthritis were used as controls. In addition, individuals were excluded from the control group if they had an electronic health record or self-reported history of other forms of arthritis or joint-related symptoms, or a surgical procedure involving a joint.

An imaging phenotype was derived from dual x-ray absorptiometry (DXA) knee images. D×A images are 2D projections similar to standard x-ray acquisition. The knee bones on D×A images were segmented using a convolutional network algorithm based on the U-Net architecture. The joint space width (JSW) was quantified after measuring the distance between the proximal tibia and the distal femur. The JSW was used as an independent imaging-derived marker of joint health.

### Genotype Data

High coverage whole exome sequencing was performed as previously described (Dewey et al., Science, 2016, 354, aaf6814; Van Hout et al., Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20×coverage over 85% of targeted bases in 96% of VCRome samples and 20×coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods., 2010, 7, 248-9), LRT (Chun et al., Genome Res., 2009, 19, 1553-61) and MutationTaster (Schwarz et al., Nat. Methods., 2010, 7, 575-6). For each gene, the alternative allele frequency (AAF) and functional annotation of each variant determined inclusion into these 7 gene burden exposures: 1) pLOF variants with AAF <1%; 2) pLOF or missense variants predicted deleterious by 5/5 algorithms with AAF <1%; 3) pLOF or missense variants predicted deleterious by 5/5 algorithms with AAF <0.1%; 4) pLOF or missense variants predicted deleterious by at least 1/5 algorithms with AAF <1%; 5) pLOF or missense variants predicted deleterious by at least 1/5 algorithms with AAF <0.1%; 6) pLOF or any missense variants with AAF <1%; 7) pLOF or any missense variants with AAF <0.1%.

Association Analysis of Gene Burden of Rare pLOF and Missense Variation

An association between the burden of rare predicted loss-of-function or missense variants in a given gene and phenotype was analyzed by fitting a linear (for quantitative traits) or firth bias-corrected logistic (for binary traits) regression model adjusted for a polygenic score that approximates a genomic kinship matrix using REGENIE (Mbatchou et al., Nat. Genet., 2021, 53, 1097-1103). Analyses were stratified by ancestry and adjusted for age, sex, age², age-by-sex and age²-by-sex interaction terms, and several cohort-specific covariates including batch-related covariates, and common and rare variant-derived principal components. Results across cohorts for each variant-phenotype association were combined using fixed effect inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association.

### Example 2: Association of Osteaoarthritis and CILP2

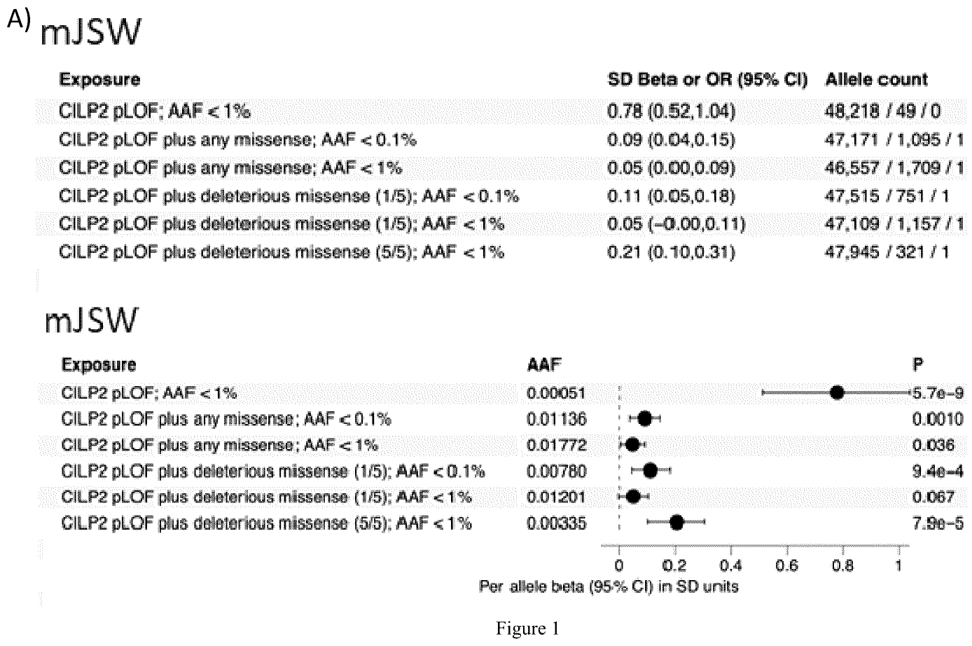

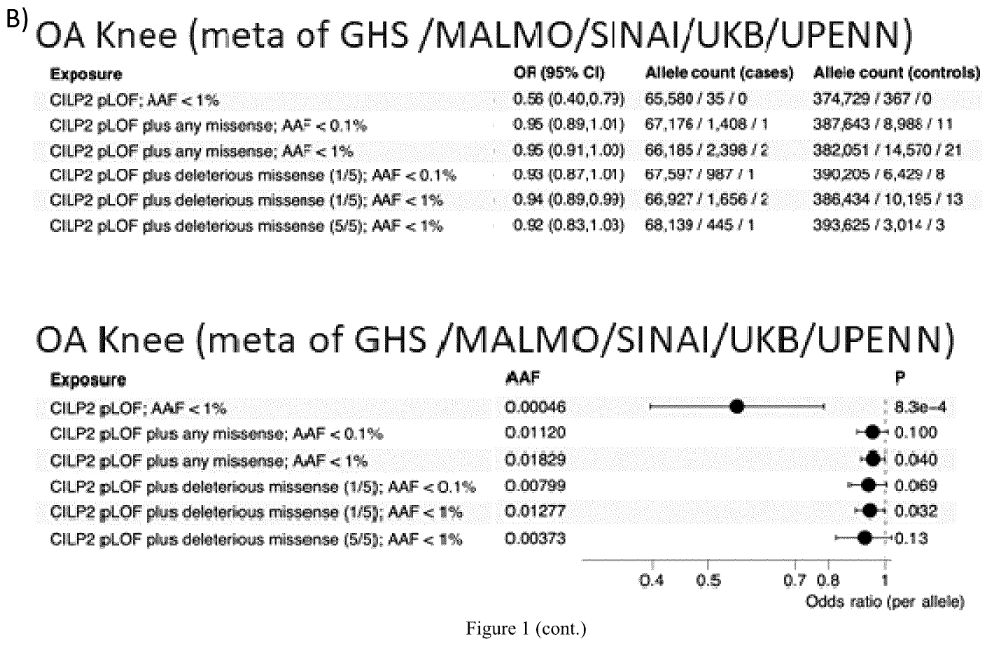

Rare (alternative allele frequency <1%) predicted loss of function (PLOF) variants in CILP2 were observed to be associated with a 0.78 standard deviation (SD) units larger minimum joint space width at the knee (mJSW; p-value=5.7e−9; FIG. 1A). CILP2 rare pLOF variants were also associated with a 44% lower risk of osteoarthritis involving the knee (odds ratio 0.56; p-value=8.3e−4; FIG. 1B). Analyses including pLOF and missense variants were consistent with the pLOF-only analysis. These data indicate that inhibition or loss of CILP2 may lead to greater mJSW and reduce the risk of OA.

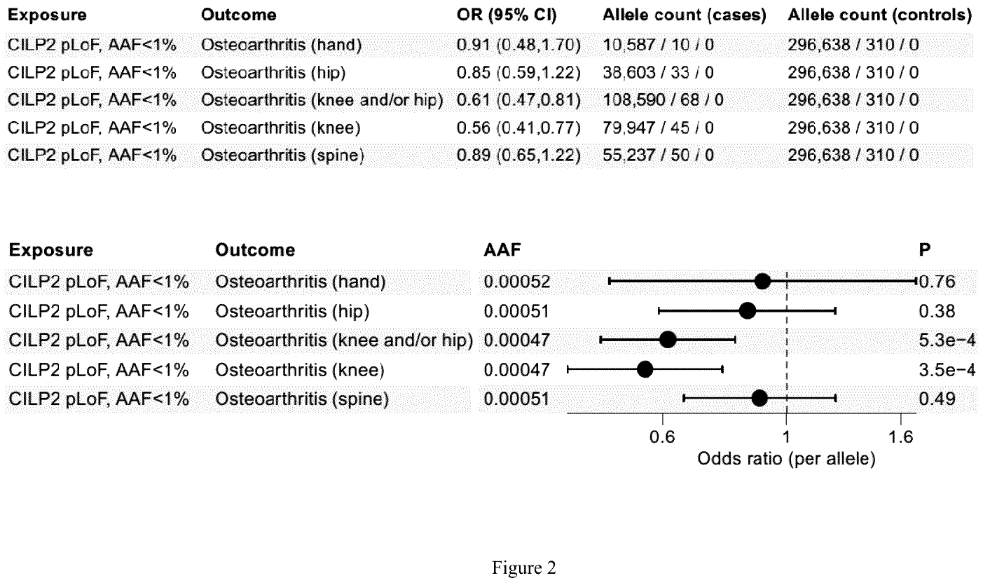

Rare pLOF variants in CILP2 were also observed to be associated with lower risk of knee OA (odds ratio 0.57; p-value=7.5e−4) and lower risk of knee and/or hip OA (odds ratio 0.62; p-value=9.1e−4) (see, FIG. 2).

Table 1 includes pLoF and missense variants detected via exome sequencing of CILP2. CPRA indicates the genomic coordinates: chromosome, physical genomic position in base pairs, reference allele, and alternative allele for each variant, according to build 38 of the Human Genome sequence by the Human Genome Reference Consortium. The HGVS.p and HGVS.c columns indicate protein and coding DNA changes according to the Human Genome Variation Society nomenclature. Effect on transcript column indicates predicted impact of variants.

TABLE 1

| CPRA | HGVS.p | HGVS.c | Effect on Transcript |
|---|---|---|---|
| 19:19538351:T:C | ENST00000291495.5:c.2T > C | ENSP00000291495.3:p.Met1? | start_lost |
| 19:19538359:C:G | ENST00000291495.5:c.10C > G | ENSP00000291495.3:p.Leu4Val | missense |
| 19:19538360:T:C | ENST00000291495.5:c.11T > C | ENSP00000291495.3:p.Leu4Pro | missense |
| 19:19538361:G:GA | ENST00000291495.5:c.12_13insA | ENSP00000291495.3:p.Leu5ThrfsTer198 | frameshift |
| 19:19538365:C:T | ENST00000291495.5:c.16C > T | ENSP00000291495.3:p.Pro6Ser | missense |
| 19:19538369:T:C | ENST00000291495.5:c.20T > C | ENSP00000291495.3:p.Leu7Pro | missense |
| 19:19538371:C:A | ENST00000291495.5:c.22C > A | ENSP00000291495.3:p.Leu8Ile | missense |
| 19:19538371:C:G | ENST00000291495.5:c.22C > G | ENSP00000291495.3:p.Leu8Val | missense |
| 19:19538379:CTG:C | ENST00000291495.5:c.34_35del | ENSP00000291495.3:p.Val12ArgfsTer190 | frameshift |
| 19:19538386:G:T | ENST00000291495.5:c.37G > T | ENSP00000291495.3:p.Val13Phe | missense |
| 19:19538389:G:A | ENST00000291495.5:c.40G > A | ENSP00000291495.3:p.Ala14Thr | missense |
| 19:19538397:C:G | ENST00000291495.5:c.48C > G | ENSP00000291495.3:p.His16Gln | missense |
| 19:19538402:C:A | ENST00000291495.5:c.53C > A | ENSP00000291495.3:p.Ala18Glu | missense |
| 19:19538407:G:A | ENST00000291495.5:c.58G > A | ENSP00000291495.3:p.Ala20Thr | missense |
| 19:19538408:C:T | ENST00000291495.5:c.59C > T | ENSP00000291495.3:p.Ala20Val | missense |
| 19:19538411:G:A | ENST00000291495.5:c.62G > A | ENSP00000291495.3:p.Arg21Gln | missense |
| 19:19538413:G:A | ENST00000291495.5:c.64G > A | ENSP00000291495.3:p.Asp22Asn | missense |
| 19:19538414:G:A | ENST00000291495.5:c.64 + 1G > A | NA | splice_donor |
| 19:19539679:A:T | ENST00000291495.5:c.65A > T | ENSP00000291495.3:p.Asp22Val | missense |
| 19:19539680:C:G | ENST00000291495.5:c.66C > G | ENSP00000291495.3:p.Asp22Glu | missense |
| 19:19539681:G:A | ENST00000291495.5:c.67G > A | ENSP00000291495.3:p.Ala23Thr | missense |
| 19:19539682:C:T | ENST00000291495.5:c.68C > T | ENSP00000291495.3:p.Ala23Val | missense |
| 19:19539685:C:A | ENST00000291495.5:c.71C > A | ENSP00000291495.3:p.Thr24Asn | missense |
| 19:19539687:C:G | ENST00000291495.5:c.73C > G | ENSP00000291495.3:p.Pro25Ala | missense |
| 19:19539688:C:T | ENST00000291495.5:c.74C > T | ENSP00000291495.3:p.Pro25Leu | missense |
| 19:19539690:A:C | ENST00000291495.5:c.76A > C | ENSP00000291495.3:p.Thr26Pro | missense |
| 19:19539693:G:A | ENST00000291495.5:c.79G > A | ENSP00000291495.3:p.Glu27Lys | missense |
| 19:19539694:A:G | ENST00000291495.5:c.80A > G | ENSP00000291495.3:p.Glu27Gly | missense |
| 19:19539696:G:C | ENST00000291495.5:c.82G > C | ENSP00000291495.3:p.Glu28Gln | missense |
| 19:19539703:T:C | ENST00000291495.5:c.89T > C | ENSP00000291495.3:p.Met30Thr | missense |
| 19:19539717:G:A | ENST00000291495.5:c.103G > A | ENSP00000291495.3:p.Gly35Ser | missense |
| 19:19539730:G:A | ENST00000291495.5:c.116G > A | ENSP00000291495.3:p.Arg39Gln | missense |
| 19:19539730:G:C | ENST00000291495.5:c.116G > C | ENSP00000291495.3:p.Arg39Pro | missense |
| 19:19539732:T:C | ENST00000291495.5:c.118T > C | ENSP00000291495.3:p.Ser40Pro | missense |
| 19:19539741:AC:A | ENST00000291495.5:c.129del | ENSP00000291495.3:p.Gly44AlafsTer72 | frameshift |
| 19:19539744:G:A | ENST00000291495.5:c.130G > A | ENSP00000291495.3:p.Gly44Ser | missense |
| 19:19539747:C:A | ENST00000291495.5:c.133C > A | ENSP00000291495.3:p.Gln45Lys | missense |
| 19:19539756:C:G | ENST00000291495.5:c.142C > G | ENSP00000291495.3:p.Pro48Ala | missense |
| 19:19539760:C:A | ENST00000291495.5:c.146C > A | ENSP00000291495.3:p.Ala49Asp | missense |
| 19:19539760:C:T | ENST00000291495.5:c.146C > T | ENSP00000291495.3:p.Ala49Val | missense |
| 19:19539772:G:C | ENST00000291495.5:c.158G > C | ENSP00000291495.3:p.Trp53Ser | missense |
| 19:19539773:G:A | ENST00000291495.5:c.159G > A | ENSP00000291495.3:p.Trp53Ter | stop_gained |
| 19:19539774:G:C | ENST00000291495.5:c.160G > C | ENSP00000291495.3:p.Glu54Gln | missense |
| 19:19539777:G:GGTGA | ENST00000291495.5:c.163 + 3_163 + 6dup | NA | frameshift |
| 19:19539778:G:T | ENST00000291495.5:c.163 + 1G > T | NA | splice_donor |
| 19:19540203:G:C | ENST00000291495.5:c.164 − 1G > C | NA | splice_acceptor |
| 19:19540205:G:T | ENST00000291495.5:c.165G > T | ENSP00000291495.3:p.Glu55Asp | missense |
| 19:19540211:C:G | ENST00000291495.5:c.171C > G | ENSP00000291495.3:p.Ser57Arg | missense |
| 19:19540212:G:C | ENST00000291495.5:c.172G > C | ENSP00000291495.3:p.Glu58Gln | missense |
| 19:19540221:T:A | ENST00000291495.5:c.181T > A | ENSP00000291495.3:p.Ser61Thr | missense |
| 19:19540228:T:A | ENST00000291495.5:c.188T > A | ENSP00000291495.3:p.Phe63Tyr | missense |
| 19:19540233:G:A | ENST00000291495.5:c.193G > A | ENSP00000291495.3:p.Val65Met | missense |
| 19:19540233:G:C | ENST00000291495.5:c.193G > C | ENSP00000291495.3:p.Val65Leu | missense |
| 19:19540238:C:G | ENST00000291495.5:c.198C > G | ENSP00000291495.3:p.Asp66Glu | missense |
| 19:19540239:C:A | ENST00000291495.5:c.199C > A | ENSP00000291495.3:p.His67Asn | missense |
| 19:19540243:C:G | ENST00000291495.5:c.203C > G | ENSP00000291495.3:p.Pro68Arg | missense |
| 19:19540249:G:A | ENST00000291495.5:c.209G > A | ENSP00000291495.3:p.Gly70Asp | missense |
| 19:19540254:G:A | ENST00000291495.5:c.214G > A | ENSP00000291495.3:p.Gly72Ser | missense |
| 19:19540254:G:C | ENST00000291495.5:c.214G > C | ENSP00000291495.3:p.Gly72Arg | missense |
| 19:19540258:A:T | ENST00000291495.5:c.218A > T | ENSP00000291495.3:p.Asp73Val | missense |
| 19:19540260:T:A | ENST00000291495.5:c.220T > A | ENSP00000291495.3:p.Phe74Ile | missense |
| 19:19540267:G:A | ENST00000291495.5:c.227G > A | ENSP00000291495.3:p.Ser76Asn | missense |
| 19:19540272:G:A | ENST00000291495.5:c.232G > A | ENSP00000291495.3:p.Ala78Thr | missense |
| 19:19540276:C:T | ENST00000291495.5:c.236C > T | ENSP00000291495.3:p.Ala79Val | missense |
| 19:19540281:C:A | ENST00000291495.5:c.241C > A | ENSP00000291495.3:p.Arg81Ser | missense |
| 19:19540281:C:G | ENST00000291495.5:c.241C > G | ENSP00000291495.3:p.Arg81Gly | missense |
| 19:19540281:C:T | ENST00000291495.5:c.241C > T | ENSP00000291495.3:p.Arg81Cys | missense |
| 19:19540284:T:C | ENST00000291495.5:c.244T > C | ENSP00000291495.3:p.Phe82Leu | missense |
| 19:19540286:C:A | ENST00000291495.5:c.246C > A | ENSP00000291495.3:p.Phe82Leu | missense |
| 19:19540287:T:C | ENST00000291495.5:c.247T > C | ENSP00000291495.3:p.Tyr83His | missense |
| 19:19540287:T:G | ENST00000291495.5:c.247T > G | ENSP00000291495.3:p.Tyr83Asp | missense |
| 19:19540293:G:A | ENST00000291495.5:c.253G > A | ENSP00000291495.3:p.Gly85Arg | missense |
| 19:19540294:G:C | ENST00000291495.5:c.254G > C | ENSP00000291495.3:p.Gly85Ala | missense |
| 19:19540303:G:A | ENST00000291495.5:c.263G > A | ENSP00000291495.3:p.Arg88His | missense |
| 19:19540304:C:CGTGT | ENST00000291495.5:c.266_269dup | ENSP00000291495.3:p.Pro91ValfsTer113 | frameshift |
| 19:19540305:G:A | ENST00000291495.5:c.265G > A | ENSP00000291495.3:p.Val89Met | missense |
| 19:19540306:T:G | ENST00000291495.5:c.266T > G | ENSP00000291495.3:p.Val89Gly | missense |
| 19:19540312:C:T | ENST00000291495.5:c.272C > T | ENSP00000291495.3:p.Pro91Leu | missense |
| 19:19540315:G:A | ENST00000291495.5:c.275G > A | ENSP00000291495.3:p.Arg92Glr | missense |
| 19:19540318:C:A | ENST00000291495.5:c.278C > A | ENSP00000291495.3:p.Pro93Glr | missense |
| 19:19540320:C:G | ENST00000291495.5:c.280C > G | ENSP00000291495.3:p.Leu94Val | missense |
| 19:19540321:T:G | ENST00000291495.5:c.281T > G | ENSP00000291495.3:p.Leu94Arg | missense |
| 19:19540330:A:G | ENST00000291495.5:c.290A > G | ENSP00000291495.3:p.Glu97Gly | missense |
| 19:19540336:G:A | ENST00000291495.5:c.296G > A | ENSP00000291495.3:p.Arg99His | missense |
| 19:19540336:G:T | ENST00000291495.5:c.296G > T | ENSP00000291495.3:p.Arg99Leu | missense |
| 19:19540339:C:A | ENST00000291495.5:c.299C > A | ENSP00000291495.3:p.Thr100Asn | missense |
| 19:19540340:CA:C | ENST00000291495.5:c.301del | ENSP00000291495.3:p.Thr101ArgfsTer15 | frameshift |
| 19:19540346:C:A | ENST00000291495.5:c.306C > A | ENSP00000291495.3:p.Asp102Glu | missense |
| 19:19540350:G:A | ENST00000291495.5:c.310G > A | ENSP00000291495.3:p.Ala104Thr | missense |
| 19:19540356:C:T | ENST00000291495.5:c.316C > T | ENSP00000291495.3:p.Pro106Ser | missense |
| 19:19540357:C:T | ENST00000291495.5:c.317C > T | ENSP00000291495.3:p.Pro106Leu | missense |
| 19:19540360:C:A | ENST00000291495.5:c.320C > A | ENSP00000291495.3:p.Ser107Tyr | missense |
| 19:19540365:G:A | ENST00000291495.5:c.325G > A | ENSP00000291495.3:p.Val109Ile | missense |
| 19:19540369:G:A | ENST00000291495.5:c.329G > A | ENSP00000291495.3:p.Gly110Asp | missense |
| 19:19540371:G:A | ENST00000291495.5:c.331G > A | ENSP00000291495.3:p.Glu111Lys | missense |
| 19:19540374:C:A | ENST00000291495.5:c.334C > A | ENSP00000291495.3:p.Arg112Ser | missense |
| 19:19540374:C:T | ENST00000291495.5:c.334C > T | ENSP00000291495.3:p.Arg112Cys | missense |
| 19:19540375:G:A | ENST00000291495.5:c.335G > A | ENSP00000291495.3:p.Arg112His | missense |
| 19:19540377:G:A | ENST00000291495.5:c.337G > A | ENSP00000291495.3:p.Val113Met | missense |
| 19:19540377:G:T | ENST00000291495.5:c.337G > T | ENSP00000291495.3:p.Val113Leu | missense |
| 19:19540381:A:C | ENST00000291495.5:c.341A > C | ENSP00000291495.3:p.His114Pro | missense |
| 19:19540387:A:G | ENST00000291495.5:c.347A > G | ENSP00000291495.3:p.Asn116Ser | missense |
| 19:19540387:A:T | ENST00000291495.5:c.347A > T | ENSP00000291495.3:p.Asn116Ile | missense |
| 19:19540390:C:T | ENST00000291495.5:c.350C > T | ENSP00000291495.3:p.Pro117Leu | missense |
| 19:19540396:G:A | ENST00000291495.5:c.356G > A | ENSP00000291495.3:p.Arg119His | missense |
| 19:19540402:T:A | ENST00000291495.5:c.362T > A | ENSP00000291495.3:p.Phe121Tyr | missense |
| 19:19540402:T:G | ENST00000291495.5:c.362T > G | ENSP00000291495.3:p.Phe121Cys | missense |
| 19:19540404:T:G | ENST00000291495.5:c.364T > G | ENSP00000291495.3:p.Trp122Gly | missense |
| 19:19540406:GTGCC:G | ENST00000291495.5:c.368_371del | ENSP00000291495.3:p.Cys123SerfsTer22 | frameshift |
| 19:19540411:T:TCAACCGCGAG | ENST00000291495.5:c.380_389dup | ENSP00000291495.3:p.Gly131AlafsTer75 | frameshift |
| 19:19540417:G:T | ENST00000291495.5:c.377G > T | ENSP00000291495.3:p.Arg126Leu | missense |
| 19:19540419:G:T | ENST00000291495.5:c.379G > T | ENSP00000291495.3:p.Glu127Ter | stop_gained |
| 19:19540422:C:T | ENST00000291495.5:c.382C > T | ENSP00000291495.3:p.Gln128Ter | stop_gained |
| 19:19540426:C:G | ENST00000291495.5:c.386C > G | ENSP00000291495.3:p.Pro129Arg | missense |
| 19:19540429:G:A | ENST00000291495.5:c.389G > A | ENSP00000291495.3:p.Arg130His | missense |
| 19:19540429:G:T | ENST00000291495.5:c.389G > T | ENSP00000291495.3:p.Arg130Leu | missense |
| 19:19540437:C:A | ENST00000291495.5:c.397C > A | ENSP00000291495.3:p.Arg133Ser | missense |
| 19:19540437:C:G | ENST00000291495.5:c.397C > G | ENSP00000291495.3:p.Arg133Gly | missense |
| 19:19540438:G:T | ENST00000291495.5:c.398G > T | ENSP00000291495.3:p.Arg133Leu | missense |
| 19:19540440:T:C | ENST00000291495.5:c.400T > C | ENSP00000291495.3:p.Cys134Arg | missense |
| 19:19540446:A:C | ENST00000291495.5:c.406A > C | ENSP00000291495.3:p.Asn136His | missense |
| 19:19540446:A:G | ENST00000291495.5:c.406A > G | ENSP00000291495.3:p.Asn136Asp | missense |
| 19:19540447:A:C | ENST00000291495.5:c.407A > C | ENSP00000291495.3:p.Asn136Thr | missense |
| 19:19540454:C:G | ENST00000291495.5:c.414C > G | ENSP00000291495.3:p.His138Gln | missense |
| 19:19540455:G:A | ENST00000291495.5:c.415G > A | ENSP00000291495.3:p.Val139Met | missense |
| 19:19540458:C:T | ENST00000291495.5:c.418C > T | ENSP00000291495.3:p.Arg140Cys | missense |
| 19:19540459:G:C | ENST00000291495.5:c.419G > C | ENSP00000291495.3:p.Arg140Pro | missense |
| 19:19540462:T:G | ENST00000291495.5:c.422T > G | ENSP00000291495.3:p.Phe141Cys | missense |
| 19:19540464:C:A | ENST00000291495.5:c.424C > A | ENSP00000291495.3:p.Arg142Ser | missense |
| 19:19540464:C:G | ENST00000291495.5:c.424C > G | ENSP00000291495.3:p.Arg142Gly | missense |
| 19:19540467:T:A | ENST00000291495.5:c.427T > A | ENSP00000291495.3:p.Cys143Ser | missense |
| 19:19540468:G:A | ENST00000291495.5:c.428G > A | ENSP00000291495.3:p.Cys143Tyr | missense |
| 19:19540476:G:A | ENST00000291495.5:c.436G > A | ENSP00000291495.3:p.Glu146Lys | missense |
| 19:19541093:G:T | ENST00000291495.5:c.439G > T | ENSP00000291495.3:p.Ala147Ser | missense |
| 19:19541094:C:A | ENST00000291495.5:c.440C > A | ENSP00000291495.3:p.Ala147Asp | missense |
| 19:19541094:C:G | ENST00000291495.5:c.440C > G | ENSP00000291495.3:p.Ala147Gly | missense |
| 19:19541094:C:T | ENST00000291495.5:c.440C > T | ENSP00000291495.3:p.Ala147Val | missense |
| 19:19541096:T:G | ENST00000291495.5:c.442T > G | ENSP00000291495.3:p.Ser148Ala | missense |
| 19:19541097:C:T | ENST00000291495.5:c.443C > T | ENSP00000291495.3:p.Ser148Leu | missense |
| 19:19541105:G:C | ENST00000291495.5:c.451G > C | ENSP00000291495.3:p.Ala151Pro | missense |
| 19:19541115:C:T | ENST00000291495.5:c.461C > T | ENSP00000291495.3:p.Pro154Leu | missense |
| 19:19541118:G:A | ENST00000291495.5:c.464G > A | ENSP00000291495.3:p.Trp155Ter | stop_gained |
| 19:19541121:G:A | ENST00000291495.5:c.467G > A | ENSP00000291495.3:p.Gly156Asp | missense |
| 19:19541121:G:T | ENST00000291495.5:c.467G > T | ENSP00000291495.3:p.Gly156Val | missense |
| 19:19541124:C:T | ENST00000291495.5:c.470C > T | ENSP00000291495.3:p.Pro157Leu | missense |
| 19:19541128:C:G | ENST00000291495.5:c.474C > G | ENSP00000291495.3:p.Cys158Trp | missense |
| 19:19541130:C:T | ENST00000291495.5:c.476C > T | ENSP00000291495.3:p.Ser159Leu | missense |
| 19:19541132:G:A | ENST00000291495.5:c.478G > A | ENSP00000291495.3:p.Gly160Arg | missense |
| 19:19541136:G:A | ENST00000291495.5:c.482G > A | ENSP00000291495.3:p.Ser161Asn | missense |
| 19:19541136:G:C | ENST00000291495.5:c.482G > C | ENSP00000291495.3:p.Ser161Thr | missense |
| 19:19541137:C:A | ENST00000291495.5:c.483C > A | ENSP00000291495.3:p.Ser161Arg | missense |
| 19:19541142:G:A | ENST00000291495.5:c.488G > A | ENSP00000291495.3:p.Gly163Glu | missense |
| 19:19541144:C:T | ENST00000291495.5:c.490C > T | ENSP00000291495.3:p.Pro164Ser | missense |
| 19:19541148:G:A | ENST00000291495.5:c.494G > A | ENSP00000291495.3:p.Gly165Asp | missense |
| 19:19541150:C:A | ENST00000291495.5:c.496C > A | ENSP00000291495.3:p.Arg166Ser | missense |
| 19:19541150:C:G | ENST00000291495.5:c.496C > G | ENSP00000291495.3:p.Arg166Gly | missense |
| 19:19541150:C:T | ENST00000291495.5:c.496C > T | ENSP00000291495.3:p.Arg166Cys | missense |
| 19:19541154:G:C | ENST00000291495.5:c.500G > C | ENSP00000291495.3:p.Arg167Pro | missense |
| 19:19541162:C:T | ENST00000291495.5:c.508C > T | ENSP00000291495.3:p.Arg170Cys | missense |
| 19:19541165:C:A | ENST00000291495.5:c.511C > A | ENSP00000291495.3:p.Arg171Ser | missense |
| 19:19541175:C:G | ENST00000291495.5:c.521C > G | ENSP00000291495.3:p.Pro174Arg | missense |
| 19:19541175:C:T | ENST00000291495.5:c.521C > T | ENSP00000291495.3:p.Pro174Leu | missense |
| 19:19541178:GC:G | ENST00000291495.5:c.528del | ENSP00000291495.3:p.Ala177LeufsTer48 | frameshift |
| 19:19541183:G:A | ENST00000291495.5:c.529G > A | ENSP00000291495.3:p.Ala177Thr | missense |
| 19:19541183:G:T | ENST00000291495.5:c.529G > T | ENSP00000291495.3:p.Ala177Ser | missense |
| 19:19541184:C:T | ENST00000291495.5:c.530C > T | ENSP00000291495.3:p.Ala177Val | missense |
| 19:19541185:TG:T | ENST00000291495.5:c.535del | ENSP00000291495.3:p.Asp179MetfsTer46 | frameshift |
| 19:19541187:G:A | ENST00000291495.5:c.533G > A | ENSP00000291495.3:p.Gly178Glu | missense |
| 19:19541198:C:T | ENST00000291495.5:c.544C > T | ENSP00000291495.3:p.Pro182Ser | missense |
| 19:19541204:C:A | ENST00000291495.5:c.550C > A | ENSP00000291495.3:p.Arg184Ser | missense |
| 19:19541216:G:C | ENST00000291495.5:c.562G > C | ENSP00000291495.3:p.Ala188Pro | missense |
| 19:19541216:G:T | ENST00000291495.5:c.562G > T | ENSP00000291495.3:p.Ala188Ser | missense |
| 19:19541219:C:G | ENST00000291495.5:c.565C > G | ENSP00000291495.3:p.Gln189Glu | missense |
| 19:19541223:A:T | ENST00000291495.5:c.569A > T | ENSP00000291495.3:p.Lys190Met | missense |
| 19:19541228:G:C | ENST00000291495.5:c.574G > C | ENSP00000291495.3:p.Val192Leu | missense |
| 19:19541231:C:T | ENST00000291495.5:c.577C > T | ENSP00000291495.3:p.Arg193Trp | missense |
| 19:19541231:CG:C | ENST00000291495.5:c.579del | ENSP00000291495.3:p.Pro194LeufsTer31 | frameshift |
| 19:19541232:G:A | ENST00000291495.5:c.578G > A | ENSP00000291495.3:p.Arg193Gln | missense |
| 19:19541237:C:G | ENST00000291495.5:c.583C > G | ENSP00000291495.3:p.Arg195Gly | missense |
| 19:19541238:G:A | ENST00000291495.5:c.584G > A | ENSP00000291495.3:p.Arg195Gln | missense |
| 19:19541243:C:A | ENST00000291495.5:c.589C > A | ENSP00000291495.3:p.Pro197Thr | missense |
| 19:19541244:C:A | ENST00000291495.5:c.590C > A | ENSP00000291495.3:p.Pro197Gln | missense |
| 19:19541244:C:T | ENST00000291495.5:c.590C > T | ENSP00000291495.3:p.Pro197Leu | missense |
| 19:19541246:G:T | ENST00000291495.5:c.592G > T | ENSP00000291495.3:p.Gly198Trp | missense |
| 19:19541247:G:T | ENST00000291495.5:c.592 + 1G > T | NA | splice_donor |
| 19:19542386:G:C | ENST00000291495.5:c.604G > C | ENSP00000291495.3:p.Asp202His | missense |
| 19:19542389:A:C | ENST00000291495.5:c.607A > C | ENSP00000291495.3:p.Thr203Pro | missense |
| 19:19542393:G:A | ENST00000291495.5:c.611G > A | ENSP00000291495.3:p.Cys204Tyr | missense |
| 19:19542393:G:T | ENST00000291495.5:c.611G > T | ENSP00000291495.3:p.Cys204Phe | missense |
| 19:19542402:C:G | ENST00000291495.5:c.620C > G | ENSP00000291495.3:p.Pro207Arg | missense |
| 19:19542402:C:T | ENST00000291495.5:c.620C > T | ENSP00000291495.3:p.Pro207Leu | missense |
| 19:19542407:C:G | ENST00000291495.5:c.625C > G | ENSP00000291495.3:p.His209Asp | missense |
| 19:19542407:C:T | ENST00000291495.5:c.625C > T | ENSP00000291495.3:p.His209Tyr | missense |
| 19:19542417:T:C | ENST00000291495.5:c.635T > C | ENSP00000291495.3:p.Leu212Pro | missense |
| 19:19542419:G:A | ENST00000291495.5:c.637G > A | ENSP00000291495.3:p.Gly213Ser | missense |
| 19:19542419:G:T | ENST00000291495.5:c.637G > T | ENSP00000291495.3:p.Gly213Cys | missense |
| 19:19542420:G:A | ENST00000291495.5:c.638G > A | ENSP00000291495.3:p.Gly213Asp | missense |
| 19:19542422:T:A | ENST00000291495.5:c.640T > A | ENSP00000291495.3:p.Ser214Thr | missense |
| 19:19542423:C:T | ENST00000291495.5:c.641C > T | ENSP00000291495.3:p.Ser214Leu | missense |
| 19:19542425:G:C | ENST00000291495.5:c.643G > C | ENSP00000291495.3:p.Val215Leu | missense |
| 19:19542426:T:C | ENST00000291495.5:c.644T > C | ENSP00000291495.3:p.Val215Ala | missense |
| 19:19542432:C:T | ENST00000291495.5:c.650C > T | ENSP00000291495.3:p.Thr217Ile | missense |
| 19:19542445:A:C | ENST00000291495.5:c.663A > C | ENSP00000291495.3:p.Gln221His | missense |
| 19:19542445:A:T | ENST00000291495.5:c.663A > T | ENSP00000291495.3:p.Gln221His | missense |
| 19:19542453:T:C | ENST00000291495.5:c.671T > C | ENSP00000291495.3:p.Leu224Pro | missense |
| 19:19542455:G:A | ENST00000291495.5:c.673G > A | ENSP00000291495.3:p.Gly225Arg | missense |
| 19:19542456:G:A | ENST00000291495.5:c.674G > A | ENSP00000291495.3:p.Gly225Glu | missense |
| 19:19542459:C:T | ENST00000291495.5:c.677C > T | ENSP00000291495.3:p.Ala226Val | missense |
| 19:19542464:G:A | ENST00000291495.5:c.682G > A | ENSP00000291495.3:p.Val228Ile | missense |
| 19:19542468:C:T | ENST00000291495.5:c.686C > T | ENSP00000291495.3:p.Ser229Phe | missense |
| 19:19542473:C:T | ENST00000291495.5:c.691C > T | ENSP00000291495.3:p.Arg231Ter | stop_gained |
| 19:19542474:G:A | ENST00000291495.5:c.692G > A | ENSP00000291495.3:p.Arg231Gln | missense |
| 19:19542476:G:A | ENST00000291495.5:c.694G > A | ENSP00000291495.3:p.Asp232Asn | missense |
| 19:19542477:A:G | ENST00000291495.5:c.695A > G | ENSP00000291495.3:p.Asp232Gly | missense |
| 19:19542485:G:A | ENST00000291495.5:c.703G > A | ENSP00000291495.3:p.Gly235Ser | missense |
| 19:19542485:G:C | ENST00000291495.5:c.703G > C | ENSP00000291495.3:p.Gly235Arg | missense |
| 19:19542488:A:G | ENST00000291495.5:c.706A > G | ENSP00000291495.3:p.Thr236Ala | missense |
| 19:19542491:G:C | ENST00000291495.5:c.709G > C | ENSP00000291495.3:p.Val237Leu | missense |
| 19:19542497:A:G | ENST00000291495.5:c.715A > G | ENSP00000291495.3:p.Thr239Ala | missense |
| 19:19542498:C:G | ENST00000291495.5:c.716C > G | ENSP00000291495.3:p.Thr239Ser | missense |
| 19:19542501:G:A | ENST00000291495.5:c.719G > A | ENSP00000291495.3:p.Ser240Asn | missense |
| 19:19542502:C:G | ENST00000291495.5:c.720C > G | ENSP00000291495.3:p.Ser240Arg | missense |
| 19:19542503:G:A | ENST00000291495.5:c.721G > A | ENSP00000291495.3:p.Asp241Asn | missense |
| 19:19542505:T:G | ENST00000291495.5:c.723T > G | ENSP00000291495.3:p.Asp241Glu | missense |
| 19:19542506:G:A | ENST00000291495.5:c.724G > A | ENSP00000291495.3:p.Ala242Thr | missense |
| 19:19542512:G:A | ENST00000291495.5:c.730G > A | ENSP00000291495.3:p.Gly244Arg | missense |
| 19:19542516:C:A | ENST00000291495.5:c.734C > A | ENSP00000291495.3:p.Thr245Asn | missense |
| 19:19542519:T:C | ENST00000291495.5:c.737T > C | ENSP00000291495.3:p.Phe246Ser | missense |
| 19:19542521:C:T | ENST00000291495.5:c.739C > T | ENSP00000291495.3:p.Arg247Trp | missense |
| 19:19542522:G:A | ENST00000291495.5:c.740G > A | ENSP00000291495.3:p.Arg247Gln | missense |
| 19:19542522:G:C | ENST00000291495.5:c.740G > C | ENSP00000291495.3:p.Arg247Pro | missense |
| 19:19542530:G:A | ENST00000291495.5:c.748G > A | ENSP00000291495.3:p.Gly250Ser | missense |
| 19:19542536:T:C | ENST00000291495.5:c.754T > C | ENSP00000291495.3:p.Cys252Arg | missense |
| 19:19542547:C:G | ENST00000291495.5:c.765C > G | ENSP00000291495.3:p.Ser255Arg | missense |
| 19:19542548:C:T | ENST00000291495.5:c.766C > T | ENSP00000291495.3:p.Arg256Cys | missense |
| 19:19542549:G:A | ENST00000291495.5:c.767G > A | ENSP00000291495.3:p.Arg256His | missense |
| 19:19542551:G:A | ENST00000291495.5:c.769G > A | ENSP00000291495.3:p.Ala257Thr | missense |
| 19:19542552:C:T | ENST00000291495.5:c.770C > T | ENSP00000291495.3:p.Ala257Val | missense |
| 19:19542559:C:G | ENST00000291495.5:c.777C > G | ENSP00000291495.3:p.Ile259Met | missense |
| 19:19542568:G:C | ENST00000291495.5:c.786G > C | ENSP00000291495.3:p.Gln262His | missense |
| 19:19542569:A:G | ENST00000291495.5:c.787A > G | ENSP00000291495.3:p.Met263Val | missense |
| 19:19542570:T:A | ENST00000291495.5:c.788T > A | ENSP00000291495.3:p.Met263Lys | missense |
| 19:19542570:T:C | ENST00000291495.5:c.788T > C | ENSP00000291495.3:p.Met263Thr | missense |
| 19:19542570:T:G | ENST00000291495.5:c.788T > G | ENSP00000291495.3:p.Met263Arg | missense |
| 19:19542571:G:A | ENST00000291495.5:c.789G > A | ENSP00000291495.3:p.Met263Ile | missense |
| 19:19542584:G:T | ENST00000291495.5:c.802G > T | ENSP00000291495.3:p.Ala268Ser | missense |
| 19:19542587:G:A | ENST00000291495.5:c.805G > A | ENSP00000291495.3:p.Gly269Arg | missense |
| 19:19542592:G:T | ENST00000291495.5:c.810G > T | ENSP00000291495.3:p.Glu270Asp | missense |
| 19:19542593:G:A | ENST00000291495.5:c.811G > A | ENSP00000291495.3:p.Ala271Thr | missense |
| 19:19542597:A:G | ENST00000291495.5:c.815A > G | ENSP00000291495.3:p.Gln272Arg | missense |
| 19:19542604:G:T | ENST00000291495.5:c.822G > T | ENSP00000291495.3:p.Gln274His | missense |
| 19:19542605:G:A | ENST00000291495.5:c.823G > A | ENSP00000291495.3:p.Ala275Thr | missense |
| 19:19542606:C:T | ENST00000291495.5:c.824C > T | ENSP00000291495.3:p.Ala275Val | missense |
| 19:19542611:G:A | ENST00000291495.5:c.829G > A | ENSP00000291495.3:p.Gly277Arg | missense |
| 19:19542611:G:T | ENST00000291495.5:c.829G > T | ENSP00000291495.3:p.Gly277Ter | stop_gained |
| 19:19542632:A:T | ENST00000291495.5:c.850A > T | ENSP00000291495.3:p.Ile284Phe | missense |
| 19:19542635:A:C | ENST00000291495.5:c.853A > C | ENSP00000291495.3:p.Ile285Leu | missense |
| 19:19542636:T:C | ENST00000291495.5:c.854T > C | ENSP00000291495.3:p.Ile285Thr | missense |
| 19:19542639:T:C | ENST00000291495.5:c.857T > C | ENSP00000291495.3:p.Leu286Pro | missense |
| 19:19542641:G:A | ENST00000291495.5:c.859G > A | ENSP00000291495.3:p.Asp287Asn | missense |
| 19:19542644:A:C | ENST00000291495.5:c.862A > C | ENSP00000291495.3:p.Lys288Gln | missense |
| 19:19542648:T:G | ENST00000291495.5:c.866T > G | ENSP00000291495.3:p.Leu289Trp | missense |
| 19:19542651:G:A | ENST00000291495.5:c.868 + 1G > A | NA | splice_donor |
| 19:19542868:G:C | ENST00000291495.5:c.873G > C | ENSP00000291495.3:p.Lys291Asn | missense |
| 19:19542869:C:G | ENST00000291495.5:c.874C > G | ENSP00000291495.3:p.Pro292Ala | missense |
| 19:19542878:G:A | ENST00000291495.5:c.883G > A | ENSP00000291495.3:p.Val295Met | missense |
| 19:19542884:C:A | ENST00000291495.5:c.889C > A | ENSP00000291495.3:p.His297Asn | missense |
| 19:19542886:C:A | ENST00000291495.5:c.891C > A | ENSP00000291495.3:p.His297Gln | missense |
| 19:19542887:C:A | ENST00000291495.5:c.892C > A | ENSP00000291495.3:p.Pro298Thr | missense |
| 19:19542896:C:T | ENST00000291495.5:c.901C > T | ENSP00000291495.3:p.Arg301Ter | stop_gained |
| 19:19542897:G:A | ENST00000291495.5:c.902G > A | ENSP00000291495.3:p.Arg301Gln | missense |
| 19:19542899:G:C | ENST00000291495.5:c.904G > C | ENSP00000291495.3:p.Val302Leu | missense |
| 19:19542900:T:C | ENST00000291495.5:c.905T > C | ENSP00000291495.3:p.Val302Ala | missense |
| 19:19542902:C:G | ENST00000291495.5:c.907C > G | ENSP00000291495.3:p.Arg303Gly | missense |
| 19:19542902:C:T | ENST00000291495.5:c.907C > T | ENSP00000291495.3:p.Arg303Ter | stop_gained |
| 19:19542903:G:A | ENST00000291495.5:c.908G > A | ENSP00000291495.3:p.Arg303Gln | missense |
| 19:19542907:G:T | ENST00000291495.5:c.912G > T | ENSP00000291495.3:p.Glu304Asp | missense |
| 19:19542908:G:T | ENST00000291495.5:c.913G > T | ENSP00000291495.3:p.Ala305Ser | missense |
| 19:19542914:C:T | ENST00000291495.5:c.919C > T | ENSP00000291495.3:p.Gln307Ter | stop_gained |
| 19:19542915:A:G | ENST00000291495.5:c.920A > G | ENSP00000291495.3:p.Gln307Arg | missense |
| 19:19542918:A:C | ENST00000291495.5:c.923A > C | ENSP00000291495.3:p.Asn308Thr | missense |
| 19:19542923:A:G | ENST00000291495.5:c.928A > G | ENSP00000291495.3:p.Thr310Ala | missense |
| 19:19542923:AC:A | ENST00000291495.5:c.929del | ENSP00000291495.3:p.Thr310IlefsTer84 | frameshift |
| 19:19542930:G:C | ENST00000291495.5:c.935G > C | ENSP00000291495.3:p.Cys312Ser | missense |
| 19:19542934:C:A | ENST00000291495.5:c.939C > A | ENSP00000291495.3:p.Cys313Ter | stop_gained |
| 19:19542939:C:T | ENST00000291495.5:c.944C > T | ENSP00000291495.3:p.Ala315Val | missense |
| 19:19542944:G:A | ENST00000291495.5:c.949G > A | ENSP00000291495.3:p.Gly317Arg | missense |
| 19:19542948:C:A | ENST00000291495.5:c.953C > A | ENSP00000291495.3:p.Thr318Asn | missense |
| 19:19542948:C:G | ENST00000291495.5:c.953C > G | ENSP00000291495.3:p.Thr318Ser | missense |
| 19:19542950:C:T | ENST00000291495.5:c.955C > T | ENSP00000291495.3:p.Pro319Ser | missense |
| 19:19542956:C:T | ENST00000291495.5:c.961C > T | ENSP00000291495.3:p.Pro321Ser | missense |
| 19:19542963:A:C | ENST00000291495.5:c.968A > C | ENSP00000291495.3:p.Lys323Thr | missense |
| 19:19542964:A:C | ENST00000291495.5:c.969A > C | ENSP00000291495.3:p.Lys323Asn | missense |
| 19:19542972:G:A | ENST00000291495.5:c.977G > A | ENSP00000291495.3:p.Trp326Ter | stop_gained |
| 19:19542973:G:A | ENST00000291495.5:c.977 + 1G > A | NA | splice_donor |
| 19:19543256:A:G | ENST00000291495.5:c.986A > G | ENSP00000291495.3:p.Asn329Ser | missense |
| 19:19543256:A:T | ENST00000291495.5:c.986A > T | ENSP00000291495.3:p.Asn329Ile | missense |
| 19:19543258:G:A | ENST00000291495.5:c.988G > A | ENSP00000291495.3:p.Gly330Arg | missense |
| 19:19543259:G:A | ENST00000291495.5:c.989G > A | ENSP00000291495.3:p.Gly330Glu | missense |
| 19:19543267:C:G | ENST00000291495.5:c.997C > G | ENSP00000291495.3:p.Leu333Val | missense |
| 19:19543274:G:A | ENST00000291495.5:c.1004G > A | ENSP00000291495.3:p.Arg335Lys | missense |
| 19:19543276:C:T | ENST00000291495.5:c.1006C > T | ENSP00000291495.3:p.Arg336Ter | stop_gained |
| 19:19543277:G:A | ENST00000291495.5:c.1007G > A | ENSP00000291495.3:p.Arg336Gln | missense |
| 19:19543279:G:C | ENST00000291495.5:c.1009G > C | ENSP00000291495.3:p.Ala337Pro | missense |
| 19:19543283:A:C | ENST00000291495.5:c.1013A > C | ENSP00000291495.3:p.His338Pro | missense |
| 19:19543283:A:T | ENST00000291495.5:c.1013A > T | ENSP00000291495.3:p.His338Leu | missense |
| 19:19543286:G:C | ENST00000291495.5:c.1016G > C | ENSP00000291495.3:p.Gly339Ala | missense |
| 19:19543288:T:C | ENST00000291495.5:c.1018T > C | ENSP00000291495.3:p.Tyr340His | missense |
| 19:19543291:G:A | ENST00000291495.5:c.1021G > A | ENSP00000291495.3:p.Gly341Arg | missense |
| 19:19543294:G:A | ENST00000291495.5:c.1024G > A | ENSP00000291495.3:p.Ala342Thr | missense |
| 19:19543295:C:G | ENST00000291495.5:c.1025C > G | ENSP00000291495.3:p.Ala342Gly | missense |
| 19:19543297:C:A | ENST00000291495.5:c.1027C > A | ENSP00000291495.3:p.His343Asn | missense |
| 19:19543300:C:G | ENST00000291495.5:c.1030C > G | ENSP00000291495.3:p.Leu344Val | missense |
| 19:19543305:G:T | ENST00000291495.5:c.1035G > T | ENSP00000291495.3:p.Glu345Asp | missense |
| 19:19543309:C:T | ENST00000291495.5:c.1039C > T | ENSP00000291495.3:p.Arg347Trp | missense |
| 19:19543310:G:A | ENST00000291495.5:c.1040G > A | ENSP00000291495.3:p.Arg347Gln | missense |
| 19:19543310:G:C | ENST00000291495.5:c.1040G > C | ENSP00000291495.3:p.Arg347Pro | missense |
| 19:19543313:G:A | ENST00000291495.5:c.1043G > A | ENSP00000291495.3:p.Gly348Glu | missense |
| 19:19543313:GA:G | ENST00000291495.5:c.1044del | ENSP00000291495.3:p.Leu349CysfsTer45 | frameshift |
| 19:19543318:C:A | ENST00000291495.5:c.1048C > A | ENSP00000291495.3:p.Arg350Ser | missense |
| 19:19543318:C:T | ENST00000291495.5:c.1048C > T | ENSP00000291495.3:p.Arg350Cys | missense |
| 19:19543319:G:A | ENST00000291495.5:c.1049G > A | ENSP00000291495.3:p.Arg350His | missense |
| 19:19543330:G:T | ENST00000291495.5:c.1060G > T | ENSP00000291495.3:p.Ala354Ser | missense |
| 19:19543336:A:T | ENST00000291495.5:c.1066A > T | ENSP00000291495.3:p.Ile356Phe | missense |
| 19:19543342:C:G | ENST00000291495.5:c.1072C > G | ENSP00000291495.3:p.His358Asp | missense |
| 19:19543345:T:C | ENST00000291495.5:c.1075T > C | ENSP00000291495.3:p.Cys359Arg | missense |
| 19:19543345:T:G | ENST00000291495.5:c.1075T > G | ENSP00000291495.3:p.Cys359Gly | missense |
| 19:19543350:G:C | ENST00000291495.5:c.1080G > C | ENSP00000291495.3:p.Lys360Asn | missense |
| 19:19543355:G:A | ENST00000291495.5:c.1085G > A | ENSP00000291495.3:p.Trp362Ter | stop_gained |
| 19:19543363:G:A | ENST00000291495.5:c.1093G > A | ENSP00000291495.3:p.Ala365Thr | missense |
| 19:19543364:C:T | ENST00000291495.5:c.1094C > T | ENSP00000291495.3:p.Ala365Val | missense |
| 19:19543370:C:T | ENST00000291495.5:c.1100C > T | ENSP00000291495.3:p.Ala367Val | missense |
| 19:19543372:G:A | ENST00000291495.5:c.1102G > A | ENSP00000291495.3:p.Val368Met | missense |
| 19:19543372:G:C | ENST00000291495.5:c.1102G > C | ENSP00000291495.3:p.Val368Leu | missense |
| 19:19543375:C:T | ENST00000291495.5:c.1105C > T | ENSP00000291495.3:p.Arg369Cys | missense |
| 19:19543376:G:A | ENST00000291495.5:c.1106G > A | ENSP00000291495.3:p.Arg369His | missense |
| 19:19543379:C:T | ENST00000291495.5:c.1109C > T | ENSP00000291495.3:p.Ser370Leu | missense |
| 19:19543382:G:A | ENST00000291495.5:c.1112G > A | ENSP00000291495.3:p.Gly371Asp | missense |
| 19:19543390:C:T | ENST00000291495.5:c.1120C > T | ENSP00000291495.3:p.Arg374Trp | missense |
| 19:19543391:G:A | ENST00000291495.5:c.1121G > A | ENSP00000291495.3:p.Arg374Gln | missense |
| 19:19543396:A:G | ENST00000291495.5:c.1126A > G | ENSP00000291495.3:p.Thr376Ala | missense |
| 19:19543403:T:C | ENST00000291495.5:c.1133T > C | ENSP00000291495.3:p.Leu378Pro | missense |
| 19:19543681:C:T | ENST00000291495.5:c.1136C > T | ENSP00000291495.3:p.Ala379Val | missense |
| 19:19543686:G:T | ENST00000291495.5:c.1141G > T | ENSP00000291495.3:p.Gly381Cys | missense |
| 19:19543690:A:G | ENST00000291495.5:c.1145A > G | ENSP00000291495.3:p.Gln382Arg | missense |
| 19:19543695:G:T | ENST00000291495.5:c.1150G > T | ENSP00000291495.3:p.Ala384Ser | missense |
| 19:19543699:G:T | ENST00000291495.5:c.1154G > T | ENSP00000291495.3:p.Cys385Phe | missense |
| 19:19543701:G:A | ENST00000291495.5:c.1156G > A | ENSP00000291495.3:p.Asp386Asn | missense |
| 19:19543701:G:C | ENST00000291495.5:c.1156G > C | ENSP00000291495.3:p.Asp386His | missense |
| 19:19543705:C:A | ENST00000291495.5:c.1160C > A | ENSP00000291495.3:p.Pro387His | missense |
| 19:19543707:C:T | ENST00000291495.5:c.1162C > T | ENSP00000291495.3:p.Arg388Trp | missense |
| 19:19543708:G:A | ENST00000291495.5:c.1163G > A | ENSP00000291495.3:p.Arg388Gln | missense |
| 19:19543708:G:C | ENST00000291495.5:c.1163G > C | ENSP00000291495.3:p.Arg388Pro | missense |
| 19:19543710:C:G | ENST00000291495.5:c.1165C > G | ENSP00000291495.3:p.Pro389Ala | missense |
| 19:19543710:C:T | ENST00000291495.5:c.1165C > T | ENSP00000291495.3:p.Pro389Ser | missense |
| 19:19543711:C:G | ENST00000291495.5:c.1166C > G | ENSP00000291495.3:p.Pro389Arg | missense |
| 19:19543713:C:G | ENST00000291495.5:c.1168C > G | ENSP00000291495.3:p.Arg390Gly | missense |
| 19:19543713:C:T | ENST00000291495.5:c.1168C > T | ENSP00000291495.3:p.Arg390Ter | stop_gained |
| 19:19543714:G:A | ENST00000291495.5:c.1169G > A | ENSP00000291495.3:p.Arg390Gln | missense |
| 19:19543716:G:A | ENST00000291495.5:c.1171G > A | ENSP00000291495.3:p.Glu391Lys | missense |
| 19:19543716:G:C | ENST00000291495.5:c.1171G > C | ENSP00000291495.3:p.Glu391Gln | missense |
| 19:19543716:G:T | ENST00000291495.5:c.1171G > T | ENSP00000291495.3:p.Glu391Ter | stop_gained |
| 19:19543717:A:G | ENST00000291495.5:c.1172A > G | ENSP00000291495.3:p.Glu391Gly | missense |
| 19:19543718:G:T | ENST00000291495.5:c.1173G > T | ENSP00000291495.3:p.Glu391Asp | missense |
| 19:19543726:T:C | ENST00000291495.5:c.1181T > C | ENSP00000291495.3:p.Ile394Thr | missense |
| 19:19543734:C:T | ENST00000291495.5:c.1189C > T | ENSP00000291495.3:p.Pro397Ser | missense |
| 19:19543737:G:T | ENST00000291495.5:c.1192G > T | ENSP00000291495.3:p.Glu398Ter | stop_gained |
| 19:19543738:AG:A | ENST00000291495.5:c.1195del | ENSP00000291495.3:p.Asp399ThrfsTer59 | frameshift |
| 19:19543743:T:C | ENST00000291495.5:c.1198T > C | ENSP00000291495.3:p.Cys400Arg | missense |
| 19:19543749:C:A | ENST00000291495.5:c.1204C > A | ENSP00000291495.3:p.Gln402Lys | missense |
| 19:19543758:A:C | ENST00000291495.5:c.1213A > C | ENSP00000291495.3:p.Ser405Arg | missense |
| 19:19543762:G:T | ENST00000291495.5:c.1217G > T | ENSP00000291495.3:p.Gly406Val | missense |
| 19:19543770:T:C | ENST00000291495.5:c.1225T > C | ENSP00000291495.3:p.Tyr409His | missense |
| 19:19543771:A:G | ENST00000291495.5:c.1226A > G | ENSP00000291495.3:p.Tyr409Cys | missense |
| 19:19543774:T:G | ENST00000291495.5:c.1229T > G | ENSP00000291495.3:p.Leu410Arg | missense |
| 19:19543774:TG:T | ENST00000291495.5:c.1231del | ENSP00000291495.3:p.Asp411MetfsTer47 | frameshift |
| 19:19543779:G:A | ENST00000291495.5:c.1234G > A | ENSP00000291495.3:p.Val412Met | missense |
| 19:19543782:G:T | ENST00000291495.5:c.1237G > T | ENSP00000291495.3:p.Gly413Cys | missense |
| 19:19543788:T:C | ENST00000291495.5:c.1243T > C | ENSP00000291495.3:p.Cys415Arg | missense |
| 19:19543791:C:A | ENST00000291495.5:c.1246C > A | ENSP00000291495.3:p.Pro416Thr | missense |
| 19:19543791:C:T | ENST00000291495.5:c.1246C > T | ENSP00000291495.3:p.Pro416Ser | missense |
| 19:19543794:G:A | ENST00000291495.5:c.1249G > A | ENSP00000291495.3:p.Asp417Asn | missense |
| 19:19543795:A:C | ENST00000291495.5:c.1250A > C | ENSP00000291495.3:p.Asp417Ala | missense |
| 19:19543795:A:G | ENST00000291495.5:c.1250A > G | ENSP00000291495.3:p.Asp417Gly | missense |
| 19:19543800:C:G | ENST00000291495.5:c.1255C > G | ENSP00000291495.3:p.Arg419Gly | missense |
| 19:19543800:C:T | ENST00000291495.5:c.1255C > T | ENSP00000291495.3:p.Arg419Cys | missense |
| 19:19543801:G:A | ENST00000291495.5:c.1256G > A | ENSP00000291495.3:p.Arg419His | missense |
| 19:19543804:G:A | ENST00000291495.5:c.1259G > A | ENSP00000291495.3:p.Cys420Tyr | missense |
| 19:19543807:C:A | ENST00000291495.5:c.1262C > A | ENSP00000291495.3:p.Pro421His | missense |
| 19:19543810:G:C | ENST00000291495.5:c.1265G > C | ENSP00000291495.3:p.Ser422Thr | missense |
| 19:19543815:G:A | ENST00000291495.5:c.1270G > A | ENSP00000291495.3:p.Ala424Thr | missense |
| 19:19543818:G:A | ENST00000291495.5:c.1273G > A | ENSP00000291495.3:p.Gly425Ser | missense |
| 19:19543819:G:T | ENST00000291495.5:c.1274G > T | ENSP00000291495.3:p.Gly425Val | missense |
| 19:19543824:A:G | ENST00000291495.5:c.1279A > G | ENSP00000291495.3:p.Ser427Gly | missense |
| 19:19543827:C:T | ENST00000291495.5:c.1282C > T | ENSP00000291495.3:p.Pro428Ser | missense |
| 19:19543828:C:T | ENST00000291495.5:c.1283C > T | ENSP00000291495.3:p.Pro428Leu | missense |
| 19:19543830:C:A | ENST00000291495.5:c.1285C > A | ENSP00000291495.3:p.Arg429Ser | missense |
| 19:19543830:C:T | ENST00000291495.5:c.1285C > T | ENSP00000291495.3:p.Arg429Cys | missense |
| 19:19543831:G:A | ENST00000291495.5:c.1286G > A | ENSP00000291495.3:p.Arg429His | missense |
| 19:19543831:G:T | ENST00000291495.5:c.1286G > T | ENSP00000291495.3:p.Arg429Leu | missense |
| 19:19543836:G:A | ENST00000291495.5:c.1291G > A | ENSP00000291495.3:p.Gly431Arg | missense |
| 19:19543841:C:G | ENST00000291495.5:c.1296C > G | ENSP00000291495.3:p.Asp432Glu | missense |
| 19:19543842:G:A | ENST00000291495.5:c.1297G > A | ENSP00000291495.3:p.Ala433Thr | missense |
| 19:19543848:T:C | ENST00000291495.5:c.1303T > C | ENSP00000291495.3:p.Ser435Pro | missense |
| 19:19543849:C:G | ENST00000291495.5:c.1304C > G | ENSP00000291495.3:p.Ser435Cys | missense |
| 19:19543851:C:T | ENST00000291495.5:c.1306C > T | ENSP00000291495.3:p.Arg436Cys | missense |
| 19:19543852:G:C | ENST00000291495.5:c.1307G > C | ENSP00000291495.3:p.Arg436Pro | missense |
| 19:19543861:C:T | ENST00000291495.5:c.1316C > T | ENSP00000291495.3:p.Ser439Phe | missense |
| 19:19543866:C:A | ENST00000291495.5:c.1321C > A | ENSP00000291495.3:p.Arg441Ser | missense |
| 19:19543867:G:A | ENST00000291495.5:c.1322G > A | ENSP00000291495.3:p.Arg441His | missense |
| 19:19543867:G:T | ENST00000291495.5:c.1322G > T | ENSP00000291495.3:p.Arg441Leu | missense |
| 19:19543869:C:G | ENST00000291495.5:c.1324C > G | ENSP00000291495.3:p.Arg442Gly | missense |
| 19:19543869:C:T | ENST00000291495.5:c.1324C > T | ENSP00000291495.3:p.Arg442Cys | missense |
| 19:19543870:G:A | ENST00000291495.5:c.1325G > A | ENSP00000291495.3:p.Arg442His | missense |
| 19:19543876:A:G | ENST00000291495.5:c.1331A > G | ENSP00000291495.3:p.Glu444Gly | missense |
| 19:19543878:A:G | ENST00000291495.5:c.1333A > G | ENSP00000291495.3:p.Arg445Gly | missense |
| 19:19543882:G:C | ENST00000291495.5:c.1337G > C | ENSP00000291495.3:p.Arg446Thr | missense |
| 19:19543885:A:G | ENST00000291495.5:c.1340A > G | ENSP00000291495.3:p.Glu447Gly | missense |
| 19:19543894:G:C | ENST00000291495.5:c.1349G > C | ENSP00000291495.3:p.Cys450Ser | missense |
| 19:19543900:G:A | ENST00000291495.5:c.1355G > A | ENSP00000291495.3:p.Gly452Asp | missense |
| 19:19543905:G:A | ENST00000291495.5:c.1360G > A | ENSP00000291495.3:p.Val454Ile | missense |
| 19:19543905:G:C | ENST00000291495.5:c.1360G > C | ENSP00000291495.3:p.Val454Leu | missense |
| 19:19543906:T:C | ENST00000291495.5:c.1361T > C | ENSP00000291495.3:p.Val454Ala | missense |
| 19:19543919:G:T | ENST00000291495.5:c.1374G > T | ENSP00000291495.3:p.Lys458Asn | missense |
| 19:19543920:G:A | ENST00000291495.5:c.1375G > A | ENSP00000291495.3:p.Val459Met | missense |
| 19:19543921:T:C | ENST00000291495.5:c.1376T > C | ENSP00000291495.3:p.Val459Ala | missense |
| 19:19543932:T:C | ENST00000291495.5:c.1387T > C | ENSP00000291495.3:p.Cys463Arg | missense |
| 19:19543952:G:GC | ENST00000291495.5:c.1412dup | ENSP00000291495.3:p.Arg472SerfsTer12 | frameshift |
| 19:19543953:C:T | ENST00000291495.5:c.1408C > T | ENSP00000291495.3:p.Pro470Ser | missense |
| 19:19543954:C:T | ENST00000291495.5:c.1409C > T | ENSP00000291495.3:p.Pro470Leu | missense |
| 19:19543956:C:T | ENST00000291495.5:c.1411C > T | ENSP00000291495.3:p.Pro471Ser | missense |
| 19:19543957:C:G | ENST00000291495.5:c.1412C > G | ENSP00000291495.3:p.Pro471Arg | missense |
| 19:19543957:C:T | ENST00000291495.5:c.1412C > T | ENSP00000291495.3:p.Pro471Leu | missense |
| 19:19543959:C:G | ENST00000291495.5:c.1414C > G | ENSP00000291495.3:p.Arg472Gly | missense |
| 19:19543959:C:T | ENST00000291495.5:c.1414C > T | ENSP00000291495.3:p.Arg472Trp | missense |
| 19:19543960:G:A | ENST00000291495.5:c.1415G > A | ENSP00000291495.3:p.Arg472Gln | missense |
| 19:19543971:C:T | ENST00000291495.5:c.1426C > T | ENSP00000291495.3:p.Arg476Trp | missense |
| 19:19543971:CG:C | ENST00000291495.5:c.1430del | ENSP00000291495.3:p.Gly477AlafsTer46 | frameshift |
| 19:19543972:G:A | ENST00000291495.5:c.1427G > A | ENSP00000291495.3:p.Arg476Gln | missense |
| 19:19543975:GC:G | ENST00000291495.5:c.1432del | ENSP00000291495.3:p.Arg478ValfsTer45 | frameshift |
| 19:19543977:C:G | ENST00000291495.5:c.1432C > G | ENSP00000291495.3:p.Arg478Gly | missense |
| 19:19543977:C:T | ENST00000291495.5:c.1432C > T | ENSP00000291495.3:p.Arg478Cys | missense |
| 19:19543980:G:T | ENST00000291495.5:c.1435G > T | ENSP00000291495.3:p.Val479Phe | missense |
| 19:19543984:T:C | ENST00000291495.5:c.1439T > C | ENSP00000291495.3:p.Val480Ala | missense |
| 19:19543986:G:A | ENST00000291495.5:c.1441G > A | ENSP00000291495.3:p.Ala481Thr | missense |
| 19:19543986:G:C | ENST00000291495.5:c.1441G > C | ENSP00000291495.3:p.Ala481Pro | missense |
| 19:19543989:G:T | ENST00000291495.5:c.1444G > T | ENSP00000291495.3:p.Ala482Ser | missense |
| 19:19543998:G:A | ENST00000291495.5:c.1453G > A | ENSP00000291495.3:p.Gly485Arg | missense |
| 19:19543998:G:C | ENST00000291495.5:c.1453G > C | ENSP00000291495.3:p.Gly485Arg | missense |
| 19:19544001:G:A | ENST00000291495.5:c.1456G > A | ENSP00000291495.3:p.Glu486Lys | missense |
| 19:19544005:C:T | ENST00000291495.5:c.1460C > T | ENSP00000291495.3:p.Pro487Leu | missense |
| 19:19544010:C:G | ENST00000291495.5:c.1465C > G | ENSP00000291495.3:p.Arg489Gly | missense |
| 19:19544010:C:T | ENST00000291495.5:c.1465C > T | ENSP00000291495.3:p.Arg489Cys | missense |
| 19:19544011:G:A | ENST00000291495.5:c.1466G > A | ENSP00000291495.3:p.Arg489His | missense |
| 19:19544011:G:T | ENST00000291495.5:c.1466G > T | ENSP00000291495.3:p.Arg489Leu | missense |
| 19:19544015:C:G | ENST00000291495.5:c.1470C > G | ENSP00000291495.3:p.Phe490Leu | missense |
| 19:19544016:G:A | ENST00000291495.5:c.1471G > A | ENSP00000291495.3:p.Ala491Thr | missense |
| 19:19544017:C:G | ENST00000291495.5:c.1472C > G | ENSP00000291495.3:p.Ala491Gly | missense |
| 19:19544022:A:G | ENST00000291495.5:c.1477A > G | ENSP00000291495.3:p.Ile493Val | missense |
| 19:19544040:C:T | ENST00000291495.5:c.1495C > T | ENSP00000291495.3:p.Pro499Ser | missense |
| 19:19544044:T:C | ENST00000291495.5:c.1499T > C | ENSP00000291495.3:p.Ile500Thr | missense |
| 19:19544049:T:A | ENST00000291495.5:c.1504T > A | ENSP00000291495.3:p.Phe502Ile | missense |
| 19:19544051:C:G | ENST00000291495.5:c.1506C > G | ENSP00000291495.3:p.Phe502Leu | missense |
| 19:19544055:G:A | ENST00000291495.5:c.1510G > A | ENSP00000291495.3:p.Ala504Thr | missense |
| 19:19544058:T:C | ENST00000291495.5:c.1513T > C | ENSP00000291495.3:p.Tyr505His | missense |
| 19:19544061:C:A | ENST00000291495.5:c.1516C > A | ENSP00000291495.3:p.Gln506Lys | missense |
| 19:19544063:G:C | ENST00000291495.5:c.1518G > C | ENSP00000291495.3:p.Gln506His | missense |
| 19:19544067:G:T | ENST00000291495.5:c.1522G > T | ENSP00000291495.3:p.Asp508Tyr | missense |
| 19:19544074:C:A | ENST00000291495.5:c.1529C > A | ENSP00000291495.3:p.Thr510Asn | missense |
| 19:19544077:T:C | ENST00000291495.5:c.1532T > C | ENSP00000291495.3:p.Ile511Thr | missense |
| 19:19544080:A:C | ENST00000291495.5:c.1535A > C | ENSP00000291495.3:p.Glu512Ala | missense |
| 19:19544086:C:T | ENST00000291495.5:c.1541C > T | ENSP00000291495.3:p.Pro514Leu | missense |
| 19:19544089:C:G | ENST00000291495.5:c.1544C > G | ENSP00000291495.3:p.Pro515Arg | missense |
| 19:19544091:T:G | ENST00000291495.5:c.1546T > G | ENSP00000291495.3:p.Ser516Ala | missense |
| 19:19544100:C:T | ENST00000291495.5:c.1555C > T | ENSP00000291495.3:p.Arg519Trp | missense |
| 19:19544104:T:C | ENST00000291495.5:c.1559T > C | ENSP00000291495.3:p.Leu520Pro | missense |
| 19:19544113:CTT:C | ENST00000291495.5:c.1571_1572del | ENSP00000291495.3:p.Phe524CysfsTer6 | frameshift |
| 19:19544119:T:A | ENST00000291495.5:c.1574T > A | ENSP00000291495.3:p.Val525Glu | missense |
| 19:19544124:C:G | ENST00000291495.5:c.1579C > G | ENSP00000291495.3:p.Pro527Ala | missense |
| 19:19544124:C:T | ENST00000291495.5:c.1579C > T | ENSP00000291495.3:p.Pro527Ser | missense |
| 19:19544125:C:T | ENST00000291495.5:c.1580C > T | ENSP00000291495.3:p.Pro527Leu | missense |
| 19:19544129:C:A | ENST00000291495.5:c.1584C > A | ENSP00000291495.3:p.Ser528Arg | missense |
| 19:19544130:G:A | ENST00000291495.5:c.1585G > A | ENSP00000291495.3:p.Gly529Ser | missense |
| 19:19544131:G:A | ENST00000291495.5:c.1586G > A | ENSP00000291495.3:p.Gly529Asp | missense |
| 19:19544135:G:C | ENST00000291495.5:c.1590G > C | ENSP00000291495.3:p.Glu530Asp | missense |
| 19:19544146:C:T | ENST00000291495.5:c.1601C > T | ENSP00000291495.3:p.Ala534Val | missense |
| 19:19544148:G:C | ENST00000291495.5:c.1603G > C | ENSP00000291495.3:p.Val535Leu | missense |
| 19:19544151:C:T | ENST00000291495.5:c.1606C > T | ENSP00000291495.3:p.Arg536Trp | missense |
| 19:19544152:G:A | ENST00000291495.5:c.1607G > A | ENSP00000291495.3:p.Arg536Gln | missense |
| 19:19544152:G:C | ENST00000291495.5:c.1607G > C | ENSP00000291495.3:p.Arg536Pro | missense |
| 19:19544157:T:G | ENST00000291495.5:c.1612T > G | ENSP00000291495.3:p.Leu538Val | missense |
| 19:19544160:C:T | ENST00000291495.5:c.1615C > T | ENSP00000291495.3:p.Pro539Ser | missense |
| 19:19544166:G:T | ENST00000291495.5:c.1621G > T | ENSP00000291495.3:p.Asp541Tyr | missense |
| 19:19544170:C:T | ENST00000291495.5:c.1625C > T | ENSP00000291495.3:p.Pro542Leu | missense |
| 19:19544175:G:A | ENST00000291495.5:c.1630G > A | ENSP00000291495.3:p.Gly544Ser | missense |
| 19:19544184:G:A | ENST00000291495.5:c.1639G > A | ENSP00000291495.3:p.Val547Met | missense |
| 19:19544184:G:T | ENST00000291495.5:c.1639G > T | ENSP00000291495.3:p.Val547Leu | missense |
| 19:19544190:C:T | ENST00000291495.5:c.1645C > T | ENSP00000291495.3:p.His549Tyr | missense |
| 19:19544193:G:A | ENST00000291495.5:c.1648G > A | ENSP00000291495.3:p.Glu550Lys | missense |
| 19:19544200:A:C | ENST00000291495.5:c.1655A > C | ENSP00000291495.3:p.Lys552Thr | missense |
| 19:19544202:G:C | ENST00000291495.5:c.1657G > C | ENSP00000291495.3:p.Ala553Pro | missense |
| 19:19544203:C:T | ENST00000291495.5:c.1658C > T | ENSP00000291495.3:p.Ala553Val | missense |
| 19:19544205:A:T | ENST00000291495.5:c.1660A > T | ENSP00000291495.3:p.Met554Leu | missense |
| 19:19544206:T:C | ENST00000291495.5:c.1661T > C | ENSP00000291495.3:p.Met554Thr | missense |
| 19:19544207:G:T | ENST00000291495.5:c.1662G > T | ENSP00000291495.3:p.Met554Ile | missense |
| 19:19544208:C:T | ENST00000291495.5:c.1663C > T | ENSP00000291495.3:p.Arg555Trp | missense |
| 19:19544214:A:G | ENST00000291495.5:c.1669A > G | ENSP00000291495.3:p.Lys557Glu | missense |
| 19:19544217:G:A | ENST00000291495.5:c.1672G > A | ENSP00000291495.3:p.Ala558Thr | missense |
| 19:19544217:G:GCC | ENST00000291495.5:c.1675_1676dup | ENSP00000291495.3:p.Val560ArgfsTer49 | frameshift |
| 19:19544221:C:T | ENST00000291495.5:c.1676C > T | ENSP00000291495.3:p.Pro559Leu | missense |
| 19:19544227:T:C | ENST00000291495.5:c.1682T > C | ENSP00000291495.3:p.Ile561Thr | missense |
| 19:19544232:C:T | ENST00000291495.5:c.1687C > T | ENSP00000291495.3:p.His563Tyr | missense |
| 19:19544235:A:G | ENST00000291495.5:c.1690A > G | ENSP00000291495.3:p.Thr564Ala | missense |
| 19:19544236:C:A | ENST00000291495.5:c.1691C > A | ENSP00000291495.3:p.Thr564Asn | missense |
| 19:19544236:C:G | ENST00000291495.5:c.1691C > G | ENSP00000291495.3:p.Thr564Ser | missense |
| 19:19544236:C:T | ENST00000291495.5:c.1691C > T | ENSP00000291495.3:p.Thr564Ile | missense |
| 19:19544244:A:G | ENST00000291495.5:c.1699A > G | ENSP00000291495.3:p.Ser567Gly | missense |
| 19:19544245:G:A | ENST00000291495.5:c.1700G > A | ENSP00000291495.3:p.Ser567Asn | missense |
| 19:19544246:C:G | ENST00000291495.5:c.1701C > G | ENSP00000291495.3:p.Ser567Arg | missense |
| 19:19544249:C:G | ENST00000291495.5:c.1704C > G | ENSP00000291495.3:p.Asn568Lys | missense |
| 19:19544251:C:T | ENST00000291495.5:c.1706C > T | ENSP00000291495.3:p.Thr569Met | missense |
| 19:19544255:C:G | ENST00000291495.5:c.1710C > G | ENSP00000291495.3:p.Ile570Met | missense |
| 19:19544259:C:CT | ENST00000291495.5:c.1715dup | ENSP00000291495.3:p.Glu574ArgfsTer5 | frameshift |
| 19:19544263:G:A | ENST00000291495.5:c.1718G > A | ENSP00000291495.3:p.Gly573Asp | missense |
| 19:19544263:G:C | ENST00000291495.5:c.1718G > C | ENSP00000291495.3:p.Gly573Ala | missense |
| 19:19544265:G:C | ENST00000291495.5:c.1720G > C | ENSP00000291495.3:p.Glu574Gln | missense |
| 19:19544267:G:T | ENST00000291495.5:c.1722G > T | ENSP00000291495.3:p.Glu574Asp | missense |
| 19:19544269:T:C | ENST00000291495.5:c.1724T > C | ENSP00000291495.3:p.Leu575Pro | missense |
| 19:19544281:C:G | ENST00000291495.5:c.1736C > G | ENSP00000291495.3:p.Ala579Gly | missense |
| 19:19544281:C:T | ENST00000291495.5:c.1736C > T | ENSP00000291495.3:p.Ala579Val | missense |
| 19:19544283:C:T | ENST00000291495.5:c.1738C > T | ENSP00000291495.3:p.Pro580Ser | missense |
| 19:19544284:C:T | ENST00000291495.5:c.1739C > T | ENSP00000291495.3:p.Pro580Leu | missense |
| 19:19544286:CT:C | ENST00000291495.5:c.1742del | ENSP00000291495.3:p.Leu581ArgfsTer27 | frameshift |
| 19:19544287:T:G | ENST00000291495.5:c.1742T > G | ENSP00000291495.3:p.Leu581Arg | missense |
| 19:19544292:G:C | ENST00000291495.5:c.1747G > C | ENSP00000291495.3:p.Glu583Gln | missense |
| 19:19544299:T:G | ENST00000291495.5:c.1754T > G | ENSP00000291495.3:p.Val585Gly | missense |
| 19:19544304:C:A | ENST00000291495.5:c.1759C > A | ENSP00000291495.3:p.Pro587Thr | missense |
| 19:19544311:G:A | ENST00000291495.5:c.1766G > A | ENSP00000291495.3:p.Gly589Asp | missense |
| 19:19544313:G:A | ENST00000291495.5:c.1768G > A | ENSP00000291495.3:p.Ala590Thr | missense |
| 19:19544319:C:T | ENST00000291495.5:c.1774C > T | ENSP00000291495.3:p.Arg592Cys | missense |
| 19:19544323:G:A | ENST00000291495.5:c.1778G > A | ENSP00000291495.3:p.Arg593Lys | missense |
| 19:19544326:C:A | ENST00000291495.5:c.1781C > A | ENSP00000291495.3:p.Ala594Asp | missense |
| 19:19544328:G:A | ENST00000291495.5:c.1783G > A | ENSP00000291495.3:p.Asp595Asn | missense |
| 19:19544328:G:C | ENST00000291495.5:c.1783G > C | ENSP00000291495.3:p.Asp595His | missense |
| 19:19544328:G:T | ENST00000291495.5:c.1783G > T | ENSP00000291495.3:p.Asp595Tyr | missense |
| 19:19544330:C:G | ENST00000291495.5:c.1785C > G | ENSP00000291495.3:p.Asp595Glu | missense |
| 19:19544331:G:A | ENST00000291495.5:c.1786G > A | ENSP00000291495.3:p.Gly596Ser | missense |
| 19:19544331:G:C | ENST00000291495.5:c.1786G > C | ENSP00000291495.3:p.Gly596Arg | missense |
| 19:19544344:C:T | ENST00000291495.5:c.1799C > T | ENSP00000291495.3:p.Ser600Leu | missense |
| 19:19544350:C:T | ENST00000291495.5:c.1805C > T | ENSP00000291495.3:p.Pro602Leu | missense |
| 19:19544361:C:T | ENST00000291495.5:c.1816C > T | ENSP00000291495.3:p.Arg606Trp | missense |
| 19:19544362:G:A | ENST00000291495.5:c.1817G > A | ENSP00000291495.3:p.Arg606Gln | missense |
| 19:19544364:G:A | ENST00000291495.5:c.1819G > A | ENSP00000291495.3:p.Val607Met | missense |
| 19:19544373:G:A | ENST00000291495.5:c.1828G > A | ENSP00000291495.3:p.Val610Met | missense |
| 19:19544373:G:T | ENST00000291495.5:c.1828G > T | ENSP00000291495.3:p.Val610Leu | missense |
| 19:19544377:A:AC | ENST00000291495.5:c.1837dup | ENSP00000291495.3:p.Arg613ProfsTer12 | frameshift |
| 19:19544379:C:G | ENST00000291495.5:c.1834C > G | ENSP00000291495.3:p.Pro612Ala | missense |
| 19:19544383:G:A | ENST00000291495.5:c.1838G > A | ENSP00000291495.3:p.Arg613Gln | missense |
| 19:19544387:C:A | ENST00000291495.5:c.1842C > A | ENSP00000291495.3:p.Asp614Glu | missense |
| 19:19544392:C:G | ENST00000291495.5:c.1847C > G | ENSP00000291495.3:p.Thr616Ser | missense |
| 19:19544392:C:T | ENST00000291495.5:c.1847C > T | ENSP00000291495.3:p.Thr616Ile | missense |
| 19:19544394:T:G | ENST00000291495.5:c.1849T > G | ENSP00000291495.3:p.Ser617Ala | missense |
| 19:19544395:C:T | ENST00000291495.5:c.1850C > T | ENSP00000291495.3:p.Ser617Leu | missense |
| 19:19544395:CG:C | ENST00000291495.5:c.1852del | ENSP00000291495.3:p.Ala618ArgfsTer67 | frameshift |
| 19:19544397:G:A | ENST00000291495.5:c.1852G > A | ENSP00000291495.3:p.Ala618Thr | missense |
| 19:19544400:G:A | ENST00000291495.5:c.1855G > A | ENSP00000291495.3:p.Ala619Thr | missense |
| 19:19544406:G:GC | ENST00000291495.5:c.1866dup | ENSP00000291495.3:p.Ser623GlnfsTer2 | frameshift |
| 19:19544407:C:T | ENST00000291495.5:c.1862C > T | ENSP00000291495.3:p.Ala621Val | missense |
| 19:19544421:C:A | ENST00000291495.5:c.1876C > A | ENSP00000291495.3:p.Arg626Ser | missense |
| 19:19544421:C:T | ENST00000291495.5:c.1876C > T | ENSP00000291495.3:p.Arg626Cys | missense |
| 19:19544422:G:A | ENST00000291495.5:c.1877G > A | ENSP00000291495.3:p.Arg626His | missense |
| 19:19544431:A:T | ENST00000291495.5:c.1886A > T | ENSP00000291495.3:p.Asp629Val | missense |
| 19:19544434:G:A | ENST00000291495.5:c.1889G > A | ENSP00000291495.3:p.Ser630Asn | missense |
| 19:19544436:G:T | ENST00000291495.5:c.1891G > T | ENSP00000291495.3:p.Asp631Tyr | missense |
| 19:19544438:C:A | ENST00000291495.5:c.1893C > A | ENSP00000291495.3:p.Asp631Glu | missense |
| 19:19544439:G:A | ENST00000291495.5:c.1894G > A | ENSP00000291495.3:p.Gly632Ser | missense |
| 19:19544439:G:C | ENST00000291495.5:c.1894G > C | ENSP00000291495.3:p.Gly632Arg | missense |
| 19:19544442:G:A | ENST00000291495.5:c.1897G > A | ENSP00000291495.3:p.Glu633Lys | missense |
| 19:19544442:G:C | ENST00000291495.5:c.1897G > C | ENSP00000291495.3:p.Glu633Gln | missense |
| 19:19544443:A:G | ENST00000291495.5:c.1898A > G | ENSP00000291495.3:p.Glu633Gly | missense |
| 19:19544451:C:T | ENST00000291495.5:c.1906C > T | ENSP00000291495.3:p.Pro636Ser | missense |
| 19:19544457:C:A | ENST00000291495.5:c.1912C > A | ENSP00000291495.3:p.Arg638Ser | missense |
| 19:19544458:G:A | ENST00000291495.5:c.1913G > A | ENSP00000291495.3:p.Arg638His | missense |
| 19:19544458:G:T | ENST00000291495.5:c.1913G > T | ENSP00000291495.3:p.Arg638Leu | missense |
| 19:19544461:C:G | ENST00000291495.5:c.1916C > G | ENSP00000291495.3:p.Thr639Ser | missense |
| 19:19544469:A:G | ENST00000291495.5:c.1924A > G | ENSP00000291495.3:p.Met642Val | missense |
| 19:19544470:T:C | ENST00000291495.5:c.1925T > C | ENSP00000291495.3:p.Met642Thr | missense |
| 19:19544475:TC:T | ENST00000291495.5:c.1932del | ENSP00000291495.3:p.Val645TrpfsTer40 | frameshift |
| 19:19544484:C:G | ENST00000291495.5:c.1939C > G | ENSP00000291495.3:p.Leu647Val | missense |
| 19:19544484:C:T | ENST00000291495.5:c.1939C > T | ENSP00000291495.3:p.Leu647Phe | missense |
| 19:19544487:C:T | ENST00000291495.5:c.1942C > T | ENSP00000291495.3:p.Arg648Cys | missense |
| 19:19544488:G:A | ENST00000291495.5:c.1943G > A | ENSP00000291495.3:p.Arg648His | missense |
| 19:19544488:G:T | ENST00000291495.5:c.1943G > T | ENSP00000291495.3:p.Arg648Leu | missense |
| 19:19544490:G:C | ENST00000291495.5:c.1945G > C | ENSP00000291495.3:p.Ala649Pro | missense |
| 19:19544490:G:T | ENST00000291495.5:c.1945G > T | ENSP00000291495.3:p.Ala649Ser | missense |
| 19:19544490:GC:G | ENST00000291495.5:c.1946del | ENSP00000291495.3:p.Ala649GlyfsTer36 | frameshift |
| 19:19544491:C:A | ENST00000291495.5:c.1946C > A | ENSP00000291495.3:p.Ala649Glu | missense |
| 19:19544491:C:T | ENST00000291495.5:c.1946C > T | ENSP00000291495.3:p.Ala649Val | missense |
| 19:19544493:C:T | ENST00000291495.5:c.1948C > T | ENSP00000291495.3:p.Pro650Ser | missense |
| 19:19544496:G:A | ENST00000291495.5:c.1951G > A | ENSP00000291495.3:p.Gly651Ser | missense |
| 19:19544496:G:T | ENST00000291495.5:c.1951G > T | ENSP00000291495.3:p.Gly651Cys | missense |
| 19:19544497:G:A | ENST00000291495.5:c.1952G > A | ENSP00000291495.3:p.Gly651Asp | missense |
| 19:19544502:G:A | ENST00000291495.5:c.1957G > A | ENSP00000291495.3:p.Ala653Thr | missense |
| 19:19544502:G:C | ENST00000291495.5:c.1957G > C | ENSP00000291495.3:p.Ala653Pro | missense |
| 19:19544503:C:T | ENST00000291495.5:c.1958C > T | ENSP00000291495.3:p.Ala653Val | missense |
| 19:19544509:A:T | ENST00000291495.5:c.1964A > T | ENSP00000291495.3:p.Gln655Leu | missense |
| 19:19544510:G:C | ENST00000291495.5:c.1965G > C | ENSP00000291495.3:p.Gln655His | missense |
| 19:19544511:C:A | ENST00000291495.5:c.1966C > A | ENSP00000291495.3:p.Leu656Met | missense |
| 19:19544512:T:C | ENST00000291495.5:c.1967T > C | ENSP00000291495.3:p.Leu656Pro | missense |
| 19:19544517:G:A | ENST00000291495.5:c.1972G > A | ENSP00000291495.3:p.Val658Met | missense |
| 19:19544517:G:T | ENST00000291495.5:c.1972G > T | ENSP00000291495.3:p.Val658Leu | missense |
| 19:19544520:G:T | ENST00000291495.5:c.1975G > T | ENSP00000291495.3:p.Gly659Trp | missense |
| 19:19544521:G:A | ENST00000291495.5:c.1976G > A | ENSP00000291495.3:p.Gly659Glu | missense |
| 19:19544524:C:T | ENST00000291495.5:c.1979C > T | ENSP00000291495.3:p.Pro660Leu | missense |
| 19:19544532:G:A | ENST00000291495.5:c.1987G > A | ENSP00000291495.3:p.Val663Met | missense |
| 19:19544533:T:G | ENST00000291495.5:c.1988T > G | ENSP00000291495.3:p.Val663Gly | missense |
| 19:19544536:G:A | ENST00000291495.5:c.1991G > A | ENSP00000291495.3:p.Arg664Gln | missense |
| 19:19544548:G:C | ENST00000291495.5:c.2003G > C | ENSP00000291495.3:p.Ser668Thr | missense |
| 19:19544553:A:C | ENST00000291495.5:c.2008A > C | ENSP00000291495.3:p.Ile670Leu | missense |
| 19:19544553:A:G | ENST00000291495.5:c.2008A > G | ENSP00000291495.3:p.Ile670Val | missense |
| 19:19544558:C:A | ENST00000291495.5:c.2013C > A | ENSP00000291495.3:p.His671Gln | missense |
| 19:19544559:A:G | ENST00000291495.5:c.2014A > G | ENSP00000291495.3:p.Met672Val | missense |
| 19:19544562:C:T | ENST00000291495.5:c.2017C > T | ENSP00000291495.3:p.Pro673Ser | missense |
| 19:19544568:C:A | ENST00000291495.5:c.2023C > A | ENSP00000291495.3:p.His675Asn | missense |
| 19:19544571:G:A | ENST00000291495.5:c.2026G > A | ENSP00000291495.3:p.Val676Met | missense |
| 19:19544577:G:A | ENST00000291495.5:c.2032G > A | ENSP00000291495.3:p.Ala678Thr | missense |
| 19:19544577:G:T | ENST00000291495.5:c.2032G > T | ENSP00000291495.3:p.Ala678Ser | missense |
| 19:19544578:C:A | ENST00000291495.5:c.2033C > A | ENSP00000291495.3:p.Ala678Asp | missense |
| 19:19544578:C:T | ENST00000291495.5:c.2033C > T | ENSP00000291495.3:p.Ala678Val | missense |
| 19:19544580:C:A | ENST00000291495.5:c.2035C > A | ENSP00000291495.3:p.Leu679Ile | missense |
| 19:19544580:C:T | ENST00000291495.5:c.2035C > T | ENSP00000291495.3:p.Leu679Phe | missense |
| 19:19544593:C:G | ENST00000291495.5:c.2048C > G | ENSP00000291495.3:p.Ser683Trp | missense |
| 19:19544593:C:T | ENST00000291495.5:c.2048C > T | ENSP00000291495.3:p.Ser683Leu | missense |
| 19:19544598:A:C | ENST00000291495.5:c.2053A > C | ENSP00000291495.3:p.Asn685His | missense |
| 19:19544602:C:G | ENST00000291495.5:c.2057C > G | ENSP00000291495.3:p.Pro686Arg | missense |
| 19:19544605:A:C | ENST00000291495.5:c.2060A > C | ENSP00000291495.3:p.Glu687Ala | missense |
| 19:19544606:G:T | ENST00000291495.5:c.2061G > T | ENSP00000291495.3:p.Glu687Asp | missense |
| 19:19544608:C:A | ENST00000291495.5:c.2063C > A | ENSP00000291495.3:p.Thr688Asn | missense |
| 19:19544610:G:A | ENST00000291495.5:c.2065G > A | ENSP00000291495.3:p.Gly689Ser | missense |
| 19:19544622:G:A | ENST00000291495.5:c.2077G > A | ENSP00000291495.3:p.Glu693Lys | missense |
| 19:19544625:G:T | ENST00000291495.5:c.2080G > T | ENSP00000291495.3:p.Glu694Ter | stop_gained |
| 19:19544632:G:A | ENST00000291495.5:c.2087G > A | ENSP00000291495.3:p.Gly696Asp | missense |
| 19:19544637:C:T | ENST00000291495.5:c.2092C > T | ENSP00000291495.3:p.Arg698Trp | missense |
| 19:19544638:G:A | ENST00000291495.5:c.2093G > A | ENSP00000291495.3:p.Arg698Gln | missense |
| 19:19544640:C:A | ENST00000291495.5:c.2095C > A | ENSP00000291495.3:p.Arg699Ser | missense |
| 19:19544640:C:T | ENST00000291495.5:c.2095C > T | ENSP00000291495.3:p.Arg699Cys | missense |
| 19:19544641:G:A | ENST00000291495.5:c.2096G > A | ENSP00000291495.3:p.Arg699His | missense |
| 19:19544643:G:A | ENST00000291495.5:c.2098G > A | ENSP00000291495.3:p.Glu700Lys | missense |
| 19:19544645:G:GGGGTCCTC | ENST00000291495.5:c.2104_2111dup | ENSP00000291495.3:p.Val707AlafsTer36 | frameshift |
| 19:19544646:G:T | ENST00000291495.5:c.2101G > T | ENSP00000291495.3:p.Gly701Trp | missense |
| 19:19544653:C:T | ENST00000291495.5:c.2108C > T | ENSP00000291495.3:p.Ser703Leu | missense |
| 19:19544656:G:A | ENST00000291495.5:c.2111G > A | ENSP00000291495.3:p.Gly704Asp | missense |
| 19:19544658:C:T | ENST00000291495.5:c.2113C > T | ENSP00000291495.3:p.Pro705Ser | missense |
| 19:19544661:C:T | ENST00000291495.5:c.2116C > T | ENSP00000291495.3:p.Arg706Trp | missense |
| 19:19544662:G:A | ENST00000291495.5:c.2117G > A | ENSP00000291495.3:p.Arg706Gln | missense |
| 19:19544671:G:A | ENST00000291495.5:c.2126G > A | ENSP00000291495.3:p.Arg709Gln | missense |
| 19:19544671:G:T | ENST00000291495.5:c.2126G > T | ENSP00000291495.3:p.Arg709Leu | missense |
| 19:19544673:G:C | ENST00000291495.5:c.2128G > C | ENSP00000291495.3:p.Glu710Gln | missense |
| 19:19544678:G:T | ENST00000291495.5:c.2133G > T | ENSP00000291495.3:p.Glu711Asp | missense |
| 19:19544679:C:A | ENST00000291495.5:c.2134C > A | ENSP00000291495.3:p.Arg712Ser | missense |
| 19:19544680:G:A | ENST00000291495.5:c.2135G > A | ENSP00000291495.3:p.Arg712His | missense |
| 19:19544680:G:T | ENST00000291495.5:c.2135G > T | ENSP00000291495.3:p.Arg712Leu | missense |
| 19:19544682:G:A | ENST00000291495.5:c.2137G > A | ENSP00000291495.3:p.Val713Ile | missense |
| 19:19544689:T:C | ENST00000291495.5:c.2144T > C | ENSP00000291495.3:p.Leu715Pro | missense |
| 19:19544691:G:A | ENST00000291495.5:c.2146G > A | ENSP00000291495.3:p.Val716Met | missense |
| 19:19544691:GT:G | ENST00000291495.5:c.2147del | ENSP00000291495.3:p.Val716GlyfsTer24 | frameshift |
| 19:19544699:C:G | ENST00000291495.5:c.2154C > G | ENSP00000291495.3:p.Asn718Lys | missense |
| 19:19544700:G:A | ENST00000291495.5:c.2155G > A | ENSP00000291495.3:p.Val719Met | missense |
| 19:19544707:TC:T | ENST00000291495.5:c.2164del | ENSP00000291495.3:p.Arg722GlyfsTer18 | frameshift |
| 19:19544709:C:T | ENST00000291495.5:c.2164C > T | ENSP00000291495.3:p.Arg722Trp | missense |
| 19:19544712:G:A | ENST00000291495.5:c.2167G > A | ENSP00000291495.3:p.Glu723Lys | missense |
| 19:19544712:G:C | ENST00000291495.5:c.2167G > C | ENSP00000291495.3:p.Glu723Gln | missense |
| 19:19544714:G:C | ENST00000291495.5:c.2169G > C | ENSP00000291495.3:p.Glu723Asp | missense |
| 19:19544718:C:G | ENST00000291495.5:c.2173C > G | ENSP00000291495.3:p.Arg725Gly | missense |
| 19:19544719:G:A | ENST00000291495.5:c.2174G > A | ENSP00000291495.3:p.Arg725His | missense |
| 19:19544725:T:A | ENST00000291495.5:c.2180T > A | ENSP00000291495.3:p.Phe727Tyr | missense |
| 19:19544731:T:C | ENST00000291495.5:c.2186T > C | ENSP00000291495.3:p.Leu729Pro | missense |
| 19:19544735:C:A | ENST00000291495.5:c.2190C > A | ENSP00000291495.3:p.Asp730Glu | missense |
| 19:19544737:T:C | ENST00000291495.5:c.2192T > C | ENSP00000291495.3:p.Val731Ala | missense |
| 19:19544739:C:A | ENST00000291495.5:c.2194C > A | ENSP00000291495.3:p.Pro732Thr | missense |
| 19:19544739:C:T | ENST00000291495.5:c.2194C > T | ENSP00000291495.3:p.Pro732Ser | missense |
| 19:19544740:C:T | ENST00000291495.5:c.2195C > T | ENSP00000291495.3:p.Pro732Leu | missense |
| 19:19544743:A:C | ENST00000291495.5:c.2198A > C | ENSP00000291495.3:p.Glu733Ala | missense |
| 19:19544743:A:G | ENST00000291495.5:c.2198A > G | ENSP00000291495.3:p.Glu733Gly | missense |
| 19:19544748:C:T | ENST00000291495.5:c.2203C > T | ENSP00000291495.3:p.Arg735Cys | missense |
| 19:19544752:G:A | ENST00000291495.5:c.2207G > A | ENSP00000291495.3:p.Arg736His | missense |
| 19:19544752:G:C | ENST00000291495.5:c.2207G > C | ENSP00000291495.3:p.Arg736Pro | missense |
| 19:19544754:T:G | ENST00000291495.5:c.2209T > G | ENSP00000291495.3:p.Cys737Gly | missense |
| 19:19544759:C:A | ENST00000291495.5:c.2214C > A | ENSP00000291495.3:p.Phe738Leu | missense |
| 19:19544759:C:G | ENST00000291495.5:c.2214C > G | ENSP00000291495.3:p.Phe738Leu | missense |
| 19:19544760:G:A | ENST00000291495.5:c.2215G > A | ENSP00000291495.3:p.Val739Met | missense |
| 19:19544760:G:T | ENST00000291495.5:c.2215G > T | ENSP00000291495.3:p.Val739Leu | missense |
| 19:19544763:A:T | ENST00000291495.5:c.2218A > T | ENSP00000291495.3:p.Lys740Ter | stop_gained |
| 19:19544766:G:C | ENST00000291495.5:c.2221G > C | ENSP00000291495.3:p.Val741Leu | missense |
| 19:19544767:T:C | ENST00000291495.5:c.2222T > C | ENSP00000291495.3:p.Val741Ala | missense |
| 19:19544769:C:A | ENST00000291495.5:c.2224C > A | ENSP00000291495.3:p.Arg742Ser | missense |
| 19:19544769:C:T | ENST00000291495.5:c.2224C > T | ENSP00000291495.3:p.Arg742Cys | missense |
| 19:19544770:G:A | ENST00000291495.5:c.2225G > A | ENSP00000291495.3:p.Arg742His | missense |
| 19:19544772:G:A | ENST00000291495.5:c.2227G > A | ENSP00000291495.3:p.Ala743Thr | missense |
| 19:19544775:T:C | ENST00000291495.5:c.2230T > C | ENSP00000291495.3:p.Tyr744His | missense |
| 19:19544777:C:G | ENST00000291495.5:c.2232C > G | ENSP00000291495.3:p.Tyr744Ter | stop_gained |
| 19:19544778:G:A | ENST00000291495.5:c.2233G > A | ENSP00000291495.3:p.Ala745Thr | missense |
| 19:19544778:G:C | ENST00000291495.5:c.2233G > C | ENSP00000291495.3:p.Ala745Pro | missense |
| 19:19544783:C:A | ENST00000291495.5:c.2238C > A | ENSP00000291495.3:p.Asn746Lys | missense |
| 19:19544788:A:G | ENST00000291495.5:c.2243A > G | ENSP00000291495.3:p.Lys748Arg | missense |
| 19:19544789:G:T | ENST00000291495.5:c.2244G > T | ENSP00000291495.3:p.Lys748Asn | missense |
| 19:19544800:G:A | ENST00000291495.5:c.2255G > A | ENSP00000291495.3:p.Ser752Asn | missense |
| 19:19544801:C:A | ENST00000291495.5:c.2256C > A | ENSP00000291495.3:p.Ser752Arg | missense |
| 19:19544808:G:A | ENST00000291495.5:c.2263G > A | ENSP00000291495.3:p.Val755Met | missense |
| 19:19544808:G:C | ENST00000291495.5:c.2263G > C | ENSP00000291495.3:p.Val755Leu | missense |
| 19:19544814:G:A | ENST00000291495.5:c.2269G > A | ENSP00000291495.3:p.Gly757Ser | missense |
| 19:19544815:G:C | ENST00000291495.5:c.2270G > C | ENSP00000291495.3:p.Gly757Ala | missense |
| 19:19544823:G:A | ENST00000291495.5:c.2278G > A | ENSP00000291495.3:p.Val760Ile | missense |
| 19:19544827:C:T | ENST00000291495.5:c.2282C > T | ENSP00000291495.3:p.Thr761Met | missense |
| 19:19544832:G:C | ENST00000291495.5:c.2287G > C | ENSP00000291495.3:p.Val763Leu | missense |
| 19:19544844:C:A | ENST00000291495.5:c.2299C > A | ENSP00000291495.3:p.Pro767Thr | missense |
| 19:19544845:C:A | ENST00000291495.5:c.2300C > A | ENSP00000291495.3:p.Pro767His | missense |
| 19:19544847:G:A | ENST00000291495.5:c.2302G > A | ENSP00000291495.3:p.Ala768Thr | missense |
| 19:19544848:C:G | ENST00000291495.5:c.2303C > G | ENSP00000291495.3:p.Ala768Gly | missense |
| 19:19544848:C:T | ENST00000291495.5:c.2303C > T | ENSP00000291495.3:p.Ala768Val | missense |
| 19:19544853:G:C | ENST00000291495.5:c.2308G > C | ENSP00000291495.3:p.Gly770Arg | missense |
| 19:19544856:T:G | ENST00000291495.5:c.2311T > G | ENSP00000291495.3:p.Phe771Val | missense |
| 19:19544862:G:A | ENST00000291495.5:c.2317G > A | ENSP00000291495.3:p.Ala773Thr | missense |
| 19:19544866:A:AC | ENST00000291495.5:c.2326dup | ENSP00000291495.3:p.Arg776ProfsTer7 | frameshift |
| 19:19544868:C:T | ENST00000291495.5:c.2323C > T | ENSP00000291495.3:p.Pro775Ser | missense |
| 19:19544871:C:G | ENST00000291495.5:c.2326C > G | ENSP00000291495.3:p.Arg776Gly | missense |
| 19:19544871:C:T | ENST00000291495.5:c.2326C > T | ENSP00000291495.3:p.Arg776Cys | missense |
| 19:19544872:G:A | ENST00000291495.5:c.2327G > A | ENSP00000291495.3:p.Arg776His | missense |
| 19:19544872:G:T | ENST00000291495.5:c.2327G > T | ENSP00000291495.3:p.Arg776Leu | missense |
| 19:19544875:C:T | ENST00000291495.5:c.2330C > T | ENSP00000291495.3:p.Ala777Val | missense |
| 19:19544883:C:G | ENST00000291495.5:c.2338C > G | ENSP00000291495.3:p.Arg780Gly | missense |
| 19:19544883:C:T | ENST00000291495.5:c.2338C > T | ENSP00000291495.3:p.Arg780Cys | missense |
| 19:19544884:G:A | ENST00000291495.5:c.2339G > A | ENSP00000291495.3:p.Arg780His | missense |
| 19:19544884:G:C | ENST00000291495.5:c.2339G > C | ENSP00000291495.3:p.Arg780Pro | missense |
| 19:19544889:G:C | ENST00000291495.5:c.2344G > C | ENSP00000291495.3:p.Asp782His | missense |
| 19:19544893:G:A | ENST00000291495.5:c.2348G > A | ENSP00000291495.3:p.Ser783Asn | missense |
| 19:19544894:C:A | ENST00000291495.5:c.2349C > A | ENSP00000291495.3:p.Ser783Arg | missense |
| 19:19544895:G:A | ENST00000291495.5:c.2350G > A | ENSP00000291495.3:p.Ala784Thr | missense |
| 19:19544896:C:G | ENST00000291495.5:c.2351C > G | ENSP00000291495.3:p.Ala784Gly | missense |
| 19:19544896:C:T | ENST00000291495.5:c.2351C > T | ENSP00000291495.3:p.Ala784Val | missense |
| 19:19544902:C:T | ENST00000291495.5:c.2357C > T | ENSP00000291495.3:p.Thr786Ile | missense |
| 19:19544904:G:A | ENST00000291495.5:c.2359G > A | ENSP00000291495.3:p.Gly787Ser | missense |
| 19:19544904:G:C | ENST00000291495.5:c.2359G > C | ENSP00000291495.3:p.Gly787Arg | missense |
| 19:19544905:G:A | ENST00000291495.5:c.2360G > A | ENSP00000291495.3:p.Gly787Asp | missense |
| 19:19544905:G:C | ENST00000291495.5:c.2360G > C | ENSP00000291495.3:p.Gly787Ala | missense |
| 19:19544911:A:G | ENST00000291495.5:c.2366A > G | ENSP00000291495.3:p.Asn789Ser | missense |
| 19:19544913:G:T | ENST00000291495.5:c.2368G > T | ENSP00000291495.3:p.Gly790Cys | missense |
| 19:19544916:G:A | ENST00000291495.5:c.2371G > A | ENSP00000291495.3:p.Ala791Thr | missense |
| 19:19544925:C:T | ENST00000291495.5:c.2380C > T | ENSP00000291495.3:p.Pro794Ser | missense |
| 19:19544928:G:A | ENST00000291495.5:c.2383G > A | ENSP00000291495.3:p.Ala795Thr | missense |
| 19:19544928:G:T | ENST00000291495.5:c.2383G > T | ENSP00000291495.3:p.Ala795Ser | missense |
| 19:19544929:C:T | ENST00000291495.5:c.2384C > T | ENSP00000291495.3:p.Ala795Val | missense |
| 19:19544935:G:C | ENST00000291495.5:c.2390G > C | ENSP00000291495.3:p.Cys797Ser | missense |
| 19:19544937:G:A | ENST00000291495.5:c.2392G > A | ENSP00000291495.3:p.Asp798Asn | missense |
| 19:19544937:G:T | ENST00000291495.5:c.2392G > T | ENSP00000291495.3:p.Asp798Tyr | missense |
| 19:19544940:G:A | ENST00000291495.5:c.2395G > A | ENSP00000291495.3:p.Ala799Thr | missense |
| 19:19544940:G:C | ENST00000291495.5:c.2395G > C | ENSP00000291495.3:p.Ala799Pro | missense |
| 19:19544943:G:A | ENST00000291495.5:c.2398G > A | ENSP00000291495.3:p.Asp800Asn | missense |
| 19:19544945:C:A | ENST00000291495.5:c.2400C > A | ENSP00000291495.3:p.Asp800Glu | missense |
| 19:19544945:C:G | ENST00000291495.5:c.2400C > G | ENSP00000291495.3:p.Asp800Glu | missense |
| 19:19544947:G:A | ENST00000291495.5:c.2402G > A | ENSP00000291495.3:p.Arg801Lys | missense |
| 19:19544948:G:C | ENST00000291495.5:c.2403G > C | ENSP00000291495.3:p.Arg801Ser | missense |
| 19:19544954:C:G | ENST00000291495.5:c.2409C > G | ENSP00000291495.3:p.Asp803Glu | missense |
| 19:19544961:A:G | ENST00000291495.5:c.2416A > G | ENSP00000291495.3:p.Thr806Ala | missense |
| 19:19544962:C:T | ENST00000291495.5:c.2417C > T | ENSP00000291495.3:p.Thr806Ile | missense |
| 19:19544970:G:C | ENST00000291495.5:c.2425G > C | ENSP00000291495.3:p.Val809Leu | missense |
| 19:19544971:T:C | ENST00000291495.5:c.2426T > C | ENSP00000291495.3:p.Val809Ala | missense |
| 19:19544976:G:A | ENST00000291495.5:c.2431G > A | ENSP00000291495.3:p.Ala811Thr | missense |
| 19:19544980:C:A | ENST00000291495.5:c.2435C > A | ENSP00000291495.3:p.Thr812Asn | missense |
| 19:19544982:C:G | ENST00000291495.5:c.2437C > G | ENSP00000291495.3:p.Leu813Val | missense |
| 19:19544983:T:C | ENST00000291495.5:c.2438T > C | ENSP00000291495.3:p.Leu813Pro | missense |
| 19:19544985:G:T | ENST00000291495.5:c.2440G > T | ENSP00000291495.3:p.Gly814Cys | missense |
| 19:19544992:A:C | ENST00000291495.5:c.2447A > C | ENSP00000291495.3:p.Glu816Ala | missense |
| 19:19544995:A:G | ENST00000291495.5:c.2450A > G | ENSP00000291495.3:p.Glu817Gly | missense |
| 19:19544997:C:G | ENST00000291495.5:c.2452C > G | ENSP00000291495.3:p.Leu818Val | missense |
| 19:19545000:G:A | ENST00000291495.5:c.2455G > A | ENSP00000291495.3:p.Glu819Lys | missense |
| 19:19545000:G:C | ENST00000291495.5:c.2455G > C | ENSP00000291495.3:p.Glu819Gln | missense |
| 19:19545006:G:A | ENST00000291495.5:c.2461G > A | ENSP00000291495.3:p.Ala821Thr | missense |
| 19:19545007:C:G | ENST00000291495.5:c.2462C > G | ENSP00000291495.3:p.Ala821Gly | missense |
| 19:19545010:C:T | ENST00000291495.5:c.2465C > T | ENSP00000291495.3:p.Pro822Leu | missense |
| 19:19545022:G:A | ENST00000291495.5:c.2477G > A | ENSP00000291495.3:p.Arg826His | missense |
| 19:19545024:C:T | ENST00000291495.5:c.2479C > T | ENSP00000291495.3:p.Pro827Ser | missense |
| 19:19545030:C:G | ENST00000291495.5:c.2485C > G | ENSP00000291495.3:p.Pro829Ala | missense |
| 19:19545031:C:T | ENST00000291495.5:c.2486C > T | ENSP00000291495.3:p.Pro829Leu | missense |
| 19:19545039:G:A | ENST00000291495.5:c.2494G > A | ENSP00000291495.3:p.Val832Met | missense |
| 19:19545039:G:T | ENST00000291495.5:c.2494G > T | ENSP00000291495.3:p.Val832Leu | missense |
| 19:19545045:G:A | ENST00000291495.5:c.2500G > A | ENSP00000291495.3:p.Val834Ile | missense |
| 19:19545055:C:T | ENST00000291495.5:c.2510C > T | ENSP00000291495.3:p.Pro837Leu | missense |
| 19:19545058:A:G | ENST00000291495.5:c.2513A > G | ENSP00000291495.3:p.Tyr838Cys | missense |
| 19:19545058:AC:A | ENST00000291495.5:c.2515del | ENSP00000291495.3:p.Leu839TrpfsTer54 | frameshift |
| 19:19545060:C:A | ENST00000291495.5:c.2515C > A | ENSP00000291495.3:p.Leu839Met | missense |
| 19:19545066:A:G | ENST00000291495.5:c.2521A > G | ENSP00000291495.3:p.Arg841Gly | missense |
| 19:19545068:G:C | ENST00000291495.5:c.2523G > C | ENSP00000291495.3:p.Arg841Ser | missense |
| 19:19545070:T:G | ENST00000291495.5:c.2525T > G | ENSP00000291495.3:p.Leu842Arg | missense |
| 19:19545072:G:T | ENST00000291495.5:c.2527G > T | ENSP00000291495.3:p.Gly843Trp | missense |
| 19:19545073:G:A | ENST00000291495.5:c.2528G > A | ENSP00000291495.3:p.Gly843Glu | missense |
| 19:19545073:G:C | ENST00000291495.5:c.2528G > C | ENSP00000291495.3:p.Gly843Ala | missense |
| 19:19545076:A:G | ENST00000291495.5:c.2531A > G | ENSP00000291495.3:p.Tyr844Cys | missense |
| 19:19545079:G:A | ENST00000291495.5:c.2534G > A | ENSP00000291495.3:p.Arg845His | missense |
| 19:19545079:G:C | ENST00000291495.5:c.2534G > C | ENSP00000291495.3:p.Arg845Pro | missense |
| 19:19545081:C:G | ENST00000291495.5:c.2536C > G | ENSP00000291495.3:p.Arg846Gly | missense |
| 19:19545081:C:T | ENST00000291495.5:c.2536C > T | ENSP00000291495.3:p.Arg846Trp | missense |
| 19:19545082:G:A | ENST00000291495.5:c.2537G > A | ENSP00000291495.3:p.Arg846Gln | missense |
| 19:19545082:G:T | ENST00000291495.5:c.2537G > T | ENSP00000291495.3:p.Arg846Leu | missense |
| 19:19545085:C:G | ENST00000291495.5:c.2540C > G | ENSP00000291495.3:p.Thr847Arg | missense |
| 19:19545087:G:C | ENST00000291495.5:c.2542G > C | ENSP00000291495.3:p.Asp848His | missense |
| 19:19545090:C:G | ENST00000291495.5:c.2545C > G | ENSP00000291495.3:p.His849Asp | missense |
| 19:19545091:ACGACGATC:A | ENST00000291495.5:c.2548_2555del | ENSP00000291495.3:p.Asp850ArgfsTer5 | frameshift |
| 19:19545092:C:G | ENST00000291495.5:c.2547C > G | ENSP00000291495.3:p.His849Gln | missense |
| 19:19545093:G:T | ENST00000291495.5:c.2548G > T | ENSP00000291495.3:p.Asp850Tyr | missense |
| 19:19545094:A:G | ENST00000291495.5:c.2549A > G | ENSP00000291495.3:p.Asp850Gly | missense |
| 19:19545095:C:A | ENST00000291495.5:c.2550C > A | ENSP00000291495.3:p.Asp850Glu | missense |
| 19:19545095:C:G | ENST00000291495.5:c.2550C > G | ENSP00000291495.3:p.Asp850Glu | missense |
| 19:19545097:A:G | ENST00000291495.5:c.2552A > G | ENSP00000291495.3:p.Asp851Gly | missense |
| 19:19545098:T:A | ENST00000291495.5:c.2553T > A | ENSP00000291495.3:p.Asp851Glu | missense |
| 19:19545111:C:G | ENST00000291495.5:c.2566C > G | ENSP00000291495.3:p.Arg856Gly | missense |
| 19:19545112:G:A | ENST00000291495.5:c.2567G > A | ENSP00000291495.3:p.Arg856His | missense |
| 19:19545112:G:C | ENST00000291495.5:c.2567G > C | ENSP00000291495.3:p.Arg856Pro | missense |
| 19:19545123:C:G | ENST00000291495.5:c.2578C > G | ENSP00000291495.3:p.Arg860Gly | missense |
| 19:19545124:G:A | ENST00000291495.5:c.2579G > A | ENSP00000291495.3:p.Arg860His | missense |
| 19:19545124:G:GC | ENST00000291495.5:c.2580dup | ENSP00000291495.3:p.Ile861HisfsTer10 | frameshift |
| 19:19545124:G:T | ENST00000291495.5:c.2579G > T | ENSP00000291495.3:p.Arg860Leu | missense |
| 19:19545131:C:A | ENST00000291495.5:c.2586C > A | ENSP00000291495.3:p.Asn862Lys | missense |
| 19:19545133:T:A | ENST00000291495.5:c.2588T > A | ENSP00000291495.3:p.Leu863His | missense |
| 19:19545135:G:C | ENST00000291495.5:c.2590G > C | ENSP00000291495.3:p.Ala864Pro | missense |
| 19:19545141:C:T | ENST00000291495.5:c.2596C > T | ENSP00000291495.3:p.Pro866Ser | missense |
| 19:19545147:C:G | ENST00000291495.5:c.2602C > G | ENSP00000291495.3:p.Pro868Ala | missense |
| 19:19545151:G:A | ENST00000291495.5:c.2606G > A | ENSP00000291495.3:p.Gly869Asp | missense |
| 19:19545154:A:AC | ENST00000291495.5:c.2613dup | ENSP00000291495.3:p.Ala872ArgfsTer195 | frameshift |
| 19:19545154:A:C | ENST00000291495.5:c.2609A > C | ENSP00000291495.3:p.Asp870Ala | missense |
| 19:19545160:C:T | ENST00000291495.5:c.2615C > T | ENSP00000291495.3:p.Ala872Val | missense |
| 19:19545162:G:A | ENST00000291495.5:c.2617G > A | ENSP00000291495.3:p.Glu873Lys | missense |
| 19:19545165:G:A | ENST00000291495.5:c.2620G > A | ENSP00000291495.3:p.Ala874Thr | missense |
| 19:19545166:C:T | ENST00000291495.5:c.2621C > T | ENSP00000291495.3:p.Ala874Val | missense |
| 19:19545173:GC:G | ENST00000291495.5:c.2630del | ENSP00000291495.3:p.Pro877LeufsTer16 | frameshift |
| 19:19545175:C:T | ENST00000291495.5:c.2630C > T | ENSP00000291495.3:p.Pro877Leu | missense |
| 19:19545184:C:G | ENST00000291495.5:c.2639C > G | ENSP00000291495.3:p.Pro880Arg | missense |
| 19:19545184:C:T | ENST00000291495.5:c.2639C > T | ENSP00000291495.3:p.Pro880Leu | missense |
| 19:19545188:G:C | ENST00000291495.5:c.2643G > C | ENSP00000291495.3:p.Trp881Cys | missense |
| 19:19545190:G:A | ENST00000291495.5:c.2645G > A | ENSP00000291495.3:p.Arg882His | missense |
| 19:19545194:C:A | ENST00000291495.5:c.2649C > A | ENSP00000291495.3:p.Ser883Arg | missense |
| 19:19545196:TGC:T | ENST00000291495.5:c.2653_2654del | ENSP00000291495.3:p.Arg885GlyfsTer181 | frameshift |
| 19:19545198:C:T | ENST00000291495.5:c.2653C > T | ENSP00000291495.3:p.Arg885Trp | missense |
| 19:19545202:A:T | ENST00000291495.5:c.2657A > T | ENSP00000291495.3:p.Glu886Val | missense |
| 19:19545203:A:ACCCG | ENST00000291495.5:c.2658_2659insCCCG | ENSP00000291495.3:p.Cys887ProfsTer181 | frameshift |
| 19:19545205:G:C | ENST00000291495.5:c.2660G > C | ENSP00000291495.3:p.Cys887Ser | missense |
| 19:19545206:C:G | ENST00000291495.5:c.2661C > G | ENSP00000291495.3:p.Cys887Trp | missense |
| 19:19545208:A:ACCGT | ENST00000291495.5:c.2663_2664insCCGT | ENSP00000291495.3:p.Gln888HisfsTer180 | frameshift |
| 19:19545209:G:T | ENST00000291495.5:c.2664G > T | ENSP00000291495.3:p.Gln888His | missense |
| 19:19545211:G:A | ENST00000291495.5:c.2666G > A | ENSP00000291495.3:p.Gly889Glu | missense |
| 19:19545211:G:T | ENST00000291495.5:c.2666G > T | ENSP00000291495.3:p.Gly889Val | missense |
| 19:19545230:C:A | ENST00000291495.5:c.2685C > A | ENSP00000291495.3:p.Ser895Arg | missense |
| 19:19545237:C:T | ENST00000291495.5:c.2692C > T | ENSP00000291495.3:p.Arg898Cys | missense |
| 19:19545238:G:A | ENST00000291495.5:c.2693G > A | ENSP00000291495.3:p.Arg898His | missense |
| 19:19545238:G:T | ENST00000291495.5:c.2693G > T | ENSP00000291495.3:p.Arg898Leu | missense |
| 19:19545243:G:A | ENST00000291495.5:c.2698G > A | ENSP00000291495.3:p.Ala900Thr | missense |
| 19:19545243:G:T | ENST00000291495.5:c.2698G > T | ENSP00000291495.3:p.Ala900Ser | missense |
| 19:19545246:A:G | ENST00000291495.5:c.2701A > G | ENSP00000291495.3:p.Arg901Gly | missense |
| 19:19545246:AGG:A | ENST00000291495.5:c.2703_2704del | ENSP00000291495.3:p.Arg901SerfsTer165 | frameshift |
| 19:19545247:G:C | ENST00000291495.5:c.2702G > C | ENSP00000291495.3:p.Arg901Thr | missense |
| 19:19545250:T:A | ENST00000291495.5:c.2705T > A | ENSP00000291495.3:p.Val902Glu | missense |
| 19:19545250:T:C | ENST00000291495.5:c.2705T > C | ENSP00000291495.3:p.Val902Ala | missense |
| 19:19545255:G:A | ENST00000291495.5:c.2710G > A | ENSP00000291495.3:p.Ala904Thr | missense |
| 19:19545258:G:A | ENST00000291495.5:c.2713G > A | ENSP00000291495.3:p.Asp905Asn | missense |
| 19:19545265:A:G | ENST00000291495.5:c.2720A > G | ENSP00000291495.3:p.Tyr907Cys | missense |
| 19:19545266:C:A | ENST00000291495.5:c.2721C > A | ENSP00000291495.3:p.Tyr907Ter | stop_gained |
| 19:19545266:C:G | ENST00000291495.5:c.2721C > G | ENSP00000291495.3:p.Tyr907Ter | stop_gained |
| 19:19545267:G:A | ENST00000291495.5:c.2722G > A | ENSP00000291495.3:p.Glu908Lys | missense |
| 19:19545269:G:C | ENST00000291495.5:c.2724G > C | ENSP00000291495.3:p.Glu908Asp | missense |
| 19:19545271:A:G | ENST00000291495.5:c.2726A > G | ENSP00000291495.3:p.Tyr909Cys | missense |
| 19:19545272:C:A | ENST00000291495.5:c.2727C > A | ENSP00000291495.3:p.Tyr909Ter | stop_gained |
| 19:19545274:A:T | ENST00000291495.5:c.2729A > T | ENSP00000291495.3:p.Asn910Ile | missense |
| 19:19545276:G:A | ENST00000291495.5:c.2731G > A | ENSP00000291495.3:p.Val911Met | missense |
| 19:19545279:G:T | ENST00000291495.5:c.2734G > T | ENSP00000291495.3:p.Val912Phe | missense |
| 19:19545282:C:G | ENST00000291495.5:c.2737C > G | ENSP00000291495.3:p.Pro913Ala | missense |
| 19:19545288:C:T | ENST00000291495.5:c.2743C > T | ENSP00000291495.3:p.Arg915Ter | stop_gained |
| 19:19545289:G:A | ENST00000291495.5:c.2744G > A | ENSP00000291495.3:p.Arg915Gln | missense |
| 19:19545291:G:A | ENST00000291495.5:c.2746G > A | ENSP00000291495.3:p.Glu916Lys | missense |
| 19:19545295:G:T | ENST00000291495.5:c.2750G > T | ENSP00000291495.3:p.Gly917Val | missense |
| 19:19545300:C:G | ENST00000291495.5:c.2755C > G | ENSP00000291495.3:p.Pro919Ala | missense |
| 19:19545300:C:T | ENST00000291495.5:c.2755C > T | ENSP00000291495.3:p.Pro919Ser | missense |
| 19:19545301:C:T | ENST00000291495.5:c.2756C > T | ENSP00000291495.3:p.Pro919Leu | missense |
| 19:19545307:C:A | ENST00000291495.5:c.2762C > A | ENSP00000291495.3:p.Ser921Tyr | missense |
| 19:19545312:A:C | ENST00000291495.5:c.2767A > C | ENSP00000291495.3:p.Thr923Pro | missense |
| 19:19545313:C:T | ENST00000291495.5:c.2768C > T | ENSP00000291495.3:p.Thr923Ile | missense |
| 19:19545315:G:A | ENST00000291495.5:c.2770G > A | ENSP00000291495.3:p.Gly924Ser | missense |
| 19:19545316:G:A | ENST00000291495.5:c.2771G > A | ENSP00000291495.3:p.Gly924Asp | missense |
| 19:19545322:T:G | ENST00000291495.5:c.2777T > G | ENSP00000291495.3:p.Leu926Arg | missense |
| 19:19545333:T:A | ENST00000291495.5:c.2788T > A | ENSP00000291495.3:p.Trp930Arg | missense |
| 19:19545335:GCC:G | ENST00000291495.5:c.2792_2793del | ENSP00000291495.3:p.Pro931GlnfsTer135 | frameshift |
| 19:19545340:A:G | ENST00000291495.5:c.2795A > G | ENSP00000291495.3:p.Asn932Ser | missense |
| 19:19545348:G:A | ENST00000291495.5:c.2803G > A | ENSP00000291495.3:p.Glu935Lys | missense |
| 19:19545351:T:C | ENST00000291495.5:c.2806T > C | ENSP00000291495.3:p.Phe936Leu | missense |
| 19:19545355:G:A | ENST00000291495.5:c.2810G > A | ENSP00000291495.3:p.Arg937Gln | missense |
| 19:19545355:G:C | ENST00000291495.5:c.2810G > C | ENSP00000291495.3:p.Arg937Pro | missense |
| 19:19545355:G:T | ENST00000291495.5:c.2810G > T | ENSP00000291495.3:p.Arg937Leu | missense |
| 19:19545361:G:T | ENST00000291495.5:c.2816G > T | ENSP00000291495.3:p.Cys939Phe | missense |
| 19:19545365:C:A | ENST00000291495.5:c.2820C > A | ENSP00000291495.3:p.Phe940Leu | missense |
| 19:19545366:C:T | ENST00000291495.5:c.2821C > T | ENSP00000291495.3:p.Leu941Phe | missense |
| 19:19545372:G:A | ENST00000291495.5:c.2827G > A | ENSP00000291495.3:p.Val943Met | missense |
| 19:19545381:C:T | ENST00000291495.5:c.2836C > T | ENSP00000291495.3:p.Gln946Ter | stop_gained |
| 19:19545382:A:G | ENST00000291495.5:c.2837A > G | ENSP00000291495.3:p.Gln946Arg | missense |
| 19:19545385:G:A | ENST00000291495.5:c.2840G > A | ENSP00000291495.3:p.Gly947Asp | missense |
| 19:19545395:G:C | ENST00000291495.5:c.2850G > C | ENSP00000291495.3:p.Glu950Asp | missense |
| 19:19545398:T:A | ENST00000291495.5:c.2853T > A | ENSP00000291495.3:p.Tyr951Ter | stop_gained |
| 19:19545399:A:G | ENST00000291495.5:c.2854A > G | ENSP00000291495.3:p.Met952Val | missense |
| 19:19545402:G:A | ENST00000291495.5:c.2857G > A | ENSP00000291495.3:p.Val953Ile | missense |
| 19:19545405:C:T | ENST00000291495.5:c.2860C > T | ENSP00000291495.3:p.Arg954Cys | missense |
| 19:19545406:G:A | ENST00000291495.5:c.2861G > A | ENSP00000291495.3:p.Arg954His | missense |
| 19:19545409:C:T | ENST00000291495.5:c.2864C > T | ENSP00000291495.3:p.Ser955Phe | missense |
| 19:19545417:G:A | ENST00000291495.5:c.2872G > A | ENSP00000291495.3:p.Ala958Thr | missense |
| 19:19545417:G:T | ENST00000291495.5:c.2872G > T | ENSP00000291495.3:p.Ala958Ser | missense |
| 19:19545420:G:C | ENST00000291495.5:c.2875G > C | ENSP00000291495.3:p.Gly959Arg | missense |
| 19:19545424:G:A | ENST00000291495.5:c.2879G > A | ENSP00000291495.3:p.Gly960Asp | missense |
| 19:19545427:G:T | ENST00000291495.5:c.2882G > T | ENSP00000291495.3:p.Ser961Ile | missense |
| 19:19545429:C:G | ENST00000291495.5:c.2884C > G | ENSP00000291495.3:p.His962Asp | missense |
| 19:19545436:G:A | ENST00000291495.5:c.2891G > A | ENSP00000291495.3:p.Arg964His | missense |
| 19:19545441:C:G | ENST00000291495.5:c.2896C > G | ENSP00000291495.3:p.Arg966Gly | missense |
| 19:19545442:G:A | ENST00000291495.5:c.2897G > A | ENSP00000291495.3:p.Arg966His | missense |
| 19:19545444:G:A | ENST00000291495.5:c.2899G > A | ENSP00000291495.3:p.Gly967Ser | missense |
| 19:19545462:C:G | ENST00000291495.5:c.2917C > G | ENSP00000291495.3:p.Arg973Gly | missense |
| 19:19545463:G:C | ENST00000291495.5:c.2918G > C | ENSP00000291495.3:p.Arg973Pro | missense |
| 19:19545466:A:T | ENST00000291495.5:c.2921A > T | ENSP00000291495.3:p.Asp974Val | missense |
| 19:19545471:C:T | ENST00000291495.5:c.2926C > T | ENSP00000291495.3:p.Arg976Trp | missense |
| 19:19545472:G:A | ENST00000291495.5:c.2927G > A | ENSP00000291495.3:p.Arg976Gln | missense |
| 19:19545474:A:C | ENST00000291495.5:c.2929A > C | ENSP00000291495.3:p.Ser977Arg | missense |
| 19:19545476:T:G | ENST00000291495.5:c.2931T > G | ENSP00000291495.3:p.Ser977Arg | missense |
| 19:19545480:C:CA | ENST00000291495.5:c.2935_2936insA | ENSP00000291495.3:p.Arg979GlnfsTer88 | frameshift |
| 19:19545485:C:A | ENST00000291495.5:c.2940C > A | ENSP00000291495.3:p.Asp980Glu | missense |
| 19:19545486:C:A | ENST00000291495.5:c.2941C > A | ENSP00000291495.3:p.Pro981Thr | missense |
| 19:19545489:G:A | ENST00000291495.5:c.2944G > A | ENSP00000291495.3:p.Glu982Lys | missense |
| 19:19545492:C:T | ENST00000291495.5:c.2947C > T | ENSP00000291495.3:p.Arg983Cys | missense |
| 19:19545493:G:A | ENST00000291495.5:c.2948G > A | ENSP00000291495.3:p.Arg983His | missense |
| 19:19545496:C:T | ENST00000291495.5:c.2951C > T | ENSP00000291495.3:p.Pro984Leu | missense |
| 19:19545499:G:C | ENST00000291495.5:c.2954G > C | ENSP00000291495.3:p.Gly985Ala | missense |
| 19:19545505:C:G | ENST00000291495.5:c.2960C > G | ENSP00000291495.3:p.Ser987Trp | missense |
| 19:19545508:C:G | ENST00000291495.5:c.2963C > G | ENSP00000291495.3:p.Ala988Gly | missense |
| 19:19545515:C:A | ENST00000291495.5:c.2970C > A | ENSP00000291495.3:p.Cys990Ter | stop_gained |
| 19:19545516:G:A | ENST00000291495.5:c.2971G > A | ENSP00000291495.3:p.Val991Met | missense |
| 19:19545517:T:G | ENST00000291495.5:c.2972T > G | ENSP00000291495.3:p.Val991Gly | missense |
| 19:19545527:G:C | ENST00000291495.5:c.2982G > C | ENSP00000291495.3:p.Lys994Asn | missense |
| 19:19545529:G:T | ENST00000291495.5:c.2984G > T | ENSP00000291495.3:p.Cys995Phe | missense |
| 19:19545534:G:A | ENST00000291495.5:c.2989G > A | ENSP00000291495.3:p.Gly997Arg | missense |
| 19:19545538:T:C | ENST00000291495.5:c.2993T > C | ENSP00000291495.3:p.Met998Thr | missense |
| 19:19545545:C:G | ENST00000291495.5:c.3000C > G | ENSP00000291495.3:p.Phe1000Leu | missense |
| 19:19545546:G:A | ENST00000291495.5:c.3001G > A | ENSP00000291495.3:p.Asp1001Asn | missense |
| 19:19545546:G:T | ENST00000291495.5:c.3001G > T | ENSP00000291495.3:p.Asp1001Tyr | missense |
| 19:19545550:A:G | ENST00000291495.5:c.3005A > G | ENSP00000291495.3:p.Gln1002Arg | missense |
| 19:19545564:A:G | ENST00000291495.5:c.3019A > G | ENSP00000291495.3:p.Arg1007Gly | missense |
| 19:19545571:T:A | ENST00000291495.5:c.3026T > A | ENSP00000291495.3:p.Leu1009Gln | missense |
| 19:19545573:G:A | ENST00000291495.5:c.3028G > A | ENSP00000291495.3:p.Val1010Met | missense |
| 19:19545574:T:C | ENST00000291495.5:c.3029T > C | ENSP00000291495.3:p.Val1010Ala | missense |
| 19:19545585:C:A | ENST00000291495.5:c.3040C > A | ENSP00000291495.3:p.Pro1014Thr | missense |
| 19:19545586:C:A | ENST00000291495.5:c.3041C > A | ENSP00000291495.3:p.Pro1014His | missense |
| 19:19545588:CAGGG:C | ENST00000291495.5:c.3044_3047del | ENSP00000291495.3:p.Gln1015ProfsTer16 | frameshift |
| 19:19545590:G:C | ENST00000291495.5:c.3045G > C | ENSP00000291495.3:p.Gln1015His | missense |
| 19:19545601:G:A | ENST00000291495.5:c.3056G > A | ENSP00000291495.3:p.Arg1019Gln | missense |
| 19:19545603:C:T | ENST00000291495.5:c.3058C > T | ENSP00000291495.3:p.Arg1020Cys | missense |
| 19:19545604:G:A | ENST00000291495.5:c.3059G > A | ENSP00000291495.3:p.Arg1020His | missense |
| 19:19545604:G:T | ENST00000291495.5:c.3059G > T | ENSP00000291495.3:p.Arg1020Leu | missense |
| 19:19545606:G:A | ENST00000291495.5:c.3061G > A | ENSP00000291495.3:p.Val1021Met | missense |
| 19:19545606:G:C | ENST00000291495.5:c.3061G > C | ENSP00000291495.3:p.Val1021Leu | missense |
| 19:19545607:T:A | ENST00000291495.5:c.3062T > A | ENSP00000291495.3:p.Val1021Glu | missense |
| 19:19545612:G:A | ENST00000291495.5:c.3067G > A | ENSP00000291495.3:p.Val1023Ile | missense |
| 19:19545612:G:C | ENST00000291495.5:c.3067G > C | ENSP00000291495.3:p.Val1023Leu | missense |
| 19:19545618:G:A | ENST00000291495.5:c.3073G > A | ENSP00000291495.3:p.Gly1025Arg | missense |
| 19:19545618:G:T | ENST00000291495.5:c.3073G > T | ENSP00000291495.3:p.Gly1025Ter | stop_gained |
| 19:19545619:G:T | ENST00000291495.5:c.3074G > T | ENSP00000291495.3:p.Gly1025Val | missense |
| 19:19545621:C:A | ENST00000291495.5:c.3076C > A | ENSP00000291495.3:p.Leu1026Ile | missense |
| 19:19545622:T:G | ENST00000291495.5:c.3077T > G | ENSP00000291495.3:p.Leu1026Arg | missense |
| 19:19545627:C:T | ENST00000291495.5:c.3082C > T | ENSP00000291495.3:p.Arg1028Trp | missense |
| 19:19545630:G:A | ENST00000291495.5:c.3085G > A | ENSP00000291495.3:p.Asp1029Asn | missense |
| 19:19545642:C:T | ENST00000291495.5:c.3097C > T | ENSP00000291495.3:p.Arg1033Trp | missense |
| 19:19545643:G:A | ENST00000291495.5:c.3098G > A | ENSP00000291495.3:p.Arg1033Gln | missense |
| 19:19545643:G:T | ENST00000291495.5:c.3098G > T | ENSP00000291495.3:p.Arg1033Leu | missense |
| 19:19545646:A:G | ENST00000291495.5:c.3101A > G | ENSP00000291495.3:p.His1034Arg | missense |
| 19:19545647:C:A | ENST00000291495.5:c.3102C > A | ENSP00000291495.3:p.His1034Gln | missense |
| 19:19545652:C:A | ENST00000291495.5:c.3107C > A | ENSP00000291495.3:p.Pro1036Gln | missense |
| 19:19545652:C:G | ENST00000291495.5:c.3107C > G | ENSP00000291495.3:p.Pro1036Arg | missense |
| 19:19545652:C:T | ENST00000291495.5:c.3107C > T | ENSP00000291495.3:p.Pro1036Leu | missense |
| 19:19545663:G:A | ENST00000291495.5:c.3118G > A | ENSP00000291495.3:p.Ala1040Thr | missense |
| 19:19545664:C:T | ENST00000291495.5:c.3119C > T | ENSP00000291495.3:p.Ala1040Val | missense |
| 19:19545669:G:A | ENST00000291495.5:c.3124G > A | ENSP00000291495.3:p.Asp1042Asn | missense |
| 19:19545672:C:T | ENST00000291495.5:c.3127C > T | ENSP00000291495.3:p.Pro1043Ser | missense |
| 19:19545676:CT:C | ENST00000291495.5:c.3132del | ENSP00000291495.3:p.Ala1045ProfsTer8 | frameshift |
| 19:19545678:G:A | ENST00000291495.5:c.3133G > A | ENSP00000291495.3:p.Ala1045Thr | missense |
| 19:19545679:C:T | ENST00000291495.5:c.3134C > T | ENSP00000291495.3:p.Ala1045Val | missense |
| 19:19545693:GC:G | ENST00000291495.5:c.3154del | ENSP00000291495.3:p.Leu1052Ter | frameshift |
| 19:19545694:C:A | ENST00000291495.5:c.3149C > A | ENSP00000291495.3:p.Ala1050Asp | missense |
| 19:19545694:C:G | ENST00000291495.5:c.3149C > G | ENSP00000291495.3:p.Ala1050Gly | missense |
| 19:19545696:C:A | ENST00000291495.5:c.3151C > A | ENSP00000291495.3:p.Pro1051Thr | missense |
| 19:19545697:C:T | ENST00000291495.5:c.3152C > T | ENSP00000291495.3:p.Pro1051Leu | missense |
| 19:19545706:C:G | ENST00000291495.5:c.3161C > G | ENSP00000291495.3:p.Pro1054Arg | missense |
| 19:19545706:C:T | ENST00000291495.5:c.3161C > T | ENSP00000291495.3:p.Pro1054Leu | missense |
| 19:19545717:A:G | ENST00000291495.5:c.3172A > G | ENSP00000291495.3:p.Asn1058Asp | missense |
| 19:19545720:T:C | ENST00000291495.5:c.3175T > C | ENSP00000291495.3:p.Tyr1059His | missense |
| 19:19545726:G:A | ENST00000291495.5:c.3181G > A | ENSP00000291495.3:p.Val1061Ile | missense |
| 19:19545729:T:C | ENST00000291495.5:c.3184T > C | ENSP00000291495.3:p.Tyr1062His | missense |
| 19:19545730:A:G | ENST00000291495.5:c.3185A > G | ENSP00000291495.3:p.Tyr1062Cys | missense |
| 19:19545732:A:T | ENST00000291495.5:c.3187A > T | ENSP00000291495.3:p.Thr1063Ser | missense |
| 19:19545733:CTG:C | ENST00000291495.5:c.3190_3191del | ENSP00000291495.3:p.Val1064HisfsTer2 | frameshift |
| 19:19545735:G:A | ENST00000291495.5:c.3190G > A | ENSP00000291495.3:p.Val1064Ile | missense |
| 19:19545739:C:G | ENST00000291495.5:c.3194C > G | ENSP00000291495.3:p.Thr1065Ser | missense |
| 19:19545745:A:G | ENST00000291495.5:c.3200A > G | ENSP00000291495.3:p.Gln1067Arg | missense |
| 19:19545747:A:G | ENST00000291495.5:c.3202A > G | ENSP00000291495.3:p.Ser1068Gly | missense |
| 19:19545748:G:A | ENST00000291495.5:c.3203G > A | ENSP00000291495.3:p.Ser1068Asn | missense |
| 19:19545753:C:T | ENST00000291495.5:c.3208C > T | ENSP00000291495.3:p.Arg1070Cys | missense |
| 19:19545754:G:A | ENST00000291495.5:c.3209G > A | ENSP00000291495.3:p.Arg1070His | missense |
| 19:19545754:G:C | ENST00000291495.5:c.3209G > C | ENSP00000291495.3:p.Arg1070Pro | missense |
| 19:19545771:G:A | ENST00000291495.5:c.3226G > A | ENSP00000291495.3:p.Ala1076Thr | missense |
| 19:19545772:C:T | ENST00000291495.5:c.3227C > T | ENSP00000291495.3:p.Ala1076Val | missense |
| 19:19545775:T:C | ENST00000291495.5:c.3230T > C | ENSP00000291495.3:p.Ile1077Thr | missense |
| 19:19545780:C:A | ENST00000291495.5:c.3235C > A | ENSP00000291495.3:p.Arg1079Ser | missense |
| 19:19545780:C:T | ENST00000291495.5:c.3235C > T | ENSP00000291495.3:p.Arg1079Cys | missense |
| 19:19545781:G:A | ENST00000291495.5:c.3236G > A | ENSP00000291495.3:p.Arg1079His | missense |
| 19:19545790:A:G | ENST00000291495.5:c.3245A > G | ENSP00000291495.3:p.Asp1082Gly | missense |
| 19:19545801:G:A | ENST00000291495.5:c.3256G > A | ENSP00000291495.3:p.Asp1086Asn | missense |
| 19:19545803:C:G | ENST00000291495.5:c.3258C > G | ENSP00000291495.3:p.Asp1086Glu | missense |
| 19:19545804:G:A | ENST00000291495.5:c.3259G > A | ENSP00000291495.3:p.Gly1087Ser | missense |
| 19:19545804:G:C | ENST00000291495.5:c.3259G > C | ENSP00000291495.3:p.Gly1087Arg | missense |
| 19:19545809:C:A | ENST00000291495.5:c.3264C > A | ENSP00000291495.3:p.Phe1088Leu | missense |
| 19:19545811:C:A | ENST00000291495.5:c.3266C > A | ENSP00000291495.3:p.Ser1089Tyr | missense |
| 19:19545814:G:C | ENST00000291495.5:c.3269G > C | ENSP00000291495.3:p.Arg1090Thr | missense |
| 19:19545820:T:C | ENST00000291495.5:c.3275T > C | ENSP00000291495.3:p.Met1092Thr | missense |
| 19:19545829:A:T | ENST00000291495.5:c.3284A > T | ENSP00000291495.3:p.Asp1095Val | missense |
| 19:19545832:C:T | ENST00000291495.5:c.3287C > T | ENSP00000291495.3:p.Ala1096Val | missense |
| 19:19545834:G:A | ENST00000291495.5:c.3289G > A | ENSP00000291495.3:p.Gly1097Ser | missense |
| 19:19545834:G:C | ENST00000291495.5:c.3289G > C | ENSP00000291495.3:p.Gly1097Arg | missense |
| 19:19545838:C:T | ENST00000291495.5:c.3293C > T | ENSP00000291495.3:p.Thr1098Ile | missense |
| 19:19545843:G:A | ENST00000291495.5:c.3298G > A | ENSP00000291495.3:p.Val1100Ile | missense |
| 19:19545843:G:C | ENST00000291495.5:c.3298G > C | ENSP00000291495.3:p.Val1100Leu | missense |
| 19:19545844:T:G | ENST00000291495.5:c.3299T > G | ENSP00000291495.3:p.Val1100Gly | missense |
| 19:19545852:C:T | ENST00000291495.5:c.3307C > T | ENSP00000291495.3:p.Gln1103Ter | stop_gained |
| 19:19545859:G:A | ENST00000291495.5:c.3314G > A | ENSP00000291495.3:p.Arg1105Gln | missense |
| 19:19545864:C:G | ENST00000291495.5:c.3319C > G | ENSP00000291495.3:p.Pro1107Ala | missense |
| 19:19545864:C:T | ENST00000291495.5:c.3319C > T | ENSP00000291495.3:p.Pro1107Ser | missense |
| 19:19545867:C:A | ENST00000291495.5:c.3322C > A | ENSP00000291495.3:p.Pro1108Thr | missense |
| 19:19545867:C:G | ENST00000291495.5:c.3322C > G | ENSP00000291495.3:p.Pro1108Ala | missense |
| 19:19545868:C:T | ENST00000291495.5:c.3323C > T | ENSP00000291495.3:p.Pro1108Leu | missense |
| 19:19545870:G:A | ENST00000291495.5:c.3325G > A | ENSP00000291495.3:p.Ala1109Thr | missense |
| 19:19545873:G:A | ENST00000291495.5:c.3328G > A | ENSP00000291495.3:p.Gly1110Arg | missense |
| 19:19545876:C:T | ENST00000291495.5:c.3331C > T | ENSP00000291495.3:p.Arg1111Ter | stop_gained |
| 19:19545882:A:C | ENST00000291495.5:c.3337A > C | ENSP00000291495.3:p.Ser1113Arg | missense |
| 19:19545883:G:A | ENST00000291495.5:c.3338G > A | ENSP00000291495.3:p.Ser1113Asn | missense |
| 19:19545884:C:A | ENST00000291495.5:c.3339C > A | ENSP00000291495.3:p.Ser1113Arg | missense |
| 19:19545885:C:T | ENST00000291495.5:c.3340C > T | ENSP00000291495.3:p.Leu1114Phe | missense |
| 19:19545886:T:G | ENST00000291495.5:c.3341T > G | ENSP00000291495.3:p.Leu1114Arg | missense |
| 19:19545895:G:A | ENST00000291495.5:c.3350G > A | ENSP00000291495.3:p.Arg1117Lys | missense |
| 19:19545895:G:C | ENST00000291495.5:c.3350G > C | ENSP00000291495.3:p.Arg1117Thr | missense |
| 19:19545905:G:T | ENST00000291495.5:c.3360G > T | ENSP00000291495.3:p.Glu1120Asp | missense |
| 19:19545906:T:A | ENST00000291495.5:c.3361T > A | ENSP00000291495.3:p.Ser1121Thr | missense |
| 19:19545906:T:G | ENST00000291495.5:c.3361T > G | ENSP00000291495.3:p.Ser1121Ala | missense |
| 19:19545907:C:A | ENST00000291495.5:c.3362C > A | ENSP00000291495.3:p.Ser1121Tyr | missense |
| 19:19545909:C:T | ENST00000291495.5:c.3364C > T | ENSP00000291495.3:p.Pro1122Ser | missense |
| 19:19545910:C:G | ENST00000291495.5:c.3365C > G | ENSP00000291495.3:p.Pro1122Arg | missense |
| 19:19545910:C:T | ENST00000291495.5:c.3365C > T | ENSP00000291495.3:p.Pro1122Leu | missense |
| 19:19545913:C:T | ENST00000291495.5:c.3368C > T | ENSP00000291495.3:p.Ala1123Val | missense |
| 19:19545921:C:T | ENST00000291495.5:c.3376C > T | ENSP00000291495.3:p.Leu1126Phe | missense |
| 19:19545925:G:A | ENST00000291495.5:c.3380G > A | ENSP00000291495.3:p.Gly1127Asp | missense |
| 19:19545925:G:C | ENST00000291495.5:c.3380G > C | ENSP00000291495.3:p.Gly1127Ala | missense |
| 19:19545927:G:A | ENST00000291495.5:c.3382G > A | ENSP00000291495.3:p.Asp1128Asn | missense |
| 19:19545930:A:G | ENST00000291495.5:c.3385A > G | ENSP00000291495.3:p.Ile1129Val | missense |
| 19:19545933:C:A | ENST00000291495.5:c.3388C > A | ENSP00000291495.3:p.Arg1130Ser | missense |
| 19:19545933:C:G | ENST00000291495.5:c.3388C > G | ENSP00000291495.3:p.Arg1130Gly | missense |
| 19:19545933:C:T | ENST00000291495.5:c.3388C > T | ENSP00000291495.3:p.Arg1130Cys | missense |
| 19:19545934:G:A | ENST00000291495.5:c.3389G > A | ENSP00000291495.3:p.Arg1130His | missense |
| 19:19545934:G:T | ENST00000291495.5:c.3389G > T | ENSP00000291495.3:p.Arg1130Leu | missense |
| 19:19545939:G:A | ENST00000291495.5:c.3394G > A | ENSP00000291495.3:p.Glu1132Lys | missense |
| 19:19545944:G:A | ENST00000291495.5:c.3399G > A | ENSP00000291495.3:p.Met1133Ile | missense |
| 19:19545944:G:T | ENST00000291495.5:c.3399G > T | ENSP00000291495.3:p.Met1133Ile | missense |
| 19:19545948:G:A | ENST00000291495.5:c.3403G > A | ENSP00000291495.3:p.Glu1135Lys | missense |
| 19:19545948:G:C | ENST00000291495.5:c.3403G > C | ENSP00000291495.3:p.Glu1135Gln | missense |
| 19:19545951:G:A | ENST00000291495.5:c.3406G > A | ENSP00000291495.3:p.Ala1136Thr | missense |
| 19:19545952:C:T | ENST00000291495.5:c.3407C > T | ENSP00000291495.3:p.Ala1136Val | missense |
| 19:19545964:A:G | ENST00000291495.5:c.3419A > G | ENSP00000291495.3:p.Gln1140Arg | missense |
| 19:19545969:C:T | ENST00000291495.5:c.3424C > T | ENSP00000291495.3:p.Arg1142Trp | missense |
| 19:19545970:G:A | ENST00000291495.5:c.3425G > A | ENSP00000291495.3:p.Arg1142Gln | missense |
| 19:19545981:C:T | ENST00000291495.5:c.3436C > T | ENSP00000291495.3:p.Pro1146Ser | missense |
| 19:19545987:C:G | ENST00000291495.5:c.3442C > G | ENSP00000291495.3:p.Arg1148Gly | missense |
| 19:19545987:C:T | ENST00000291495.5:c.3442C > T | ENSP00000291495.3:p.Arg1148Cys | missense |
| 19:19545988:G:A | ENST00000291495.5:c.3443G > A | ENSP00000291495.3:p.Arg1148His | missense |
| 19:19545993:C:G | ENST00000291495.5:c.3448C > G | ENSP00000291495.3:p.Arg1150Gly | missense |
| 19:19545993:C:T | ENST00000291495.5:c.3448C > T | ENSP00000291495.3:p.Arg1150Cys | missense |
| 19:19545994:G:A | ENST00000291495.5:c.3449G > A | ENSP00000291495.3:p.Arg1150His | missense |
| 19:19545994:G:C | ENST00000291495.5:c.3449G > C | ENSP00000291495.3:p.Arg1150Pro | missense |
| 19:19545996:C:G | ENST00000291495.5:c.3451C > G | ENSP00000291495.3:p.Arg1151Gly | missense |
| 19:19545996:C:T | ENST00000291495.5:c.3451C > T | ENSP00000291495.3:p.Arg1151Trp | missense |
| 19:19545997:G:A | ENST00000291495.5:c.3452G > A | ENSP00000291495.3:p.Arg1151Gln | missense |
| 19:19545999:G:T | ENST00000291495.5:c.3454G > T | ENSP00000291495.3:p.Gly1152Cys | missense |
| 19:19546000:G:T | ENST00000291495.5:c.3455G > T | ENSP00000291495.3:p.Gly1152Val | missense |
| 19:19546004:G:C | ENST00000291495.5:c.3459G > C | ENSP00000291495.3:p.Arg1153Ser | missense |
| 19:19546005:G:A | ENST00000291495.5:c.3460G > A | ENSP00000291495.3:p.Val1154Ile | missense |
| 19:19546008:C:T | ENST00000291495.5:c.3463C > T | ENSP00000291495.3:p.Arg1155Trp | missense |
| 19:19546009:G:A | ENST00000291495.5:c.3464G > A | ENSP00000291495.3:p.Arg1155Gln | missense |
| 19:19546009:G:C | ENST00000291495.5:c.3464G > C | ENSP00000291495.3:p.Arg1155Pro | missense |
| 19:19546016:A:T | ENST00000291495.5:c.3471A > T | ENSP00000291495.3:p.Ter1157CysextTer88 | stop_lost |

Table 2 contains statistics for both the joint space analysis and the strongest diagnosis-based OA analysis association for CILP2 (knee). In particular, displayed are all pLoF and missense variants detected via exome sequencing of CILP2. CPRA indicates the genomic coordinates: chromosome, physical genomic position in base pairs, reference allele, and alternative allele for each variant, according to build 38 of the Human Genome sequence by the Human Genome Reference Consortium (see, Table 1). Single variant association analysis results are reported for each variant based on a trans-ancestral meta-analysis; if sample sizes are insufficient to make an estimation (e.g., a variant is too rare), the result is listed as “NA.” For columns with reported effect sizes, OR indicates odds ratio and SD indicates standard deviation units along with the corresponding 95% confidence interval. AAF indicates the alternative allele frequency as a fraction of 1. In all cases, predicted impacts of variation are based on the canonical CILP2 transcript annotated in the in the Ensembl database (ENST00000291495; see, world wide web at “useast.ensembl.org/index.html”).

TABLE 2

| | Joint Space Analysis | | | Diagnosis-based knee OA analysis | |
|---|---|---|---|---|---|
| | Effect (95% CI) | | | Effect (95% CI) | |
| CPRA | per allele in SD | P-value | AAF | per allele as OR | P-value |
| 19:19538359:C:G | NA | NA | NA | 7.844 | 0.009373 |
| | | | | (1.659, 37.102) | |
| 19:19538361:G:GA | NA | NA | NA | 0.274 | 0.2155 |
| | | | | (0.035, 2.125) | |
| 19:19538379:CTG:C | 0.567 | 0.00360875 | 0.000238273 | 0.564 | 0.0274 |
| | (0.185, 0.949) | | | (0.339, 0.938) | |
| 19:19539679:A:T | NA | NA | NA | 0.291 | 0.3242 |
| | | | | (0.025, 3.382) | |
| 19:19539682:C:T | NA | NA | NA | 2.608 | 0.05342 |
| | | | | (0.986, 6.897) | |
| 19:19539688:C:T | NA | NA | NA | 3.136 | 0.04324 |
| | | | | (1.035, 9.498) | |
| 19:19539693:G:A | NA | NA | NA | 2.657 | 0.05493 |
| | | | | (0.979, 7.206) | |
| 19:19539717:G:A | NA | NA | NA | 0.254 | 0.0627 |
| | | | | (0.06, 1.075) | |
| 19:19539744:G:A | NA | NA | NA | 0.48 | 0.3259 |
| | | | | (0.111, 2.076) | |
| 19:19539772:G:C | NA | NA | NA | 0.296 | 0.3189 |
| | | | | (0.027, 3.248) | |
| 19:19540205:G:T | −0.026 | 0.943277 | 6.22E−05 | 0.633 | 0.3858 |
| | (−0.773, 0.72) | | | (0.225, 1.78) | |
| 19:19540212:G:C | NA | NA | NA | 3.25 | 0.3053 |
| | | | | (0.341, 30.944) | |
| 19:19540228:T:A | 0.411 | 0.281504 | 6.22E−05 | 0.517 | 0.3653 |
| | (−0.336, 1.158) | | | (0.124, 2.155) | |
| 19:19540233:G:C | NA | NA | NA | 0.32 | 0.5199 |
| | | | | (0.01, 10.29) | |
| 19:19540239:C:A | −0.048 | 0.897443 | 6.22E−05 | 1.36 | 0.7206 |
| | (−0.795, 0.698) | | | (0.252, 7.349) | |
| 19:19540249:G:A | NA | NA | NA | 0.885 | 0.8114 |
| | | | | (0.323, 2.422) | |
| 19:19540272:G:A | −0.15 | 0.762088 | 6.00E−05 | NA | NA |
| | (−1.125, 0.825) | | | | |
| 19:19540281:C:T | NA | NA | NA | 0.301 | 0.3552 |
| | | | | (0.024, 3.837) | |
| 19:19540306:T:G | NA | NA | NA | 0.406 | 0.2348 |
| | | | | (0.092, 1.796) | |
| 19:19540365:G:A | NA | NA | NA | 0.877 | 0.9004 |
| | | | | (0.113, 6.825) | |
| 19:19540374:C:T | −0.168 | 0.0434136 | 0.00129491 | 1.005 | 0.9539 |
| | (−0.332, −0.004) | | | (0.855, 1.181) | |
| 19:19540387:A:T | −0.171 | 0.648555 | 7.02E−05 | 0.872 | 0.8511 |
| | (−0.91, 0.567) | | | (0.209, 3.64) | |
| 19:19540390:C:T | NA | NA | NA | 0.718 | 0.6244 |
| | | | | (0.19, 2.708) | |
| 19:19540426:C:G | NA | NA | NA | 0.766 | 0.7072 |
| | | | | (0.191, 3.078) | |
| 19:19540437:C:G | NA | NA | NA | 0.704 | 0.5874 |
| | | | | (0.198, 2.502) | |
| 19:19540440:T:C | NA | NA | NA | 1.048 | 0.9668 |
| | | | | (0.115, 9.517) | |
| 19:19540446:A:C | NA | NA | NA | 4.654 | 0.02018 |
| | | | | (1.272, 17.035) | |
| 19:19540447:A:C | 0.206 | 0.563681 | 7.25E−05 | 1.637 | 0.3996 |
| | (−0.492, 0.905) | | | (0.52, 5.155) | |
| 19:19540464:C:G | NA | NA | NA | 6.217 | 0.05537 |
| | | | | (0.959, 40.307) | |
| 19:19541093:G:T | NA | NA | NA | 3.674 | 0.2547 |
| | | | | (0.391, 34.483) | |
| 19:19541097:C:T | NA | NA | NA | 0.32 | 0.474 |
| | | | | (0.014, 7.25) | |
| 19:19541124:C:T | NA | NA | NA | 0.502 | 0.1824 |
| | | | | (0.182, 1.383) | |
| 19:19541148:G:A | NA | NA | NA | 0.404 | 0.2705 |
| | | | | (0.081, 2.026) | |
| 19:19541175:C:T | NA | NA | NA | 0.724 | 0.7254 |
| | | | | (0.119, 4.4) | |
| 19:19541183:G:A | NA | NA | NA | 0.645 | 0.6072 |
| | | | | (0.121, 3.434) | |
| 19:19541216:G:T | NA | NA | NA | 0.33 | 0.4265 |
| | | | | (0.021, 5.08) | |
| 19:19542393:G:A | NA | NA | NA | 1.752 | 0.6064 |
| | | | | (0.208, 14.778) | |
| 19:19542402:C:T | 0.307 | 0.275911 | 0.000113949 | 1.245 | 0.5103 |
| | (−0.244, 0.859) | | | (0.649, 2.387) | |
| 19:19542417:T:C | NA | NA | NA | 1.84 | 0.6404 |
| | | | | (0.142, 23.761) | |
| 19:19542422:T:A | NA | NA | NA | 0.986 | 0.9515 |
| | | | | (0.623, 1.56) | |
| 19:19542423:C:T | NA | NA | NA | 0.939 | 0.955 |
| | | | | (0.104, 8.508) | |
| 19:19542485:G:C | NA | NA | NA | 1.205 | 0.7425 |
| | | | | (0.396, 3.663) | |
| 19:19542521:C:T | −0.165 | 0.49133 | 0.000155386 | 0.835 | 0.4418 |
| | (−0.638, 0.307) | | | (0.528, 1.322) | |
| 19:19542522:G:A | NA | NA | NA | 0.941 | 0.9259 |
| | | | | (0.26, 3.399) | |
| 19:19542548:C:T | NA | NA | NA | 0.86 | 0.7051 |
| | | | | (0.394, 1.877) | |
| 19:19542549:G:A | NA | NA | NA | 3.53 | 0.2043 |
| | | | | (0.503, 24.745) | |
| 19:19542570:T:C | NA | NA | NA | 0.858 | 0.8846 |
| | | | | (0.109, 6.782) | |
| 19:19542584:G:T | NA | NA | NA | 0.319 | 0.3318 |
| | | | | (0.032, 3.201) | |
| 19:19542593:G:A | NA | NA | NA | 2.486 | 0.4107 |
| | | | | (0.284, 21.764) | |
| 19:19542632:A:T | NA | NA | NA | 0.781 | 0.8055 |
| | | | | (0.109, 5.59) | |
| 19:19542896:C:T | NA | NA | NA | 1.226 | 0.8704 |
| | | | | (0.106, 14.228) | |
| 19:19542902:C:G | NA | NA | NA | 3.233 | 0.7805 |
| | | | | (0.001, 12388.9) | |
| 19:19542908:G:T | NA | NA | NA | 1.476 | 0.2127 |
| | | | | (0.8, 2.721) | |
| 19:19542939:C:T | NA | NA | NA | 0.508 | 0.346 |
| | | | | (0.125, 2.075) | |
| 19:19542944:G:A | 0.274 | 0.135162 | 0.000269335 | 1.256 | 0.3318 |
| | (−0.084, 0.633) | | | (0.793, 1.99) | |
| 19:19543256:A:T | NA | NA | NA | 0.32 | 0.4306 |
| | | | | (0.019, 5.435) | |
| 19:19543277:G:A | 0.37 | 0.102405 | 0.000176104 | 0.397 | 0.03266 |
| | (−0.073, 0.814) | | | (0.17, 0.927) | |
| 19:19543279:G:C | 0.02 | 0.752871 | 0.0023515 | 0.922 | 0.1624 |
| | (−0.101, 0.141) | | | (0.823, 1.033) | |
| 19:19543291:G:A | NA | NA | NA | 4.515 | 0.1886 |
| | | | | (0.477, 42.721) | |
| 19:19543305:G:T | NA | NA | NA | 3.702 | 0.169 |
| | | | | (0.573, 23.901) | |
| 19:19543309:C:T | NA | NA | NA | 0.465 | 0.274 |
| | | | | (0.118, 1.833) | |
| 19:19543310:G:A | 0.556 | 0.219654 | 8.35E−05 | 1.109 | 0.8006 |
| | (−0.331, 1.443) | | | (0.495, 2.484) | |
| 19:19543318:C:T | 0.098 | 0.629971 | 0.00021754 | 1.231 | 0.4428 |
| | (−0.3, 0.498) | | | (0.724, 2.091) | |
| 19:19543319:G:A | NA | NA | NA | 64.335 | 0.0685 |
| | | | | (0.729, 5678.15) | |
| 19:19543330:G:T | NA | NA | NA | 3.835 | 0.08939 |
| | | | | (0.813, 18.086) | |
| 19:19543372:G:A | 0.238 | 0.358004 | 0.000134668 | 1.144 | 0.7496 |
| | (−0.269, 0.746) | | | (0.501, 2.615) | |
| 19:19543375:C:T | NA | NA | NA | 1.09 | 0.9139 |
| | | | | (0.229, 5.177) | |
| 19:19543376:G:A | 0.472 | 0.0937363 | 0.000113949 | 0.723 | 0.4466 |
| | (−0.079, 1.024) | | | (0.314, 1.667) | |
| 19:19543379:C:T | NA | NA | NA | 15.914 | 0.05218 |
| | | | | (0.974, 259.963) | |
| 19:19543390:C:T | −0.18 | 0.561954 | 9.32E−05 | 0.601 | 0.3798 |
| | (−0.79, 0.43) | | | (0.193, 1.871) | |
| 19:19543391:G:A | 0.061 | 0.542891 | 0.000890878 | 1.03 | 0.8053 |
| | (−0.135, 0.259) | | | (0.815, 1.302) | |
| 19:19543699:G:T | NA | NA | NA | 0.501 | 0.3387 |
| | | | | (0.122, 2.063) | |
| 19:19543708:G:A | 0.037 | 0.56613 | 0.00220648 | 0.962 | 0.501 |
| | (−0.088, 0.163) | | | (0.859, 1.077) | |
| 19:19543713:C:G | NA | NA | NA | 1.324 | 0.6375 |
| | | | | (0.412, 4.249) | |
| 19:19543713:C:T | NA | NA | NA | 0.278 | 0.3455 |
| | | | | (0.019, 3.976) | |
| 19:19543714:G:A | NA | NA | NA | 0.934 | 0.9519 |
| | | | | (0.1, 8.685) | |
| 19:19543716:G:T | NA | NA | NA | 0.822 | 0.8489 |
| | | | | (0.11, 6.158) | |
| 19:19543726:T:C | NA | NA | NA | 0.917 | 0.889 |
| | | | | (0.273, 3.084) | |
| 19:19543794:G:A | NA | NA | NA | 0.283 | 0.3651 |
| | | | | (0.019, 4.339) | |
| 19:19543795:A:C | NA | NA | NA | 0.314 | 0.3303 |
| | | | | (0.03, 3.238) | |
| 19:19543800:C:G | NA | NA | NA | 1.84 | 0.5255 |
| | | | | (0.28, 12.081) | |
| 19:19543801:G:A | −0.467 | 0.256375 | 0.000132572 | 0.323 | 0.1457 |
| | (−1.275, 0.34) | | | (0.071, 1.48) | |
| 19:19543819:G:T | NA | NA | NA | 1.743 | 0.3425 |
| | | | | (0.553, 5.493) | |
| 19:19543830:C:A | −0.229 | 0.582499 | 5.18E−05 | 0.377 | 0.0565 |
| | (−1.047, 0.589) | | | (0.139, 1.027) | |
| 19:19543830:C:T | NA | NA | NA | 0.311 | 0.3027 |
| | | | | (0.034, 2.868) | |
| 19:19543842:G:A | NA | NA | NA | 0.842 | 0.8209 |
| | | | | (0.19, 3.729) | |
| 19:19543852:G:C | NA | NA | NA | 2.844 | 0.4826 |
| | | | | (0.154, 52.63) | |
| 19:19543869:C:T | NA | NA | NA | 1.712 | 0.5221 |
| | | | | (0.33, 8.878) | |
| 19:19543870:G:A | NA | NA | NA | 0.715 | 0.6395 |
| | | | | (0.175, 2.913) | |
| 19:19543900:G:A | NA | NA | NA | 0.339 | 0.5266 |
| | | | | (0.012, 9.666) | |
| 19:19543953:C:T | −0.399 | 0.338062 | 5.18E−05 | 0.907 | 0.7756 |
| | (−1.218, 0.418) | | | (0.465, 1.77) | |
| 19:19543954:C:T | NA | NA | NA | 0.331 | 0.4893 |
| | | | | (0.014, 7.586) | |
| 19:19543956:C:T | NA | NA | NA | 1.296 | 0.8243 |
| | | | | (0.131, 12.835) | |
| 19:19543957:C:T | NA | NA | NA | 0.297 | 0.4318 |
| | | | | (0.014, 6.12) | |
| 19:19543971:CG:C | NA | NA | NA | 0.809 | 0.7171 |
| | | | | (0.256, 2.55) | |
| 19:19543986:G:A | 0.046 | 0.838789 | 0.000176104 | 0.801 | 0.6117 |
| | (−0.397, 0.49) | | | (0.34, 1.886) | |
| 19:19543989:G:T | −0.645 | 0.12208 | 5.18E−05 | 0.87 | 0.7024 |
| | (−1.463, 0.173) | | | (0.426, 1.776) | |
| 19:19543998:G:A | NA | NA | NA | 0.322 | 0.3522 |
| | | | | (0.03, 3.502) | |
| 19:19544001:G:A | NA | NA | NA | 1.32 | 0.4261 |
| | | | | (0.666, 2.618) | |
| 19:19544005:C:T | NA | NA | NA | 11.969 | 0.06484 |
| | | | | (0.858, 166.913) | |
| 19:19544011:G:A | NA | NA | NA | 0.274 | 0.08599 |
| | | | | (0.062, 1.201) | |
| 19:19544017:C:G | NA | NA | NA | 0.253 | 0.4132 |
| | | | | (0.009, 6.806) | |
| 19:19544044:T:C | NA | NA | NA | 0.253 | 0.4132 |
| | | | | (0.009, 6.806) | |
| 19:19544049:T:A | NA | NA | NA | 1.174 | 0.6721 |
| | | | | (0.559, 2.464) | |
| 19:19544055:G:A | NA | NA | NA | 2.521 | 0.529 |
| | | | | (0.142, 44.871) | |
| 19:19544077:T:C | NA | NA | NA | 1.772 | 0.6717 |
| | | | | (0.126, 24.975) | |
| 19:19544086:C:T | 0.003 | 0.987072 | 0.000331489 | 0.873 | 0.3101 |
| | (−0.32, 0.326) | | | (0.672, 1.134) | |
| 19:19544119:T:A | −0.09 | 0.811047 | 6.22E−05 | 1.305 | 0.8217 |
| | (−0.837, 0.656) | | | (0.129, 13.177) | |
| 19:19544130:G:A | NA | NA | NA | 5.098 | 0.1729 |
| | | | | (0.49, 53.061) | |
| 19:19544135:G:C | NA | NA | NA | 1.213 | 0.8686 |
| | | | | (0.124, 11.895) | |
| 19:19544152:G:A | NA | NA | NA | 1.439 | 0.6864 |
| | | | | (0.246, 8.418) | |
| 19:19544152:G:C | NA | NA | NA | 33.589 | 0.1009 |
| | | | | (0.504, 2237.75) | |
| 19:19544157:T:G | NA | NA | NA | 7.635 | 0.05077 |
| | | | | (0.993, 58.688) | |
| 19:19544175:G:A | NA | NA | NA | 0.307 | 0.2346 |
| | | | | (0.044, 2.152) | |
| 19:19544184:G:T | NA | NA | NA | 0.337 | 0.4903 |
| | | | | (0.015, 7.398) | |
| 19:19544190:C:T | NA | NA | NA | 1.814 | 0.5286 |
| | | | | (0.285, 11.564) | |
| 19:19544203:C:T | NA | NA | NA | 3.661 | 0.4036 |
| | | | | (0.174, 76.983) | |
| 19:19544206:T:C | −0.089 | 0.785889 | 8.29E−05 | 1.21 | 0.6561 |
| | (−0.736, 0.557) | | | (0.523, 2.797) | |
| 19:19544227:T:C | NA | NA | NA | 0.914 | 0.9324 |
| | | | | (0.115, 7.26) | |
| 19:19544232:C:T | NA | NA | NA | 0.827 | 0.8582 |
| | | | | (0.103, 6.636) | |
| 19:19544244:A:G | NA | NA | NA | 4.059 | 0.02099 |
| | | | | (1.235, 13.339) | |
| 19:19544251:C:T | NA | NA | NA | 0.313 | 0.4142 |
| | | | | (0.019, 5.097) | |
| 19:19544281:C:T | NA | NA | NA | 0.849 | 0.8736 |
| | | | | (0.113, 6.376) | |
| 19:19544287:T:G | NA | NA | NA | 0.328 | 0.5171 |
| | | | | (0.011, 9.576) | |
| 19:19544292:G:C | NA | NA | NA | 0.532 | 0.3498 |
| | | | | (0.142, 1.996) | |
| 19:19544299:T:G | NA | NA | NA | 0.323 | 0.4291 |
| | | | | (0.02, 5.322) | |
| 19:19544326:C:A | NA | NA | NA | 0.535 | 0.6379 |
| | | | | (0.039, 7.245) | |
| 19:19544328:G:A | 0.092 | 0.703812 | 0.000155392 | 1.606 | 0.01947 |
| | (−0.38, 0.564) | | | (1.079, 2.39) | |
| 19:19544328:G:C | NA | NA | NA | 0.861 | 0.8889 |
| | | | | (0.105, 7.036) | |
| 19:19544350:C:T | NA | NA | NA | 2.482 | 0.3788 |
| | | | | (0.328, 18.787) | |
| 19:19544387:C:A | NA | NA | NA | 3.345 | 0.271 |
| | | | | (0.39, 28.72) | |
| 19:19544400:G:A | NA | NA | NA | 0.806 | 0.7602 |
| | | | | (0.202, 3.222) | |
| 19:19544421:C:T | NA | NA | NA | 0.79 | 0.7701 |
| | | | | (0.163, 3.829) | |
| 19:19544436:G:T | NA | NA | NA | 2.616 | 0.3507 |
| | | | | (0.347, 19.701) | |
| 19:19544442:G:A | NA | NA | NA | 6.549 | 0.1266 |
| | | | | (0.588, 72.985) | |
| 19:19544496:G:A | NA | NA | NA | 0.562 | 0.4737 |
| | | | | (0.116, 2.716) | |
| 19:19544517:G:T | NA | NA | NA | 0.325 | 0.4507 |
| | | | | (0.017, 6.04) | |
| 19:19544533:T:G | 0.853 | 0.041129 | 5.18E−05 | 0.303 | 0.2631 |
| | (0.034, 1.671) | | | (0.037, 2.454) | |
| 19:19544548:G:C | NA | NA | NA | 0.395 | 0.3571 |
| | | | | (0.055, 2.853) | |
| 19:19544559:A:G | −0.045 | 0.749179 | 0.000424721 | 1.37 | 0.01328 |
| | (−0.326, 0.235) | | | (1.068, 1.757) | |
| 19:19544571:G:A | NA | NA | NA | 0.895 | 0.8532 |
| | | | | (0.276, 2.902) | |
| 19:19544577:G:A | 0.166 | 0.394832 | 0.000238258 | 1.113 | 0.7074 |
| | (−0.215, 0.548) | | | (0.636, 1.95) | |
| 19:19544578:C:A | 0.166 | 0.394832 | 0.000238258 | 1.113 | 0.7074 |
| | (−0.215, 0.548) | | | (0.636, 1.95) | |
| 19:19544610:G:A | −0.207 | 0.459157 | 0.000113949 | 1.022 | 0.9463 |
| | (−0.759, 0.343) | | | (0.546, 1.913) | |
| 19:19544625:G:T | NA | NA | NA | 0.331 | 0.3931 |
| | | | | (0.026, 4.194) | |
| 19:19544637:C:T | NA | NA | NA | 1.069 | 0.9525 |
| | | | | (0.118, 9.649) | |
| 19:19544653:C:T | 0.237 | 0.533463 | 6.22E−05 | 0.322 | 0.4028 |
| | (−0.509, 0.985) | | | (0.023, 4.575) | |
| 19:19544658:C:T | NA | NA | NA | 0.694 | 0.6958 |
| | | | | (0.111, 4.342) | |
| 19:19544680:G:T | NA | NA | NA | 0.963 | 0.9468 |
| | | | | (0.314, 2.947) | |
| 19:19544748:C:T | NA | NA | NA | 4.038 | 0.0576 |
| | | | | (0.956, 17.057) | |
| 19:19544763:A:T | NA | NA | NA | 0.822 | 0.8489 |
| | | | | (0.11, 6.158) | |
| 19:19544775:T:C | NA | NA | NA | 0.879 | 0.8665 |
| | | | | (0.196, 3.949) | |
| 19:19544778:G:C | NA | NA | NA | 0.44 | 0.3373 |
| | | | | (0.082, 2.355) | |
| 19:19544808:G:C | NA | NA | NA | 3.531 | 0.1202 |
| | | | | (0.719, 17.336) | |
| 19:19544823:G:A | NA | NA | NA | 0.752 | 0.7695 |
| | | | | (0.111, 5.077) | |
| 19:19544827:C:T | NA | NA | NA | 2.219 | 0.3289 |
| | | | | (0.448, 10.994) | |
| 19:19544848:C:G | −0.022 | 0.772778 | 0.00139853 | 0.932 | 0.2781 |
| | (−0.18, 0.135) | | | (0.821, 1.058) | |
| 19:19544853:G:C | −0.099 | 0.792345 | 6.22E−05 | 0.619 | 0.2997 |
| | (−0.847, 0.647) | | | (0.25, 1.533) | |
| 19:19544866:A:AC | NA | NA | NA | 2.791 | 0.4896 |
| | | | | (0.152, 51.287) | |
| 19:19544868:C:T | NA | NA | NA | 0.964 | 0.9731 |
| | | | | (0.112, 8.298) | |
| 19:19544895:G:A | NA | NA | NA | 4.821 | 0.1958 |
| | | | | (0.445, 52.259) | |
| 19:19544902:C:T | NA | NA | NA | 1.276 | 0.8365 |
| | | | | (0.126, 12.874) | |
| 19:19544905:G:C | NA | NA | NA | 2.463 | 0.531 |
| | | | | (0.147, 41.359) | |
| 19:19544911:A:G | 0.132 | 0.549618 | 0.000186463 | 1.238 | 0.4315 |
| | (−0.299, 0.563) | | | (0.727, 2.109) | |
| 19:19544925:C:T | NA | NA | NA | 0.753 | 0.5018 |
| | | | | (0.329, 1.724) | |
| 19:19544928:G:A | NA | NA | NA | 0.613 | 0.5958 |
| | | | | (0.1, 3.746) | |
| 19:19544943:G:A | NA | NA | NA | 0.674 | 0.6533 |
| | | | | (0.121, 3.768) | |
| 19:19544945:C:A | NA | NA | NA | 1.502 | 0.4665 |
| | | | | (0.503, 4.489) | |
| 19:19544954:C:G | NA | NA | NA | 4.883 | 0.04648 |
| | | | | (1.025, 23.257) | |
| 19:19544961:A:G | NA | NA | NA | 1.334 | 0.6299 |
| | | | | (0.413, 4.315) | |
| 19:19545000:G:A | NA | NA | NA | 3.41 | 0.2615 |
| | | | | (0.401, 29.005) | |
| 19:19545000:G:C | NA | NA | NA | 0.542 | 0.4344 |
| | | | | (0.116, 2.52) | |
| 19:19545006:G:A | NA | NA | NA | 1.056 | 0.945 |
| | | | | (0.223, 5.011) | |
| 19:19545039:G:T | −0.051 | 0.891182 | 6.22E−05 | 2.818 | 0.02628 |
| | (−0.798, 0.695) | | | (1.13, 7.027) | |
| 19:19545055:C:T | NA | NA | NA | 0.472 | 0.4106 |
| | | | | (0.079, 2.823) | |
| 19:19545060:C:A | 0.12 | 0.699917 | 9.32E−05 | 2.846 | 0.1682 |
| | (−0.489, 0.73) | | | (0.643, 12.601) | |
| 19:19545072:G:T | NA | NA | NA | 2.596 | 0.371 |
| | | | | (0.321, 21.002) | |
| 19:19545079:G:A | NA | NA | NA | 0.557 | 0.441 |
| | | | | (0.125, 2.472) | |
| 19:19545081:C:G | NA | NA | NA | 0.684 | 0.6064 |
| | | | | (0.161, 2.906) | |
| 19:19545112:G:C | NA | NA | NA | 12.972 | 0.001248 |
| | | | | (2.736, 61.498) | |
| 19:19545124:G:A | NA | NA | NA | 0.339 | 0.5242 |
| | | | | (0.012, 9.47) | |
| 19:19545133:T:A | 0.431 | 0.0839898 | 0.000145027 | 0.799 | 0.5053 |
| | (−0.057, 0.921) | | | (0.412, 1.548) | |
| 19:19545135:G:C | −0.433 | 0.188552 | 8.29E−05 | 1.464 | 0.5906 |
| | (−1.081 , 0.213) | | | (0.365, 5.878) | |
| 19:19545160:C:T | 0.001 | 0.996836 | 0.000549029 | 0.951 | 0.7267 |
| | (−0.25, 0.252) | | | (0.716, 1.263) | |
| 19:19545165:G:A | 0.581 | 0.0782112 | 8.29E−05 | 2.359 | 0.05402 |
| | (−0.065, 1.229) | | | (0.985, 5.647) | |
| 19:19545184:C:G | NA | NA | NA | 0.345 | 0.5037 |
| | | | | (0.015, 7.812) | |
| 19:19545188:G:C | NA | NA | NA | 0.273 | 0.261 |
| | | | | (0.028, 2.629) | |
| 19:19545190:G:A | NA | NA | NA | 0.396 | 0.1717 |
| | | | | (0.105, 1.495) | |
| 19:19545238:G:A | NA | NA | NA | 1.31 | 0.8225 |
| | | | | (0.124, 13.813) | |
| 19:19545238:G:T | NA | NA | NA | 1.007 | 0.9887 |
| | | | | (0.375, 2.702) | |
| 19:19545258:G:A | NA | NA | NA | 0.328 | 0.5049 |
| | | | | (0.012, 8.697) | |
| 19:19545266:C:A | NA | NA | NA | 0.27 | 0.08822 |
| | | | | (0.06, 1.216) | |
| 19:19545274:A:T | NA | NA | NA | 0.302 | 0.3565 |
| | | | | (0.024, 3.85) | |
| 19:19545307:C:A | NA | NA | NA | 0.311 | 0.2175 |
| | | | | (0.049, 1.99) | |
| 19:19545340:A:G | NA | NA | NA | 5.112 | 0.1648 |
| | | | | (0.511, 51.099) | |
| 19:19545355:G:C | NA | NA | NA | 0.57 | 0.4048 |
| | | | | (0.152, 2.138) | |
| 19:19545402:G:A | NA | NA | NA | 0.936 | 0.9444 |
| | | | | (0.147, 5.98) | |
| 19:19545417:G:T | −0.013 | 0.962662 | 0.00010359 | 0.963 | 0.9283 |
| | (−0.592, 0.565) | | | (0.427, 2.174) | |
| 19:19545427:G:T | NA | NA | NA | 0.33 | 0.496 |
| | | | | (0.014, 8.036) | |
| 19:19545436:G:A | NA | NA | NA | 7.475 | 0.004538 |
| | | | | (1.864, 29.98) | |
| 19:19545442:G:A | NA | NA | NA | 4.433 | 0.196 |
| | | | | (0.464, 42.355) | |
| 19:19545493:G:A | NA | NA | NA | 0.056 | 0.3685 |
| | | | | (0, 30.108) | |
| 19:19545505:C:G | 0.086 | 0.722533 | 0.000155386 | 1.07 | 0.8425 |
| | (−0.386, 0.558) | | | (0.548, 2.092) | |
| 19:19545538:T:C | NA | NA | NA | 0.342 | 0.5951 |
| | | | | (0.007, 17.854) | |
| 19:19545546:G:A | NA | NA | NA | 0.313 | 0.3069 |
| | | | | (0.034, 2.902) | |
| 19:19545550:A:G | NA | NA | NA | 0.312 | 0.238 |
| | | | | (0.045, 2.16) | |
| 19:19545573:G:A | NA | NA | NA | 1.339 | 0.814 |
| | | | | (0.118, 15.216) | |
| 19:19545590:G:C | NA | NA | NA | 0.052 | 0.198 |
| | | | | (0.001, 4.663) | |
| 19:19545601:G:A | NA | NA | NA | 0.901 | 0.8737 |
| | | | | (0.248, 3.271) | |
| 19:19545603:C:T | NA | NA | NA | 0.317 | 0.5452 |
| | | | | (0.008, 13.112) | |
| 19:19545604:G:T | −0.029 | 0.896184 | 0.000165745 | 0.9 | 0.7115 |
| | (−0.487, 0.427) | | | (0.515, 1.573) | |
| 19:19545606:G:A | NA | NA | NA | 0.483 | 0.348 |
| | | | | (0.106, 2.207) | |
| 19:19545619:G:T | NA | NA | NA | 1.162 | 0.8289 |
| | | | | (0.297, 4.55) | |
| 19:19545652:C:A | NA | NA | NA | 0.741 | 0.7563 |
| | | | | (0.112, 4.913) | |
| 19:19545652:C:T | NA | NA | NA | 1.788 | 0.663 |
| | | | | (0.131, 24.415) | |
| 19:19545663:G:A | NA | NA | NA | 0.196 | 0.1104 |
| | | | | (0.026, 1.45) | |
| 19:19545664:C:T | 0.736 | 0.077953 | 5.18E−05 | 0.41 | 0.09547 |
| | (−0.081, 1.555) | | | (0.144, 1.17) | |
| 19:19545693:GC:G | NA | NA | NA | 0.789 | 0.76 |
| | | | | (0.173, 3.607) | |
| 19:19545726:G:A | NA | NA | NA | 3.313 | 0.01756 |
| | | | | (1.233, 8.904) | |
| 19:19545747:A:G | 0.069 | 0.502596 | 0.00087016 | 0.856 | 0.06198 |
| | (−0.131, 0.269) | | | (0.728, 1.008) | |
| 19:19545780:C:T | NA | NA | NA | 0.879 | 0.8998 |
| | | | | (0.117, 6.587) | |
| 19:19545804:G:A | NA | NA | NA | 5.243 | 0.02212 |
| | | | | (1.269, 21.67) | |
| 19:19545820:T:C | NA | NA | NA | 0.261 | 0.1609 |
| | | | | (0.04, 1.706) | |
| 19:19545829:A:T | NA | NA | NA | 0.182 | 0.1301 |
| | | | | (0.02, 1.653) | |
| 19:19545834:G:A | NA | NA | NA | 0.32 | 0.4886 |
| | | | | (0.013, 8.028) | |
| 19:19545859:G:A | NA | NA | NA | 0.309 | 0.4096 |
| | | | | (0.019, 5.027) | |
| 19:19545864:C:G | NA | NA | NA | 0.312 | 0.3898 |
| | | | | (0.022, 4.433) | |
| 19:19545876:C:T | NA | NA | NA | 2.126 | 0.5876 |
| | | | | (0.139, 32.483) | |
| 19:19545895:G:A | NA | NA | NA | 4.649 | 0.04081 |
| | | | | (1.066, 20.272) | |
| 19:19545906:T:A | −0.097 | 0.821831 | 5.99E−05 | 0.721 | 0.7187 |
| | (−0.954, 0.758) | | | (0.122, 4.265) | |
| 19:19545933:C:T | NA | NA | NA | 0.534 | 0.2039 |
| | | | | (0.203, 1.405) | |
| 19:19545939:G:A | 0.139 | 0.622137 | 0.000113949 | 1.109 | 0.7531 |
| | (−0.412, 0.691) | | | (0.581, 2.117) | |
| 19:19545952:C:T | 0.013 | 0.96083 | 0.000124309 | 1.66 | 0.168 |
| | (−0.514, 0.542) | | | (0.808, 3.413) | |
| 19:19545969:C:T | NA | NA | NA | 1.024 | 0.9428 |
| | | | | (0.531, 1.978) | |
| 19:19545970:G:A | NA | NA | NA | 0.915 | 0.8706 |
| | | | | (0.314, 2.667) | |
| 19:19545987:C:G | NA | NA | NA | 0.657 | 0.4511 |
| | | | | (0.221, 1.958) | |
| 19:19545987:C:T | NA | NA | NA | 0.974 | 0.9717 |
| | | | | (0.233, 4.07) | |
| 19:19545994:G:A | NA | NA | NA | 1.121 | 0.8951 |
| | | | | (0.205, 6.125) | |
| 19:19545996:C:G | NA | NA | NA | 0.277 | 0.3834 |
| | | | | (0.015, 4.975) | |
| 19:19545996:C:T | NA | NA | NA | 0.294 | 0.2109 |
| | | | | (0.043, 2.002) | |
| 19:19545997:G:A | 0.478 | 0.252123 | 5.18E−05 | 1.2 | 0.7063 |
| | (−0.339, 1.297) | | | (0.464, 3.105) | |
| 19:19546000:G:T | NA | NA | NA | 1.607 | 0.7114 |
| | | | | (0.13, 19.841) | |
| 19:19546009:G:A | NA | NA | NA | 5.836 | 0.04694 |
| | | | | (1.024, 33.26) | |

### Example 3: Association of Lower Risk of Knee Replacement and CILP2

### Phenotype Definitions:

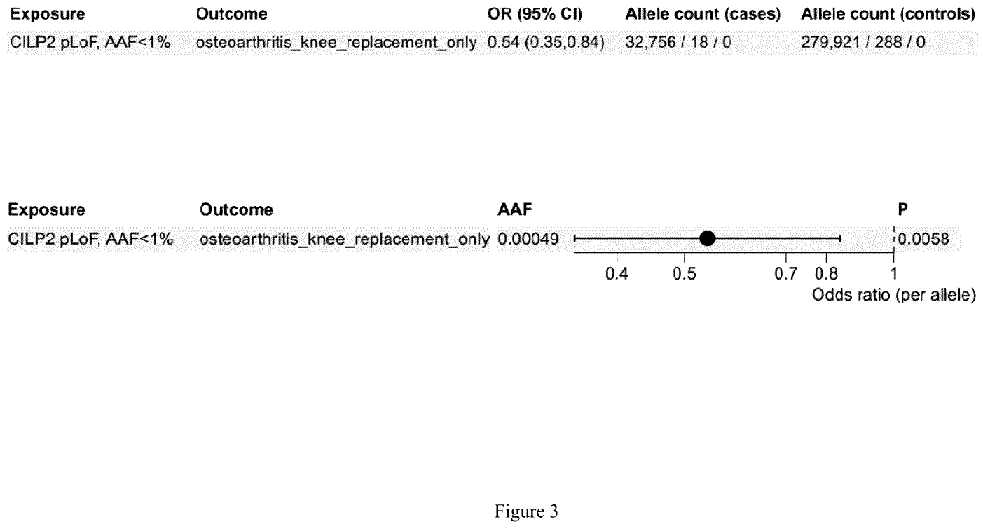

Knee replacement cases were adjudicated in each cohort on the basis of 1) electronic health record history of knee replacement surgery, defined using OPCS-4, CPT-4, ICD-10-PCS, or NOMESCO procedure codes, or 2) self-reported history of knee joint replacement surgery. Individuals not meeting any of the criteria for knee replacement were used as controls. In addition, individuals were excluded from the control group if they had an electronic health record or self-reported history of other forms of arthritis or joint-related symptoms, or a surgical procedure involving a joint. Rare pLoF variants in CILP2 were observed to be associated with a lower risk of knee replacement (odds ratio=0.54; p-value=0.0058) (see, FIG. 3).

5 Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having osteoarthritis or at risk of developing osteoarthritis, the method comprising administering a Cartilage Intermediate Layer Protein 2 (CILP2) inhibitor to the subject.

2. The method of claim 1, wherein the CILP2 inhibitor comprises an inhibitory nucleic acid molecule that hybridizes to a CILP2 nucleic acid molecule.

3. The method of claim 2, wherein the inhibitory nucleic acid molecule comprises an antisense nucleic acid molecule, a small interfering RNA (siRNA), and/or a short hairpin RNA (shRNA).

4. The method of claim 3, wherein the inhibitory nucleic acid molecule comprises an siRNA.

5. The method of claim 3, wherein the inhibitory nucleic acid molecule comprises an antisense nucleic acid molecule.

6. The method of claim 1, wherein the subject is also administered an osteoarthritis therapeutic agent or osteoarthritis therapy.

7. The method of claim 1, further comprising detecting the presence or absence of a CILP2 variant nucleic acid molecule in a biological sample from the subject.

8. The method of claim 7, further comprising administering an osteoarthritis therapeutic agent in an amount that is the same as or less than a standard dosage amount or osteoarthritis therapy to the subject when the CILP2 variant nucleic acid molecule is absent from the biological sample.

9. The method of claim 7, further comprising administering an osteoarthritis therapeutic agent in an amount that is the same as or less than a standard dosage amount or osteoarthritis therapy to the subject when the subject is heterozygous for the CILP2 variant nucleic acid molecule.

10. The method of claim 7, wherein the CILP2 variant nucleic acid molecule comprises a splice-site variant, a stop-gain variant, a start-loss variant, a stop-loss variant, a frameshift variant, a missense variant, an in-frame indel variant, and/or a variant that encodes a truncated CILP2 variant polypeptide.

11. The method of claim 7, wherein the CILP2 variant nucleic acid molecule comprises any one or more of the genetic variations in the genomic nucleic acid molecule set forth in Table 1, or an mRNA molecule produced therefrom, or a cDNA molecule produced from the mRNA molecule.

12. A method of treating a subject having osteoarthritis or at risk of developing osteoarthritis by administering an osteoarthritis therapeutic agent or osteoarthritis therapy, the method comprising:
determining or having determined whether the subject has a Cartilage Intermediate Layer Protein 2 (CILP2) variant nucleic acid molecule, by:
obtaining or having obtained a biological sample from the subject; and
performing or having performed a sequence analysis on the biological sample to determine if the subject has a genotype comprising a CILP2 variant nucleic acid molecule; and
administering or continuing to administer the osteoarthritis therapeutic agent in an amount that is the same as or less than a standard dosage amount or osteoarthritis therapy, and/or a CILP2 inhibitor to a subject that is CILP2 reference;
administering or continuing to administer the osteoarthritis therapeutic agent in an amount that is the same as or less than a standard dosage amount or osteoarthritis therapy, and/or a CILP2 inhibitor to a subject that is heterozygous for the CILP2 variant nucleic acid molecule; or
administering or continuing to administer the osteoarthritis therapeutic agent in a standard dosage amount or osteoarthritis therapy to a subject that is homozygous for the CILP2 variant nucleic acid molecule;
wherein the presence of the CILP2 variant nucleic acid molecule indicates the subject has a decreased risk of developing osteoarthritis.

13. The method of claim 12, wherein the CILP2 inhibitor comprises an inhibitory nucleic acid molecule that hybridizes to a CILP2 nucleic acid molecule.

14. The method of claim 13, wherein the inhibitory nucleic acid molecule comprises an antisense nucleic acid molecule, a small interfering RNA (siRNA), and/or a short hairpin RNA (shRNA).

15. The method of claim 14, wherein the inhibitory nucleic acid molecule comprises an siRNA.

16. (canceled)

17. The method of claim 12, wherein the method comprises administering or continuing to administer the osteoarthritis therapeutic agent in an amount that is the same as or less than a standard dosage amount or osteoarthritis therapy and the CILP2 inhibitor to a subject that is heterozygous for the CILP2 variant nucleic acid molecule.

18. The method of claim 12, wherein the method comprises administering or continuing to administer the osteoarthritis therapeutic agent in an amount that is the same as or less than a standard dosage amount or osteoarthritis therapy and the CILP2 inhibitor to a subject that is CILP2 reference.

19. The method of claim 12, wherein the CILP2 variant nucleic acid molecule comprises a splice-site variant, a stop-gain variant, a start-loss variant, a stop-loss variant, a frameshift variant, a missense variant, an in-frame indel variant, and/or a variant that encodes a truncated CILP2 variant polypeptide.

20. The method of claim 12, wherein the CILP2 variant nucleic acid molecule comprises any one or more of the genetic variations in the genomic nucleic acid molecule set forth in Table 1, or an mRNA molecule produced therefrom, or a cDNA molecule produced from the mRNA molecule.

21. A method of identifying a subject having an increased risk of developing osteoarthritis, the method comprising:
determining or having determined the presence or absence of a Cartilage Intermediate Layer Protein 2 (CILP2) variant nucleic acid molecule in a biological sample obtained from the subject;
wherein:
when the subject is CILP2 reference, then the subject has an increased risk of developing osteoarthritis; and
when the subject is heterozygous or homozygous for the CILP2 variant nucleic acid molecule, then the subject has a decreased risk of developing osteoarthritis.

22-41. (canceled)
